(19) United States
(12) Patent Application Publication
(10) Pub. No.: US 2008/0306019 A1
(43) Pub. Date: Dec. 11, 2008

(54) Dopamine Receptor Agonists in the Treatment and Prevention of Hiv-Induced Dementia

(76) Inventors: Rosemarie M. Booze, Columbia, SC (US); Charles Mactutus, Columbia, SC (US)

Correspondence Address: Ballard Spahr Andrews & Ingersoll, LLP, SUITE 1000, 999 PEACHTREE STREET, ATLANTA, GA, 30309-3915, US

(21) Appl. No.: 11/913,519

(22) PCT Filed: May 3, 2006
(86) PCT No.: PCT/US06/16965

§ 371 Date: May 27, 2008

Related U.S. Application Data
(60) Provisional application No. 60/677,541, filed on May 3, 2005.

(51) Int. Cl. A61K 31/5025; A61K 31/4353; A61K 31/4035; A61K 31/428; A61K 31/165; A61K 31/12; A61K 31/55; A61K 31/551; A61K 31/439; A61K 31/56; A61K 31/52; A61K 31/7072; A61K 31/7076; A61K 31/7064; A61K 31/675; A61K 31/536; A61K 31/496; A61P 25/28; A61P 31/18

(52) U.S. Cl. 514/49; 514/250; 514/284; 514/418; 514/367; 514/619; 514/676; 514/217.02; 514/295; 514/285; 514/171; 514/263.4; 514/50; 514/45; 514/81; 514/230.5; 514/220; 514/253.09

(57) ABSTRACT

Provided herein is a method of protecting a neuron from dysfunction induced by an HIV neurotoxin, comprising contacting the cell with a therapeutically effective dose of a dopamine D1 receptor agonist. Also provided is a method of treating or preventing HIV-1 associated dementia (HAD) in a subject in need of such treatment or prevention, comprising administering to the subject a therapeutically effective dose of a dopamine D1 receptor agonist and estrogenic compound.

### CROSS-REFERENCE TO RELATED APPLICATIONS

This application claims benefit of U.S. Provisional Application No. 60/677,541, filed May 3, 2005, which is hereby incorporated herein by reference in its entirety.

### STATEMENT REGARDING FEDERALLY SPONSORED RESEARCH

This invention was made with government support under Grant DA13137 awarded by NIH/NIDA. The government has certain rights in the invention

### BACKGROUND OF THE INVENTION

A particularly devastating complication of HIV infection is a pervasive form of damage to the brain, HIV-Associated Dementia (HAD) (Buchanan R, et al. JAIDS 2001, 26: 246-255). The overall incidence of severe HAD is estimated at about 30% of the HIV infected population (Dougherty R H, et al. AIDS Read 2002, 12: 69-74). However, HAD occurs more often in HIV-positive IV drug users than HIV-positive non-drug users (Chiesi A, et al. J Acquir Immune Defic Syndr Hum Retrovirol 1996, 11: 39-44; Davies J, et al. AIDS 1997, 11: 1145-1150; Bell J E, et al. Brain 1998, 121: 2043-2052). Neuroimaging and autopsy studies demonstrate that the basal ganglia and frontal lobes are preferentially affected by HAD (Bell J E, et al. Brain 1998, 121: 2043-2052; Berger J R, et al. Neurology 2000, 54: 921-926). These structures may degenerate with chronic psychostimulant (methamphetamine, cocaine) abuse (Holman B, et al. Nucl Med 1992, 33: 1312-1315; Nath A, et al. J Neurovirol 2001, 7: 66-71), eventually leading to a Parkinson type syndrome (Mirsattari et al. Movement Disorders 1998, 13: 684-689). Injection of abused drugs, such as cocaine, has been noted to accelerate the progression of HIV infection to AIDS status and to HAD (Nath A, et al. J Neurovirol 2001, 7: 66-71; Webber M, et al. AIDS 1999, 13: 257-262; Bouwman F H, et al. Neurology 1998, 50: 1814-1820; Margolin A, et al. AIDS Patient Care and STDs 2002, 16: 255-267).

The prevalence of HIV encephalitis is rising despite suppressive antiretroviral therapy (Neuenburg et al., 2002). The unique feature of neurodegenerative pathology associated with HIV is that neuronal cell loss occurs in the absence of neuronal infection. The major cell type harboring productive HIV-1 infection in the nervous system is the perivascular macrophage/microglia. The HIV-1 infection of brain astrocytes is restricted to the expression of regulatory gene products (Epstein, 1998; Nath, 2002). As a result of numerous studies (Nath and Geiger, 1998; Nath, 2002), the concept evolved that the neurotoxicity of viral proteins released into the extracellular environment (as a result of cytopathic infection, restricted infection, active release from infected cells, formation of defective viral particles or shedding of viral coat), plays a key role in neuropathology associated with HIV. It was demonstrated that HIV-1 neurotoxins may cause neuronal damage through common pathways involving the induction of oxidative stress and excitotoxicity (Dewhurst et al., 1996). And despite technical problems associated with the detection of HIV-1 proteins in the CNS, evidence for their presence in the brain tissue of patients with HAD has been obtained (Hofman et al., 1994; Saito et al, 1994; Adamson et al, 1996; Jones et al, 2000; Hudson et al, 2000). Tat mRNA levels are also elevated in brain tissue of patients with HIV dementia (Wesselingh et al., 1993; Wiley et al., 1996). A unique feature of this protein is that it does not get incorporated into the structure of the virus but is actively released in two forms (Tat1-72 formed by the first exon only and Tat1-86-101 formed by the first and second exons) by persistently and productively infected cells without rupture of the cell membrane (Chang et al., 1997). Consistent with this observation, Tat can also be detected in the serum of patients with HIV infection in concentrations of about 1 ng/ml (Westendorp et al., 1995) and in the extracellular matrix in the perivascular compartments in the brain. Along with the other viral proteins, Tat has been detected in the brains of patients with HIV-1 associated brain pathology (Valle et al, 2000) and in the brains of HIV-1-infected primates (Hudson et al, 2000).

Biomarkers of oxidative stress have consistently been detected in brain tissues and cerebrospinal fluid of patients with HIV-associated dementia (Boven. et al., 1999; Haughey et al., 2004). The role for HIV-1 proteins in the development of oxidative stress associated with HIV-1 infection was proposed (Perl and Banki, 2000). It is still debated whether the oxidative stress in HIV is attributable to direct interactions of HIV-1 proteins with neural cells or whether it results from chronic inflammatory reaction induced by the exposure of the CNS tissue to virotoxins. However, it is evident that neurotoxic HIV-1 proteins released from cells harboring HIV-1 may directly trigger oxidative stress, both in cell culture (Kruman et al., 1998; Haughey and Mattson, 2002) and in animal models (Aksenov et al., 2001; Aksenov et al., 2003). Even a transient exposure to HIV-1 proteins may be sufficient to trigger a cascade of events that leads to neuronal degeneration (Nath, 2002). Thus, Tat is an important mediator of neurotoxicity in the HIV-infected brain and investigation of its role in HIV-associated neurodegeneration is important for understanding of the pathogenesis of HIV cognitive and motor dysfunction.

Thus far prevention of or treatment for HAD has not been available. The present invention provides methods for prevention and treatment of HAD.

### BRIEF SUMMARY OF THE INVENTION

In accordance with the purpose of this invention, as embodied and broadly described herein, this invention relates to compositions and methods for protecting a neuron from dysfunction induced by an HIV neurotoxin.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows increased protein carbonyl levels in the striatum of rats microinjected with Tat. Tissue extracts from isolated and immediately frozen striata of control (saline-injected, n=3) and experimental (Tat-injected, 50 μg, 0.021 nmol/g of animal body weight, n=6) animals were prepared as described in (Aksenov et al. 2001a). Immunoblotting of protein carbonyls was carried out using an Oxyblot Kit (Intergen, Purchase, N.Y.). Semiquatitative analysis of protein carbonyl levels was performed Aksevov et al., 2000; 2001). Western blots and Coomassie Blue stained gels were digitized and quantified by computer-assisted imaging. Results presented as mean % vs. non-treated control+SEM. *P<0.05.

FIG. 2 shows neuronal degeneration in the rat striatum 24 hours after single microinjection of Tat. Tat-induced changes in numbers of Fluoro-Jade B positive cells at different post-injection time points are shown in panel A. All data presented as mean %+SEM of saline-injected controls. ** P<0.005 (n=3-5); *P<0.05 (n=3-5); the average number of Jade B-positive cells was less than 1 per field in either Tat-injected or control groups of animals. Fluoro-Jade B positive cells were counted in the sections with injection tracks in the striatum (1 section per animal). Sections were examined using 10× objective of a Nikon fluorescent microscope. Typical images of Fluoro-Jade B stained 24 hr-time point sections are shown in panels B, C, and D. The image of 24 hr-time point brain section obtained from Tat 1-72 injected animal (B) shows Fluoro-Jade B positive cells in the area near the site of injection. Cells positively stained with the fluorescent marker of degeneration rarely occurred near the injection site in the striatum of animals injected with Tat Δ31-61 (C) or saline (D, arrow). The last panel shows the high magnification image of degenerating neurons in the striatum of animals injected with Tat 1-72 (the fragment of 24 hr-time point brain section with the injection track). Arrows point at cell bodies of degenerating neurons and processes labeled with Fluoro-Jade B.

FIG. 3 shows that protein carbonyl levels were increased in the striatum of rats injected with Tat 1-72 at 2 hr and 7 days time points post-injection. (A) The 24 hr time point shows that protein carbonyl immunoreactivity in the striatum of Tat 1-72 injected animals was not significantly different from animals injected with saline and/or Tat Δ31-61. No increase in protein carbonyl levels was detected in the striatum of animals injected with T [symbol] t Δ31-61 at any time point examined All data presented as mean %±SEM of saline-injected controls. *P<0.05 (n=3-5). Signs of reactive astrocytosis were found in the striatum of Tat 1-72 injected animals as late as 7 days following the single microinjection. Increased GFAP immunoreactivity and morpho-logical changes of astrocytes coincided with a second phase of increased protein carbonyls but not with neuronal degeneration.

FIG. 4 shows astroglial cell response following Tat microinjection into the rat striatum peaks at 7 days. Time course of GFAP expression was determined by immunoblotting analysis of striatal extracts and is shown in panel A. Significant increase of GFAP levels was detected 7 days post-injection in the striatum of rats injected with Tat 1-72. Injection of Tat Δ31-61 did not cause significant changes in GFAP production. At 7 days-time point in the striatum of Tat 1-72-injected animals astrocytes exhibited stronger GFAP immunoreactivity, were increased in size and increased numbers of arborizations as compared to controls injected with Tat Δ31-61 or saline. All data presented as mean %±SEM of saline-injected controls. *P<0.05 (n=3-5).

FIG. 5 shows that Tat significantly increased intracellular ROS production. FIG. 6A shows DCF fluorescence in cultures following 2 hours and 48 hours of exposure to different concentrations of Tat 1-72 or T [symbol] t Δ31-61. Neuronal cultures were incubated with 10 μM H₂DCFDA (Molecular Probes, Eugene, Oreg.) for 60 min at 37° C. in PBS. Formation of the oxidation product 2′,7′-dichloro-fluorescein (DCF) was measured using a Bio-Tek Synergy HT microplate reader. Results presented as mean % vs. non-treated control±SEM, n=16/treatment. FIG. 6B shows that Tat increases intracellular ROS production in neurons. Laser confocal/differential interference contrast (DIC) images of intracellular ROS production in cell cultures exposed to Tat 1-72 or T [symbol] t Δ31-61. Images were taken following 2 hours incubation with 50 mM Tat 1-72 or T [symbol] t Δ31-61.

FIG. 6 shows Tat-mediated changes of neuronal viability. Neuronal survival was determined using a Live/Dead viability/cytotoxicity kit from Molecular Probes (Eugene, Oreg.). In accordance with the manufacturer's protocol, neurons were exposed to cell-permeant calcein AM (2 μM) that is hydrolyzed by intracellular esterases, and to ethidium homodimer-1 (4 μM), which binds to nucleic acids. Fluorescence was measured using a Bio-Tek Synergy HT microplate reader (Bio-Tek Instruments, Inc., Winooski, Vt.). Changes in Calcein/Ethidium bromide fluorescence ratio (Live/Dead ratio) following 2 hours and 48 hours of exposure to different concentrations of Tat 1-72 or T [symbol] t Δ31-61 presented as mean % vs. non-treated control+SEM, n of sister cultures analyzed 16/treatment.

FIG. 7 shows ROS production in living cells and not in dead cells after Tat treatment. Living neurons (Orange Calcein-positive) were double-labeled with DCF (Green). TO-PRO-positive dead neurons were not labeled with DCF. Cells were double-labeled with H₂DCFDA/Red-Orange Calcein AM (A) or with H₂DCFDA/TO-PRO-3 iodide (B). Images were obtained using a Nikon C-1 Laser Confocal Microscopy system which supports three channel confocal fluorescence detection while simultaneously capturing scanned differential interference contrast (DIC) images (Nikon Instruments Inc. Melville, N.Y.).

FIG. 8 shows decreased cell viability and increased protein carbonyl immunoreactivity in rat hippocampal neurons at 48 hours after exposure to 50 nM Tat 1-72. Neuronal viability was determined using Calcein/Ethidium bromide (Live/Dead) fluorescence ratio (ns=16). For protein carbonyl immunostaining cell cultures were fixed in acetic alcohol and stained with SYPRO protein stain (Molecular Probes, Eugene, Oreg.) in order to take into account possible variations in total cell protein content from well to well. Readings of SYPRO fluorescence were taken with Synergy HT microplate reader. Cell cultures were treated according to the procedure originally described by (Aksenova et al., 1999). Following normalization of each individual reading to that reflecting total protein content in each well, and subtraction of the average (n=8) normalized 405 nm absorbance value of non-derivatized control to correct for non-specific staining, protein carbonyl immunoreactivity was detected using soluble AP substrate and quantified by measuring 405 nm absorbance (Synergy HT microplate reader). *-marks significant (P<0.05) difference between treated (Tat, 50 nM) and non-treated (control) cell cultures.

FIG. 9 shows that cultures treated with both cocaine (1.6 μM) and Tat (50 nM) exhibited increased intracellular ROS production relative to cultures treated with 50 nM Tat alone after 2 hours of incubation. FIG. 10A shows DCF fluorescence in cultures following 2 hours and 48 hours of exposure to 50 nM Tat 1-72 or 50 nM Tat 1-72+1.6 μM cocaine. Results presented as mean % vs. non-treated control+SEM, ns=8-16. *-marks significant (P<0.05) difference between treated (Tat or Tat+cocaine) and non-treated (control) cell cultures. **-marks significant (P<0.05) difference between Tat-treated and Tat+cocaine-treated cell cultures. FIG. 10B shows Calcein/Ethidium bromide fluorescence (Live/Dead) following 48 hours of exposure to 50 nM Tat 1-72 or 50 nM Tat 1-72+1.6 μM cocaine. Results presented as mean % vs. non-treated control+SEM, ns=8-16. *-marks significant (P<0.05) difference between treated (Tat alone or Tat+cocaine) and non-treated (control) cell cultures. **-marks significant (P<0.05) difference between Tat-treated and Tat+cocaine-treated cell cultures when both experimental groups differ significantly from nontreated control group. No decrease in neuronal viability occur-red in cell cultures subjected to Tat alone or Tat+cocaine after 2 hours of treatment. Cocaine alone did not cause changes in DCF fluorescence and cell death.

FIG. 10 shows that Tat+cocaine significantly increased levels of protein oxidation. Protein carbonyl immunoreactivity was detected following 48-hrs treatment using soluble AP substrate and quantified by measuring 405 nm absorbance with Synergy HT microplate reader. Following normalization of each individual reading to total protein content in the particular well and subtraction of the average (n=8) normalized 405 nm absorbance value of non-derivatized control in order to correct for non-specific staining, results were averaged and presented as mean % vs. non-treated control+SEM, n=8-16/treatment. *-marks significant (P<0.05) difference between treated (Tat alone or Tat+cocaine) and non-treated (control) cell cultures. **-marks significant (P<0.05) difference between Tat-treated and Tat+cocaine-treated cell cultures when both experimental groups differ significantly from non-treated control group.

FIG. 11 shows that GBR-12909 mimicked the effects of cocaine in synergizing with Tat. Calcein/Ethidium bromide fluorescence (Live/Dead) following 48 hours of exposure to 50 nM Tat 1-72, 50 nM Tat 1-72+1.6 μM cocaine or 1-5 μM GBR-12909. Results presented as mean % vs. non-treated control+SEM, n=8-16/group. *-indicates significant (P<0.05) difference between treated and non-treated (control) cell cultures. **-marks significant (P<0.05) difference between Tat-GBR treated and Tat+cocaine treated cell cultures when differ significantly from Tat group and controls. Cocaine and GBR 129092 alone did not produce cell death.

FIG. 12 shows that incubation of rat striatal synaptosomes with either 30 pM gp120 or 60 nM tat significantly decreased [³H]dopamine, and E₂ prevented this decrease. Incubation with mutant TatΔ31-61 had no effect on [3H]dopamine uptake. A one-way ANOVA showed a significant effect [F(4,14)=35.4; p<0.0001) of treatment combination. The reductions in [³H] dopamine uptake were reversed when synapto-somal preparations were co-incubated with 100 nM E₂. Data are expressed as mean±SEM of 4 experiments assayed in duplicate.

FIG. 13 shows kinetic analysis of [³H] dopamine uptake in rat striatal synaptosomal preparations in the presence 30 pM gp120, 60 nM tat, 30 pM gp120+60 nM tat or gp120+tat+100 nM E₂. All curves were best fit to a single-site hyperbola with Kₘ values of approximately 90-109 nmol/mg protein/min. No significant differences in Kₘ values were observed. A significant [F(4,19)=480.9, p<0.0001) effect of treatment was observed on Vₘₐₓ values. Post-hoc analysis with Student Newman-Keuls revealed a significant reduction (30.5%-48.5%) in Vₘₐₓ values for gp120 and tat alone as well as the combination of gp120+tat (p<0.001). Co-incubation with 100 nM E₂ prevented this reduction. Data expressed as mean A SEM of 4 experiments assayed in duplicate.

FIG. 14 shows activity (time) was increased by cocaine, but blocked in animals that had previously received Tat. The overall treatment×drug×session interaction was significant [F(1,53)=4.0, p<0.05]. Specific planned comparisons indicated that the COC-SAL (cocain-saline) animals displayed the anticipated cocaine-induced increase in centrally directed activity [COC-SAL:F(1,53)=4.8, p<0.05] and this was particularly striking [F(1,53)=15.4, p<0.001] relative to the contrary within-session habituation displayed by the SAL-SAL group [F(1,13)=18.2, p<0.001]. The behavior displayed by the SAL-TAT group was not statistically different from the SAL-SAL controls, indicating no effect of Tat per se. Most importantly, however, the COC-TAT treated animals did not signifi-cantly differ from the SAL-SAL controls, but did show that Tat significantly disrupted the development of the cocaine induced increased activity [F(1,53)=4.4, p<0.04]. *-marks the significant within-session habituation of the SAL-SAL controls; **-indicates the significantly increased activity of the COC-SAL animals and the significant difference from COC-TAT animals.

FIG. 15 shows that 17β-E2, not 17α-E2, protects human fetal neurons against the synergistically toxic combination of incubation with Tat [40 nM] plus gp120 [32.5 pM] (HIV Proteins) plus Cocaine (Coc)[1.6 μM]. Control conditions of incubation with Locke's Buffer vehicle or the HIV proteins or Coc only produced similar neuronal death. (Left Panel) Significant interaction of the HIV proteins with Coc, as illustrated by the lines diverging from parallel, produced toxic synergism (*p<0.0008). (Right Panel) Significant neuroprotection by 17β-E2 against HIV proteins w/Coc toxic synergism (#p<0.0001).

FIG. 16 shows that PROG [M] is partially neuroprotective against synergistic neurotoxicity of HIV proteins with cocaine (Coc). Control conditions of incubation with Locke's Buffer vehicle or the HIV proteins or Coc only produced similar neuronal death. (Left Panel) Significant interaction of the HIV proteins with Coc, as illustrated by the lines diverging from parallel, produced toxic synergism (*p<0.0044). (Right Panel) PROG provided partial neuroprotection against HIV proteins w/Coc toxic synergism at all concentrations tested (#p<0.0025) however, the neuroprotection was not concentration dependent.

FIG. 17 shows that T [M] provides concentration-dependent neuroprotection against synergistic neurotoxicity of HIV proteins with cocaine (Coc); DHT's [M] neuroprotection is limited. Control conditions of incubation with Locke's Buffer vehicle or the HIV proteins or Coc only produced similar neuronal death. (Top Panel) Significant interaction of the HIV proteins with Coc, as illustrated by the lines diverging from parallel, produced toxic synergism (*p<0.0087) (Middle Panel). T provides significant concentration-dependent neuroprotection against HIV proteins w/Coc toxic synergism (#p<0.0001). (Bottom Panel). DHT mediated partial protection against synergistic neurotoxicity of HIV proteins w/Coc (#p<0.023); however, the neuroprotection was not concentration dependent.

FIG. 18 shows that in ER mediation of T's protection against synergistic neurotoxicity of HIV proteins with cocaine (Coc); Cholesterol's [M] neuroprotection is incomplete. Control conditions of incubation with Locke's Buffer vehicle or the HIV proteins or Coc only produced similar neuronal death. (Top Panel) Significant interaction of the HIV proteins with Coc, as illustrated by the lines diverging from parallel, produces toxic synergism (*p<0.0001. (Middle Panel) The ER specific antagonist, ICI-182,780 (ICI), completely blocked T-mediated neuroprotection. ICI's toxicity and T's neuroprotection did not differ from vehicle control (#p<0.0001). (Bottom Panel) Cholesterol produced significant, concentration-dependent protection against synergistic neurotoxicity of HIV proteins w/Coc (#p<0.0001); however full protection was demonstrated only at 100 nM.

FIG. 19 shows that specific D1 receptor agonist, SCH23390 (Research Biochemicals), attenuates the enhancement of Tat toxicity by cocaine in primary cultures of rat hippocampal neurons. Groups of cultures prepared in 96-well plates (14 days in culture) were subjected to 50 nM Tat 1-72, 50 nM Tat+1.5 μM cocaine, 50 nM Tat 1-72+10 μM SCH, and 50 nM Tat+1.5 μM cocaine+10 μM SCH. After 48 hours of treatment, cell viability was determined using Calcein/Ethidium bromide method (Live/Dead kit, Molecular Probes). All experimental groups of cultures which received Tat or Tat in combination with cocaine, SCH, or cocaine and SCH, exhibit statistically significant decrease of cell viability after 48 hours of treatment compared to non-treated controls (*): Tat (n=13) versus control (n=14), P=0.0000008; Tat+SCH (n=:15) versus control (n—14), P=0.00000005; Tat+cocaine (n=15) versus control (n=14), P<0.00000001; Tat+cocaine+SCH (n—15) versus control (n=14), P=0.00000009.

FIG. 20 shows that in the presence of oxygen, SIN-1 decomposes to a transitional reactive intermediate and then to the product. In the process of this decomposition, oxygen is converted to superoxide with the spontaneous generation of nitric oxide. Nitric oxide can interact with superoxide resulting in the formation of peroxynitrite.

FIG. 21 shows that increasing concentrations of SIN-1 resulted in a significant (F_{4,14}=39.79; p<0.0001) concentration-dependent increase in DCFH fluorescence in SK-N-SH cells. Post-hoc comparison to control values using Dunnett's Multiple Comparison Test revealed a significant (*p<0.05; **p<0.001) increase in fluorescence at the lowest concentration (5 μM) followed by both the 50 μM and 500 μM concentrations. Data are expressed as mean±SEM of 3 experiments performed in triplicate.

FIG. 22 shows that increasing concentrations of E₂ significantly (F_{4,14}=80.07; p<0.001) reduced SIN-1-induced fluorescence. 50 μM SIN-1 increased fluorescence over control values (vehicle) by nearly 5-fold. Post-hoc multiple comparisons using Student Newman-Keuls revealed that 1 nM E₂ was the threshold concentration for attenuating SIN-1-induced fluorescence. As concentrations of E₂ were increased, significant reductions in fluorescence were observed compared to the previous concentration of E₂ (relative fluorescent units at 0.1>1.0>10>100 nM). Fluorescence values at 100 nM E₂ were not different from control (vehicle) values. The data are expressed as mean±SEM of 4 experiments assayed in triplicate.

FIG. 23 shows that increasing concentrations of both tat and gp120 increased DCFH fluorescence in SK-N-SH cells. Tat concentrations from 0 to 250 nM resulted in a significant increase (F_{4,19}=131.1; p<0.0001) in DCFH fluorescence. The apparent threshold was 50 nM tat. A separate series of wells were incubated in the presence of 250 nM mutant tat_{Δ31-61} (mut) with fluorescent values not different from control. Incubation with gp120 elicited similar results to the tat groups (F_{4,19}=21.61; p<0.0001). The apparent threshold for gp120-induced fluorescence was 20 pM. Data are express as mean±SEM of 4 experiments run in duplicate. *p<0.05, **p<0.001 compared to control values.

FIG. 24 shows SK-N-SH cells incubated in the presence of 30 pM gp120+60 nM tat to induce oxidative stress and increase DCFH fluorescence (control values 5,736±834 RFU). Parallel wells were co-incubated with either 100 nM progesterone, 100 nM E₂, or 100 nM E₂+1 μM ICI 182,780). Analysis revealed a significant effect of hormone (F_{2,30}=94.71; p<0.0001), concentration (F_{4,30}=43.24; p<0.0001) and hormone×concentration interaction (F_{8,30=13.48}; p<0.0001). Neither progesterone nor E₂+ICI 182,780 values were different from control values. Post-hoc comparison with Student Newman-Keuls revealed that concentrations of 1, 10 and 100 nM E₂ were each significantly (p<0.001) from control values. Data are expressed as mean±SEM of 3 experiments assayed in duplicate.

FIG. 25 shows that preincubation of rat striatal synaptosomes with either 30 pM gp120 or 60 nM tat significantly (p<0.01) decreased [³H] dopamine uptake by approximately 70% in the absence of E₂. Incubation with mutant tat_{Δ31-61} had no effect on [3H] dopamine uptake compared to control values. One-way analysis of variance demonstrated a significant effect (F_{4,14}=35.41; p<0.0001) of treatment combination. The observed reductions in [3H] dopamine uptake were reversed when synaptosomal preparations were co-incubated with 100 nM E₂. Data are expressed as mean±SEM of 4 experiments assayed in duplicate.

FIG. 26 shows kinetic analysis of [³H] dopamine uptake in rat striatal synaptosomal preparations in the presence 30 pM gp120, 60 nM tat, 30 pM gp120+60 nM tat or tat+gp120 tat+100 nM E₂. All curves were best fit to a single-site hyperbola with Kₘ values of 90-110 nmol/mg protein/min (TABLE 1). A significant (F_{4,19}=480.9; p<0.0001) effect of treatment was observed on Vₘₐₓ values. Student Newman-Keuls analysis demonstrated a significant reduction (30.5%-48.5%) in Vₘₐₓ values for tat and gp120 alone as well as the combination of tat+gp120 (p<0.001, TABLE 1). Co-incubation with 100 nM E₂ attenuated this reduction (p<0.05). Data are expressed as mean±SEM of 4 experiments assayed in duplicate.

FIG. 27 shows data in adaptation trials for animals tested at 30, 60, and 90 days of age (group 1) and the adult group tested only at 90 days of age (group 2). (A) Mean (±SEM) peak response magnitude for saline and Tat-treated animals, with a significant treatment×age effect for group 1, (B) Mean (±SEM) peak acoustic startle response (ASR) latency with a significant overall age effect for group 1.

FIG. 28 shows data in control trials for animals tested at 30, 60, and 90 days of age (group 1) and the adult group tested only at 90 days of age (group 2). (A) Mean (±SEM) peak response magnitude with a significant age effect for group 1. (B) Mean (±SEM) peak ASR latency with a significant overall age effect for group 1.

FIG. 29 shows mean (±SEM) peak ASR magnitude across PPI trials (8-120 msec) for males tested at 30, 60, and 90 days of age (group 1) and the adult group tested only at 90 days of age (group 2), illustrated as a function of treatment and age. Group 1: Most notable is the leftward peak shift in ISI for maximal inhibition of the response for Tat-injected males at 30 and 60 days of age [χ²(1)=4.67, p=0.031 and χ²(1)=2.62, p<0.106, respectively]. Group 2*: No peak shift was noted but different response curves were noted for saline and Tat-treated males, as indicated by trend analyses.

FIG. 30 shows mean (±SEM) peak ASR latency across PPI trials (8-120 msec) for females tested at 30, 60, and 90 days of age (group 1) and the adult group tested only at 90 days of age (group 2), illustrated as a function of treatment and age. Group 1: Most notable is the significant flattening in the inhibition response in Tat-treated females across PPI trials (8-120 msec) at 30 days of age. At 60 and 90 days of age, Tat significantly altered the inhibition response latency curves, as indicated by trend analyses. Group 2: Trend analyses revealed a significant flattening in the inhibition response in Tat-treated females across PPI trials (8-120 msec).

FIG. 31 shows percent PPI of the startle magnitude [100 msec ISI] for animals tested at 30, 60, and 90 days of age (group 1) and the adult group tested only at 90 days of age (group 2). (A) Mean (±SEM) for saline and Tat-treated animals across age, with a significant treatment×sex effect for group 1. (B) Mean (±SEM) percent PPI with a significant overall age effect for group 1.

FIG. 32 shows mean (±SEM) hippocampal cell density for the five hippocampal regions separate for saline- and Tat-treated rats. # p<0.10, * p<0.05.

FIG. 33(A) shows peak ASR habituation magnitude was significantly reduced in Tat-treated males relative to saline rats. FIG. 33(B) shows mean (±SEM) peak ASR latencies on ASR habituation trials in saline and Tat-treated rats. Data are collapsed across all 36 ASR habituation trials. There was no trial by treatment interaction, suggesting equivalent rates of habituation.

FIG. 34 shows peak ASR magnitude on control trials (0 and 4000 msec combined) revealed a tendency to a significant treatment effect.

FIG. 35 shows mean (±SEM) peak ASR magnitude across ISI (0-4000 msec). Although the ANOVA across PPI trials revealed no overall significant treatment effect, a significant trial×treatment interaction was observed. Most notable is the significant rightward shift in ISI for maximal inhibition of the response for Tat-treated animals.

FIG. 36(A) shows mean (±SEM) peak ASR latency across ISI (0-4000 msec). An ANOVA across PPI trials revealed no significant effects. FIG. 36(B) shows peak ASR latency for saline- and Tat-treated rats across PPI trials (08-120 msec), suggested a slowing in response (4 msec, ˜15%) by Tat, although not statistically significant.

FIG. 37 shows Nissel-stained hippocampal tissue sections through saline (A) and Tat-injected animals (B).

FIG. 38 shows mean (±SEM) peak ASR magnitude in adaptation trials as a function of gp120 dose and drug condition. Post hoc multiple comparison tests for gp120 dose. **p<0.01, *p<0.05.

FIG. 39 shows mean (±SEM) percent PPI of the startle magnitude for ISI 100 msec a function of gp120 dose and drug treatment. No effects were noted.

FIG. 40 shows mean (±SEM) peak ASR magnitude in PPI trials across ISIs [08-120 msec] as a function of gp120 dose and drug treatment. A significant gp120×drug interaction was noted. Simple main effects for drug treatment (solid line) and post hoc tests for gp120 dose separate for each drug treatment (dotted line) were conducted and revealed two statistical significant effects. **p<0.01, *p<0.05, #p<0.10.

FIG. 41 shows mean body weight (±S.E.M.) for each group as a function of day.

FIG. 42 shows mean total activity (±S.E.M.) during a 60-min session, as a function of day, time and group. All animals were habituated to the test environment for two sessions prior to baseline measurement. Rats were injected with SAL prior to the baseline measure (A) and were administered microinjections of Tat or vehicle (VEH) 24-h later. Rats received IV injection of COC (3.0 mg/kg/ml) or SAL and were placed into locomotor activity chambers on day 1 (B) and day 14 (C). n=11-15 rats/group.

FIG. 43 shows mean centrally directed behavior (±S.E.M.) as a function of day, time, and group. All animals were habituated to the test environment for two sessions prior to baseline measurement. Rats were injected with SAL prior to the baseline measure (A and B) and were administered microinjections of Tat or VEH 24-h later. Rats received IV injection of COC (3.0 mg/kg/ml) or SAL and were placed into locomotor activity chambers on day 1 (C and D) and day 14 (E and F). The VEH-SAL and Tat-SAL rats are presented in separate graphs from the VEH-COC and Tat-COC groups for clarity. n=11-15 rats/group. * VEH-COC and Tat-COC, p<0.05.

FIG. 44 shows Tat-mediated changes in availability of TH in the substantia nigra. FIG. 44A shows representative Western blot of HIV 1-infected brain tissues compared to seronegative controls using mouse monoclonal anti-TH antibody as the primary antibody and Alkaline phosphatase-conjugated anti-mouse IgG as the secondary antibody. The molecular weight of TH is approximately 50 kD. FIG. 44B shows TH immunoreactivity in HIV1-infected brain tissues compared to controls. This figure represents the slot blot data. Results presented as mean pixel density (arbitrary units) of HIV 1-infected brain tissues versus control±SEM, n of HIV1-infected brain tissues=7, n of controls=8. * denotes that HIV1-infected brain tissues had significantly lower TH pixel density as compared to seronegative controls, p<0.05.

FIG. 45 shows Tat-mediated changes in availability of DAT in the substantia nigra. FIG. 45A shows representative Western blot of HIV1-infected brains compared to seronegative controls using rabbit polyclonal antibody raised against amino acids 541-620 mapping at the C-terminus of the sodium-dependent dopamine transporter DAT of human origin as the primary antibody and alkaline phosphatase-conjugated anti-rabbit IgG as the secondary antibody. The molecular weight of DAT that is fully glycosylated is approximately 80 kD and the bands at approximately 50 kD are DAT that has one less N-linked glycosylation site. FIG. 45B shows DAT immunoreactivity in HIV1-infected brain tissues compared to controls. This figure represents slot blot data. Results presented as mean pixel density (arbitrary units) of HIV1-infected brain tissues versus control±SEM, n of HIV1-infected brain tissues=7, n of controls=8.

FIG. 46 shows Tat-Mediated changes in NSE density in the substantia nigra. FIG. 46A shows representative Western blot of HIV1-infected brain tissues compared to seronegative controls using rabbit polyclonal anti-NSE antibody as the primary antibody and alkaline phosphatase-conjugated anti-rabbit IgG as the secondary antibody. The molecular weight of NSE is approximately 47 kD. FIG. 46B shows NSE immunoreactivity in HIV1-infected brain tissues compared to controls. This figure represents slot blot data. Results presented as mean pixel density (arbitrary units) of HIV1-infected brain tissues versus control±SEM, n of HIV1-infected brain tissues=7, n of controls=8.

### DETAILED DESCRIPTION OF THE INVENTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a method is disclosed and discussed and a number of modifications that can be made to a number of steps in the method are discussed, each and every combination and permutation of the method and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

It must be noted that as used herein and in the appended claims, the singular forms “a”, “an”, and “the” include plural reference unless the context clearly dictates otherwise. Thus, for example, reference to “a polypeptide” includes a plurality of such polypeptides, reference to “the polypeptide” is a reference to one or more polypeptides and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps.

Those skilled in the art will recognize, or be able to ascertain using no more than routine experimentation, many equivalents to the specific embodiments of the method and compositions described herein. Such equivalents are intended to be encompassed by the following claims.

Provided herein is a method of protecting a neuron from dysfunction induced by an HIV neurotoxin, comprising contacting the cell with a dopamine D1 receptor agonist.

Non-limiting examples of neuronal dysfunction include alterations in neurotransmitter uptake, recycling, and release; neuronal architecture; synaptic transmission, communication, and receptor dynamics; biochemical and signal transduction pathways, and neuronal cell death. Thus, in one aspect, an HIV neurotoxin can result in neuronal cell death. As used herein, neuronal cell death includes apoptosis, necrosis, or other non-specific death of neurons that can occur as a result of exposure to neurotoxins associated with HIV.

As used herein, apoptosis refers to programmed cell death that is signaled by the nuclei when age or state of cell health and condition dictates. Apoptosis is an active process requiring metabolic activity by the dying cell, often characterized by cleavage of the DNA into fragments that give a so called laddering pattern on gels. Cells that die by apoptosis do not usually elicit the inflammatory responses that are associated with necrosis. As used herein, necrosis refers to cell death in response to a major insult, resulting in a loss of membrane integrity, swelling and rupture of the cell. During necrosis, the cellular contents are released uncontrolled into the cell's environment which results in damage of surrounding cells and a strong inflammatory response in the corresponding tissue.

Also provided is a method of treating or preventing HIV-1 associated dementia (HAD) in a subject in need of such treatment or prevention, comprising administering to the subject a therapeutically effective dose of a dopamine D1 receptor agonist.

HIV associated dementia (HAD) is comprised of a spectrum of conditions from the mild HIV-1 minor cognitive-motor disorder (MCMD) to severe and debilitating AIDS dementia complex. Symptoms begin with motor slowing and may progress to severe loss of cognitive function, loss of bladder and bowel control, and paraparesis. A classification system has been formulated for HIV associated dementia, wherein subjects are classified as being Stage 0 (Normal), Stage 0.5 (Subclinical or Equivocal), Stage 1 (Mild), Stage 2 (Moderate), Stage 3 (Severe), or Stage 4 (End-Stage). Thus, the subject of the provided method can therefore be classified as Stage O, Stage 0.5, Stage 1, Stage 2, Stage 3, or Stage 4.

It is understood that dementia affects both cognitive and motor function. Thus, the provided method can treat or prevent impaired cognition resulting from HAD. Thus, the provided method can treat or prevent disrupted motor function resulting from HAD. By “cognitive function” or “cognition” is meant the acquisition of memory or the performance of a learned task. By “motor function” is meant the ability to perform normal ambulatory movements.

By “treat” or “treatment” is meant a method of reducing the effects of a disease or condition. Treatment can also refer to a method of reducing the disease or condition itself rather than just the symptoms. The treatment can be any improvement from the untreated condition and can be but is not limited to the complete ablation of the disease, condition, or the symptoms of the disease or condition. For example, a disclosed method for treatment of HAD is considered to be a treatment if there is a 10% reduction in one or more symptoms of the disease in a subject with the disease when compared to native levels in the same subject or control subjects. Thus, the reduction can be a 10, 20, 30, 40, 50, 60, 70, 80, 90, 100%, or any amount of reduction in between as compared to native or control levels. For example, in the case of HAD, to treat HAD in a subject can comprise improving the disease classification. (e.g. from stage 3 to stage 2, from stage 2 to stage 1, from stage 1 to 0.5 or from stage 0.5 to 0).

As used throughout, “preventing” means to preclude, avert, obviate, forestall, stop, or hinder something from happening, especially by advance planning or action. For example, to prevent HAD in a subject is to stop or hinder the subject from advancing in disease classification (e.g. from stage 0 to stage 0.5, from stage 0.5 to stage 1, from stage 1 to stage 2, from stage 2 to stage 3, or from stage 3 to stage 4).

Dopamine (DA) is present in most parts of the central nervous system (CNS) but in particular in the nigrostriatal pathway comprising the neurons of the substantia nigra and projecting to neurons of the neostriatum and the mesocorticolimbic pathway composed of neurons of the ventral tegmental area connecting with those of the limbic cortex and other limbic structures.

The involvement of the dopaminergic nigrostriatal pathway in extrapyramidal dysfunctions was shown by the discovery that degeneration of this pathway occurs in the brains of patients afflicted with Parkinson's disease. The depletion of dopamine resulting from the degeneration of the nigrostriatal pathway led to the development of dopamine-replacement therapies which are successful in alleviating Parkinson's disease. The hypothesis that dopamine is involved in the pathogenesis of psychosis, in particular schizophrenia, rests on the finding that most antipsychotic drugs are dopamine receptor antagonists and that agents which cause excessive release of dopamine mimic schizophrenia-like states. The mesocorticolimbic pathway has been implicated as the principal dopaminergic pathway involved in the etiology of psychoses. These data explain the dilemma associated with dopamine-related drug therapies: The blockade of the dopaminergic system, desired for reducing psychoses, induces extrapyramidal dysfunctions and vice versa.

It was determined in 1979 that dopamine exerts its effects by binding to two receptors, known as the D1 and D2 receptors. These receptors could be differentiated pharmacologically, biologically, physiologically, and by their anatomical distribution. Pharmacologically, the hallmark of the D1 receptor is to bind the benzazepine antagonist SCH 23390, while that of the D2 receptor is to recognize with high affinity the butyrophenones: spiperone and haloperidol. These two receptors exert their biological actions by coupling to and activating different G protein complexes. The D1 receptor interacts with the Gₛ complex to activate adenylyl cyclase, whereas the D2 interacts with Gᵢ to inhibit cAMP production. The anatomical distributions of these two receptors overlap in the CNS, yet their quantitative ratios differ significantly in particular anatomical areas. With respect to mental disorders, it is noteworthy that both D1 and D2 receptors are present in the nigrostriatal and mesocorticolimbic pathways.

For 10 years, this two-subtype classification has accounted for most of the activities attributed to the dopaminergic system. However, this classification was dramatically changed with the application of recombinant DNA technology to the molecular characterization of the dopamine receptors. The application of homology screening techniques not only led to the deciphering of the molecular structures of the D1 and D2 receptors, but also led to the characterization of three new dopamine receptors: D3, D4, and D5.

The cloned dopamine receptors (D1-D5) can be divided into two groups of receptors that correspond to the D1 and D2 receptor classification that had been previously identified pharmacologically. The D1 and D5 receptors have a D1-like pharmacology, whereas the D2, D3, and D4 receptors have a D2-like pharmacological profile. In general, the D1 and D2 receptor mRNAs have a wider distribution and are more abundant in the CNS as compared to their pharmacologically related counterparts. The D5 receptor mRNA, for example, is restricted to specific thalamic and hypothalamic nuclei and to the cells of the hippocampus, whereas the D1 receptor mRNA is detected in numerous regions of the CNS. Similarly, cells expressing D3 receptor mRNA are detected in far fewer nuclei than those expressing D2 receptor mRNA. The wider distributions of cells expressing D1 and D2 receptor mRNA may be reflective of the broader number of functions mediated by these receptors in the CNS, including the modulation of cognitive, sensorimotor, and neuroendocrine effects, as compared to more limited functions that may be mediated by the other dopamine receptor types.

Given the intronic organization of the D2, D3, and D4 genes, multiple mRNA transcripts may be generated by each gene by alternative splicing. While variant and truncated forms of the D3 and D4 receptors have been reported, two forms of the D2 receptor that differ by a 29-amino-acid insertion in the third cytosolic loop have been studied most extensively. In situ hybridization studies in pituitary and brain suggest that both mRNAs are expressed in the same cells, with the longer D2 form (444 amino acids) being the more abundant species. The relative ratios of D2(444) and D2(415), however, do vary with brain area, and some studies have suggested that the D2 receptor forms may be differentially regulated with antipsychotics or denervation.

Dopamine (DA) is a catecholamine neurotransmitter found predominately in the central nervous system. It is synthesized from the amino acid tyrosine, which is converted to L-dihydroxyphenylalanine (L-DOPA) by the enzyme tyrosine hydroxylase (TH). Dihydroxyphenylalanine is converted to dopamine by the enzyme DOPA decarboxylase (or aromatic amino acid decarboxylase) which is found in the cytoplasm. Thus, the herein disclosed methods can comprise contacting the cell or administering to the subject a therapeutic amount of a TH agonist. Examples of TH agonists are known in the art and include TCH-346, COX-2 inhibitors (e.g., Celecoxib, Valdecoxib, Rofecoxib), or gene therapies. For example, TH can be agonized by recombinant expression of TH. Nucleic acid and amino acid sequence for tyrosine hydroxylase are known in the art. As an example, human tyrosine hydroxylase nucleic acid and amino acid sequence can be found in Accession No. NM_{—}000360.

The dopamine transporter (DAT) is a plasma membrane transport protein thought to control extracellular DA concentrations through the recapture into DA nerve terminals of DA that has been released during the process of neurotransmission. DA transport and its accumulation in nerve endings was first characterized in 1969, a number of years after the description of tissue uptake of the related neurotransmitter norepinephrine (NE). During the early 1980's, the development of radiolabeled probes with some selectivity for the DAT advanced understanding of the distribution, pharmacology and regulation of the DAT.

The DAT is a member of a large family of Na⁺/Cl⁻ dependent transporters, including the closely related NE transporter (NET) as well as transporters for serotonin, GABA, glycine, proline, creatine, betaine, and taurine. DAT is a 620 (human) amino acid protein that includes 12 transmembrane domains (TMDs), with both the amino- and carboxy-termini residing within the cytoplasm. Monoamine transporter sequences are least conserved at these termini and a large extracellular loop occurring between TMD 3 and TMD 4 and most conserved in the putative TMDs.

Amino acid sequence for the dopamine receptors are known in the art. As an example, human dopamine D1 receptor amino acid sequence can be found in Accession No. NP_{—}000785. Human dopamine D2 receptor amino acid sequence can be found in Accession No. P60026. Human dopamine D3 receptor amino acid sequence can be found in Accession No. AAA73929. Human dopamine D4 receptor amino acid sequence can be found in Accession No. NP_{—}000788. Human dopamine D5 receptor amino acid sequence can be found in Accession No. NP_{—}000789.

The dopamine D1 receptor agonist of the provided methods can be any non-naturally occurring polypeptide, peptided mimetic or small molecule capable of binding and activating the dopamine D1 receptor. In one aspect, the dopamine D1 receptor agonist is not a naturally occurring neurotransmitter. In another aspect of the provided methods, the dopamine D1 receptor agonist is not dopamine. Dopamine is generally not used to treat HIV-1 associated dementia as it actually exacerbates the disease. This can be explained in part by evidence that dopamine can increase HIV-1 virus replication. Thus, in another aspect of the provided method, the dopamine D1 receptor agonist does not increase HIV-1 replication.

In another aspect of the provided methods, the dopamine D1 receptor agonist does not bind the dopamine D2 receptor. In another aspect of the methods, the dopamine D1 receptor agonist does not bind the dopamine D3 receptor. In another aspect of the methods, the dopamine D1 receptor agonist does not bind the dopamine D4 receptor. In another aspect of the methods, the dopamine D1 receptor agonist does not bind the dopamine D5 receptor.

Non-limiting examples of dopamine D1 receptor agonist include Bromocriptine, Pergolide, Ropinirole, Pramipexole, Entacapone, Tolcapone, Fenoldopam, Apomorphine, Dihexadine, IPX-750, Cabergoline, A68930, SKF38393, CY208-243, SKF81297, NNC01-0012, and SCH23390. It is understood that other known or newly discovered dopamine D1 agonists can be used or adapted for use in the disclosed methods. For example, D1 receptor agonists can be identified using standard screening methods known in the art. As a non-limiting example, a cell based functional assay for high-throughput drug screening for D1 receptor agonists is described by Jiang N, et al. (Acta Pharmacol Sin. 2005 October; 26(10): 1181-6), which is hereby incorporated by reference herein in its entirety for the teaching of said assay and compounds discovered by same. Briefly, cells (e.g., CHO cells) can be transfected with an expression vector comprising D1 receptor cDNA and a reporter gene (e.g., pCRE/TA/Luci) and used to screen compound libraries for activation of the reporter gene. A number of extracts have been identified by this method (e.g., SBG492). Thus, SBG492, and any other compound identified by this or any other such screening method are contemplated for use in the disclosed compositions and methods.

Thus, provided herein is a method of protecting a neuron from dysfunction induced by an HIV neurotoxin, comprising contacting the cell with a therapeutically effective dose of Bromocriptine, Pergolide, Ropinirole, Pramipexole, Entacapone, Tolcapone, Fenoldopam, Apomorphine, Dihexadine, IPX-750, Cabergoline, A68930, SKF38393, CY208-243, SK 81297, NNC01-0012, SCH23390, or a combination thereof.

Also provided is a method of treating or preventing HIV-1 associated dementia (HAD) in a subject in need of such treatment or prevention, comprising administering to the subject a therapeutically effective dose of Bromocriptine, Pergolide, Ropinirole, Pramipexole, Entacapone, Tolcapone, Fenoldopam, Apomorphine, Dihexadine, IPX-750, Cabergoline, A68930, SKF38393, CY208-243, SKF81297, NNC01-0012, SCH23390, or a combination thereof.

Psychostimulants such as cocaine and methamphetamine enhance Tat-induced neurotoxicity. Thus, the dysfunction induced by an HIV neurotoxin or HAD can be a result of HIV infection in the presence of a psychostimulant. The psychostimulant can be cocaine, methamphetamine, amphetamine or methylphenidate. Thus, in another aspect of the provided methods, the dopamine D1 receptor agonist can attenuate Tat-induced neurotoxicity enhanced by a psychostimulant.

The herein provided methods can further comprise administering to the subject a therapeutic amount of an estrogen receptor agonist. Thus, in one aspect the estrogen receptor agonist can comprise any form of estrogen (e.g., 17 beta-estradiol, conjugated equine estrogens, synthetic conjugated estrogens, and esterified estrogens). An example of conjugated equine estrogens is Premarin. An example of synthetic conjugated estrogens is Cenestin. Examples of esterified estrogens are Estratab and Menest. The estrogen receptor agonist can also be administered in combination with Raloxifene or a Progestogen (e.g., medroxyprogesterone acetate). As testosterone is converted to estrogen by neurons, in another aspect of the provided method, the estrogen receptor agonist can comprise testosterone. In another aspect, the estrogen receptor agonist is an estrogen receptor type alpha agonist.

Thus, provided herein is a method of protecting a neuron from dysfunction induced by an HIV neurotoxin, comprising contacting the cell with a dopamine D1 receptor agonist and an estrogen receptor agonist. Also provided is a method of treating or preventing HIV-1 associated dementia (HAD) in a subject in need of such treatment or prevention, comprising administering to the subject a therapeutically effective dose of a dopamine D1 receptor agonist and an estrogen receptor agonist.

As disclosed herein, the beneficial effect of the estrogen receptor agonist is due in part to an ability to stabilize dopamine transporter (DAT). The net result is a decrease in dopamine availability. Thus, the herein provided methods can further comprise administering to the subject a therapeutic amount of a compound that decreases dopamine availability.

In addition to ameliorating the effects of HIV neurotoxins on neuronal cell death and dementia, the herein provided methods can further comprise administering to the subject a therapeutic amount of an antiretroviral compound. Antiretroviral drugs inhibit the reproduction of retroviruses such as HIV. Antiretroviral agents are virustatic agents which block steps in the replication of the virus. The drugs are not curative; however continued use of drugs, particularly in multi-drug regimens, can significantly slow disease progression. There are three main types of antiretroviral drugs, although only two steps in the viral replication process are blocked. Nucleoside analogs, or nucleoside reverse transcriptase inhibitors (NRTIs), act by inhibiting the enzyme reverse transcriptase. Because a retrovirus is composed of RNA, the virus must make a DNA strand in order to replicate itself. Reverse transcriptase is an enzyme that is essential to making the DNA copy. The nucleoside reverse transcriptase inhibitors are incorporated into the DNA strand. This is a faulty DNA molecule that is incapable of reproducing. The non-nucleoside reverse transcriptase inhibitors (NNRTIs) act by binding directly to the reverse transcriptase molecule, inhibiting its activity. Protease inhibitors act on the enzyme protease, which is essential for the virus to break down the proteins in infected cells. Without this essential step, the virus produces immature copies of itself, which are non-infectious. A fourth class of drugs called fusion inhibitors block HIV from fusing with healthy cells.

Thus, the antiretroviral compound can comprise one or more molecules selected from the group consisting of protease inhibitors [PI], fusion inhibitors, nucleoside reverse transcriptase inhibitors [NRTI], and non-nucleoside reverse transcriptase inhibitors [NNRTI].

Thus, the antiretroviral compound of the provided methods can be a PI, such as a PI selected from the group consisting of Indinavir, Amprenavir, Nelfinavir, Saquinavir, Fosamprenavir, Lopinavir, Ritonavir, and Atazanavir, or any combinations thereof.

Thus, the antiretroviral compound of the provided methods can be a fusion inhibitor, such as for example Enfuvirtide.

Thus, the antiretroviral compound of the provided methods can be a NRTI, such as a NRTI selected from the group consisting of Abacavir, Stavudine, Didanosine, Lamivudine, Zidovudine, Zalcitabine, Tenofovir, and Emtricitabine, or any combinations thereof.

Thus, the antiretroviral compound of the provided methods can be a NNRTI, such as a NNRTI selected from the group consisting of Efavirenz, Nevirapine, and Delavirdine.

The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration; the route of administration; the rate of excretion of the specific compound employed; the duration of the treatment; drugs used in combination or coincidential with the specific compound employed and like factors well known in the medical arts. For example, it is well within the skill of the art to start doses of the compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose.

The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. For example, the disclosed anti-retroviral compounds can be administered at published dosages, such as those approved for human use, e.g., in the treatment of HIV-1 infection. A typical daily dosage of the disclosed dopamine D1 receptor agonists used alone can range from about 0.001 mg/kg to up to 50 mg/kg of body weight or more per day, depending on the factors mentioned above.

Bromocriptine can be administered from about 3.75 to 40 mg per day. Pergolide can be administered from about 0.75-5 mg per day. Ropinirole can be administered from about 1.5-24 mg per day. Pramipexole can be administered from about 1.5-4.5 mg per day. Entacapone can be administered from about 600-2000 mg per day. Tolcapone can be administered from about 200-600 mg per day. Fenoldopam can be administered from about 100-500 mg per day. Apomorphine can be administered from about 2-6 mg per day.

A typical initial dose of Bromocriptine is about 1.25 mg 2× per day. A typical initial dose of Pergolide is about 0.05 mg 1× per day. A typical initial dose of Ropinirole is about 0.25 mg 3× per day. A typical initial dose of Pramipexole is about 0.125 mg 3× per day. A typical initial dose of Entacapone is about 200 mg per day. A typical initial dose of Tolcapone is about 100 mg 2× or 3× per day. A typical initial dose of Fenoldopam is about 100 mg per day. A typical initial dose of Apomorphine is about 4 mg per day.

Raloxifene is typically administered at about 60 mg or 120 mg per day. Conjugated equine estrogen is typically administered at about 0.625 mg or 1.25 mg/day. Medroxyprogesterone acetate is typically administered at about 2.5 mg per day. Low-dose, transdermal estrogen is typically administered at about 25 μg/day. 17 β-estradiol is typically administered at about 0.05 mg/day.

The compositions can also be administered in vivo in a pharmaceutically acceptable carrier. By “pharmaceutically acceptable” is meant a material that is not biologically or otherwise undesirable, i.e., the material can be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The pharmaceutical composition can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. Thus, the disclosed compositions can be administered intracranially intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The compositions may be administered orally or parenterally (e.g., intravenously, intramuscular injection, by intraperitoneal injection, transdermally, extracorporeally, intracranially, topically or the like, including topical intranasal administration or administration by inhalant. As used herein, “intracranial administration” means the direct delivery of substances to the brain including, for example, intrathecal, intracisternal, intraventricular or trans-sphenoidal delivery via catheter or needle. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These can be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffier, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### EXAMPLES

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

### Example 1

### Oxidative Stress Associated with Tat Neurotoxicity

Neurotoxic properties of Tat have been demonstrated in cell culture studies (Nath, 1999; Nath et al., 2002), but little information was initially available regarding the effects of Tat in vivo. Lesion formation following stereotaxic microinjections of Tat into the rat striatum has been investigated (Bansal et al., 2000). Microinjection of Tat (1-50 μg) found dose-dependent Tat-mediated damage in the rat striatum. This study provided an in vivo model for characterization of molecular mechanisms of Tat neurotoxicity.

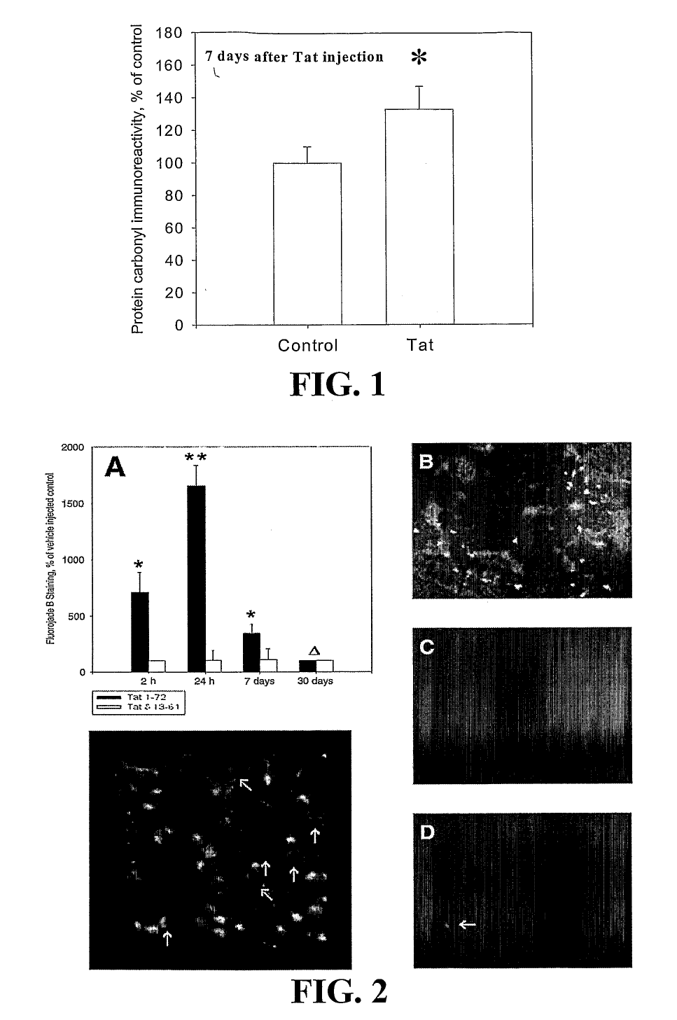

As demonstrated herein, neurotoxicity of Tat 1-72 (first exon) microinjected into the striatum of adult male rats was associated with increased oxidative modification of proteins. Immunoblotting analysis revealed a significant increase in protein carbonyl levels in striatal tissue extracts obtained from rats 7 days after stereotaxic microinjections of Tat (FIG. 1). The dose of Tat injected into the rat striatum was equivalent to the dosage used in the similar studies by other researchers (Jones et al., 1998).

Protein carbonyls are an important biomarker of oxidative stress-associated cell damage. Carbonyl derivatives are formed by reactive oxygen species-mediated oxidation of side chains of some amino acid residues. Carbonyl groups also may be introduced into proteins by glycation and reactions with glycoxidation and lipid peroxidation products (Butterfield and Stadtman, 1997). Results of the immunoblotting analysis were confirmed by anti-protein carbonyl immunohistochemistry. Immunostaining of brain sections demonstrated that an area of prominent protein carbonyl immunoreactivity surrounded an injection site in the striatum of Tat-injected rats.

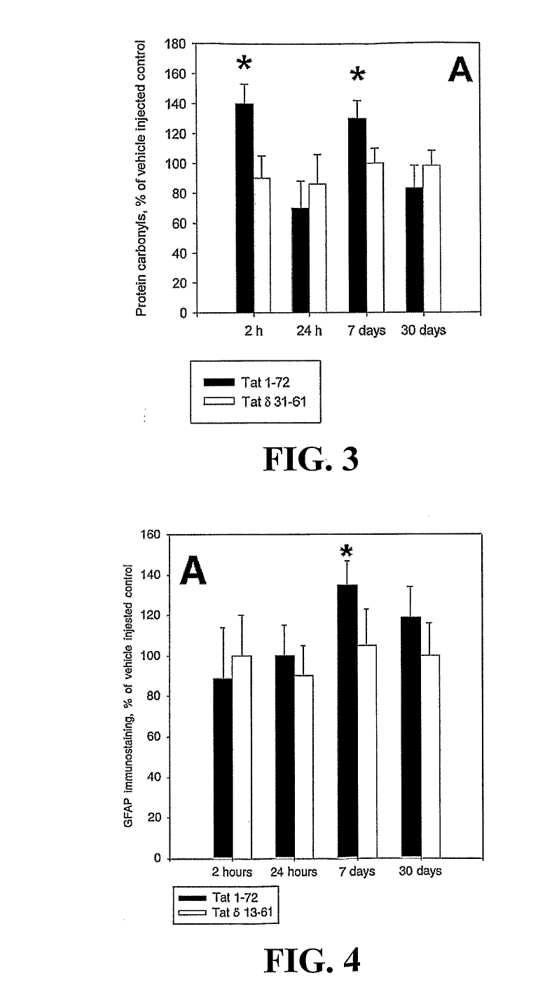

These results indicate that Tat-mediated neurotoxicity is associated with oxidative damage. However, the question remained whether oxidative damage occurred early in the process of Tat neurotoxicity or whether it was secondary to the inflammation and cell death (Jones et al., 1998; Bansal et al., 2000). Thus temporal progression of histopathological changes induced by a single microinjection of Tat 1-72 (50 μg) was examined. The temporal relationships between neuronal degeneration, macrophage/microglial infiltration of the lesion area, reactive astrocytosis, and protein oxidation in the rat striatum was examined following microinjections of Tat 1-72 or the biologically inactive analog Tat Δ31-61. In this study, degenerating neural cells detected by Fluoro-Jade B staining (FIG. 2) and increased protein oxidation determined by protein carbonyl immunoblotting (FIG. 3) were observed in the striatum as soon as 2 hours following the microinjection of Tat.

Results of in vivo studies (Bansal et al., 2000; Aksenov et al, 2001; 2003) indicate that increased oxidative damage of macromolecular cell components is an early event, rather than a marker of late stages of neuronal degeneration, caused by Tat. Thus, oxidative stress induced by protein neurotoxins in HIV-infected brain plays an important role in the pathogenesis of HIV-associated neurodegenerative pathology. As demonstrated herein, degeneration of neurons following a single injection of Tat into the rat brain promotes accumulation of reactive microglia and a delayed astrogliosis. Nevertheless, the late astrogliosis was not sufficient to cause additional neuronal cell death.

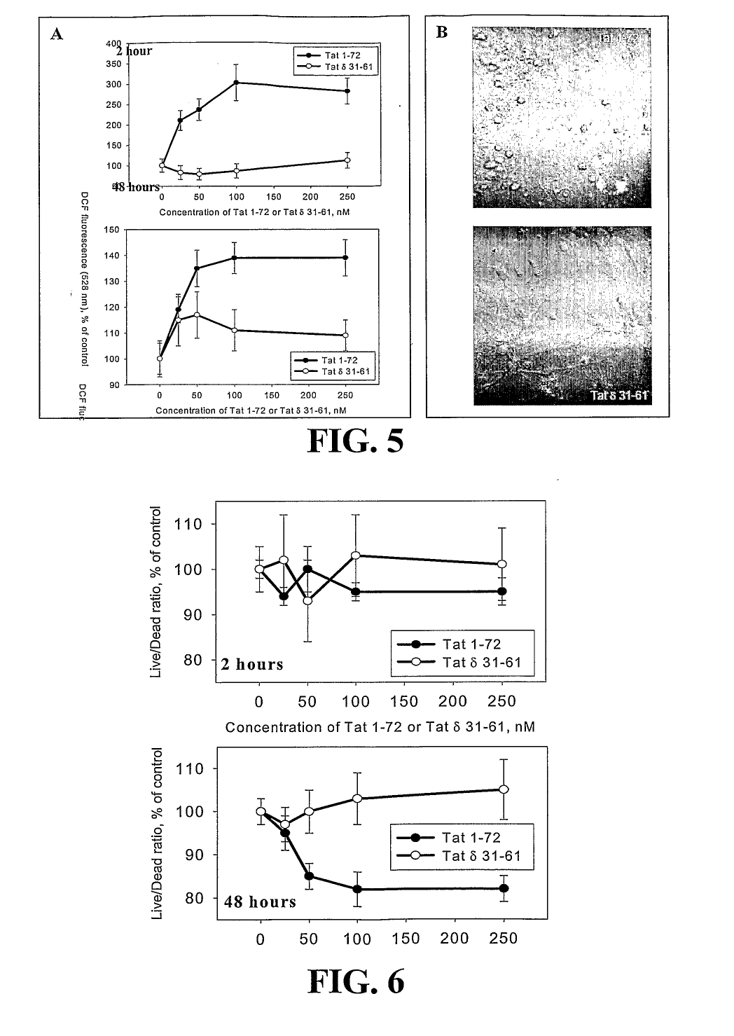

The direct neurotoxic effects of Tat on primary neuronal cell cultures was further examined (see Aksenova et al, 2005 for review of cell culture models) at two time points (2 hours and 48 hours) post-treatment. Using a cell culture model allows detection of early Tat effects that precipitate neuronal cell death. As demonstrated herein, Tat caused significant increases in reactive oxygen species (ROS) production in cultured cells at 2 and 48 hours (Tat 1-72 concentrations ranged from 25 nM up to 250 nM) as determined by increased DCF fluorescence (FIG. 5).

Cell viability, as determined using a differential live/dead fluorescent staining technique, did not decrease after 2 hours incubation; however, after 48 hours 20% of the cells were dead (maximal effect at 50-100 nM Tat) (FIG. 6).

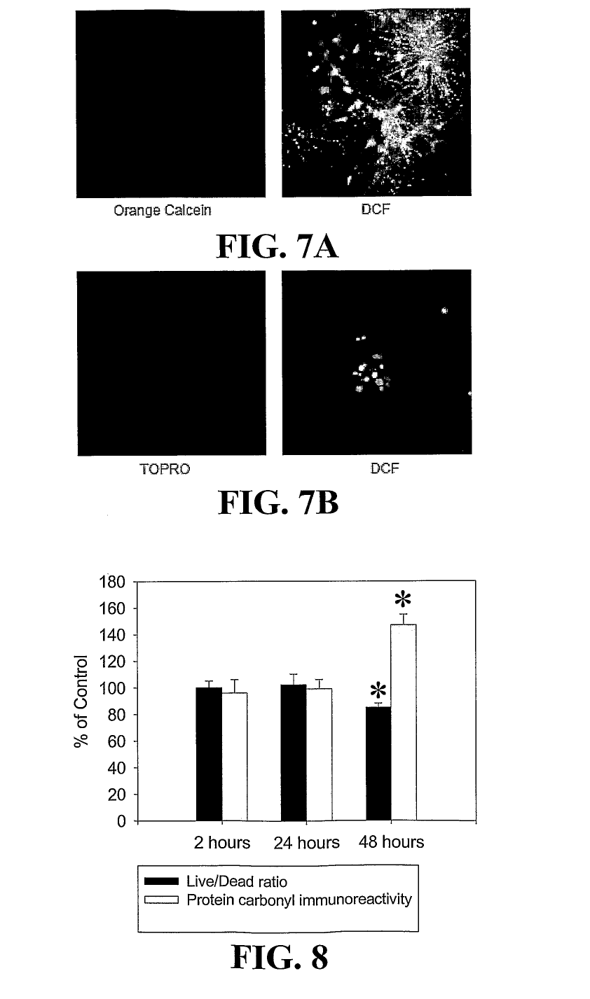

Confocal images of DCF fluorescence in Tat-treated cells indicated that ROS production was associated with living cells (Orange calcein-positive) and not with dead cells (TO-PRO-positive) (FIG. 7).

Increased protein oxidation was not evident at the earliest time point after treatment when signs of increased production of ROS have been detected (see above studies—2 hours). Changes in levels of oxidized proteins coincided with the late decrease of cell viability, 48 hours after the exposure of cell cultures to Tat (FIG. 8).

### Example 2

### Synergistic Effects of Cocaine on Tat Toxicity: Evidence for Enhancement of Tat-Induced Oxidative Stress in Rat Hippocampal Neurons by Cocaine

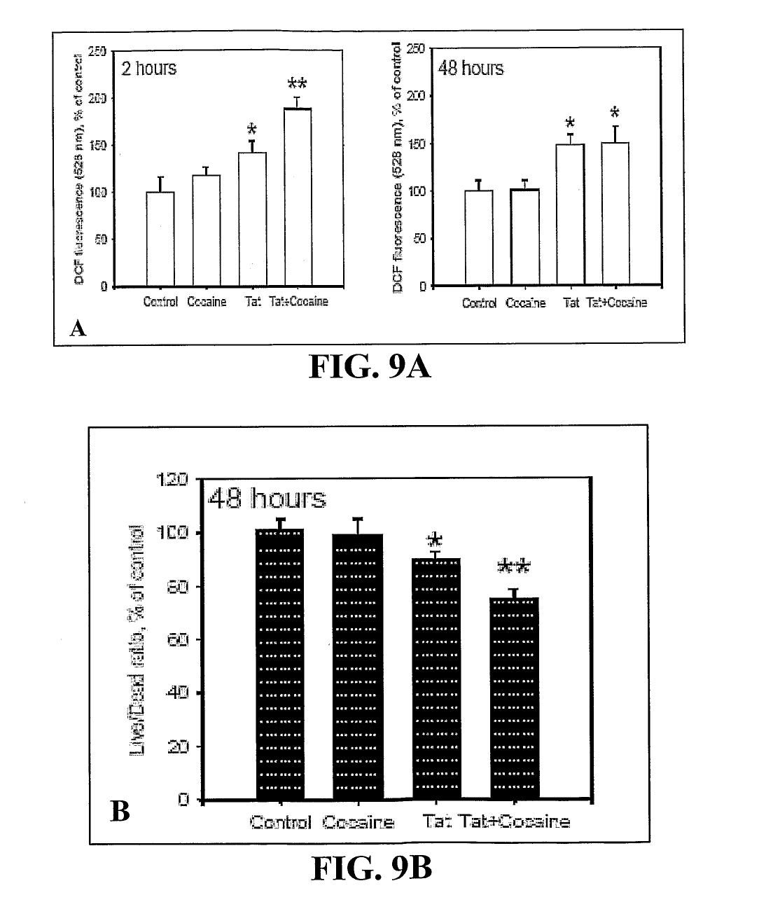

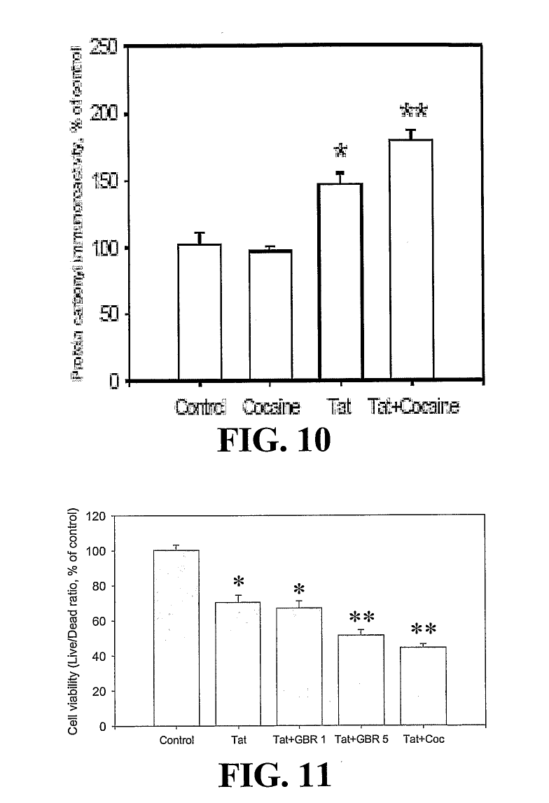

The ability of cocaine to modulate Tat-induced intracellular ROS production and neurodegeneration in cultured rat hippocampal fetal neurons was examined. The dose of cocaine that we used in culture (1.6 μM) was within the physiological levels experienced by human cocaine drug users (Evans et al., 1996). No toxicity of 0.1-10 μM cocaine was observed in cultured rat hippocampal neurons (FIG. 9). After 48 hours of treatment differences in DCF fluorescence between Tat+cocaine and Tat-treated cultures were not significant (FIG. 9); however, neuronal cell death was significantly higher in cultures subjected to both Tat and cocaine (FIG. 9). Increased production of ROS in cell cultures following incubation with Tat and Tat+cocaine is associated with increased protein carbonyl immunoreactivity. Levels of protein oxidation in cell cultures that were treated with both Tat and cocaine were higher than in cultures treated with just Tat alone (FIG. 10). Thus, cocaine enhances Tat-mediated changes in neuronal oxidative status, which in turn leads to increased oxidative cell damage and degeneration.

### Example 3

### Role of Dopamine and Estrogen in Cocaine-Tat Neurotoxicity

Monoamine transporter proteins (DAT, NET and SERT) are primary sites for cocaine actions with respect to reward/drug abuse (Kalivas, 1995). To determine if DAT is a target for cocaine-Tat interactions in producing HIV-dementia, a 50 nM dose of Tat was used to induce moderate toxicity (15-20% cell viability decrease after 48 hours of treatment) in rat hippocampal cell cultures. The selective dopamine transport inhibitor, GBR 12909 (Sigma-Aldrich, St. Louis, Mo.) (1-(2-bis(4-fluorphenyl)-methoxy)-ethyl)-4-(3-phenyl-propyl)piperazine) is a selective inhibitor of DA uptake. GBR-12909 exhibits selective affinity to dopamine transporter of about 500 times higher than cocaine. Doses from 0.05 to 6 μM are not neurotoxic to rat hippocampal neurons in culture.

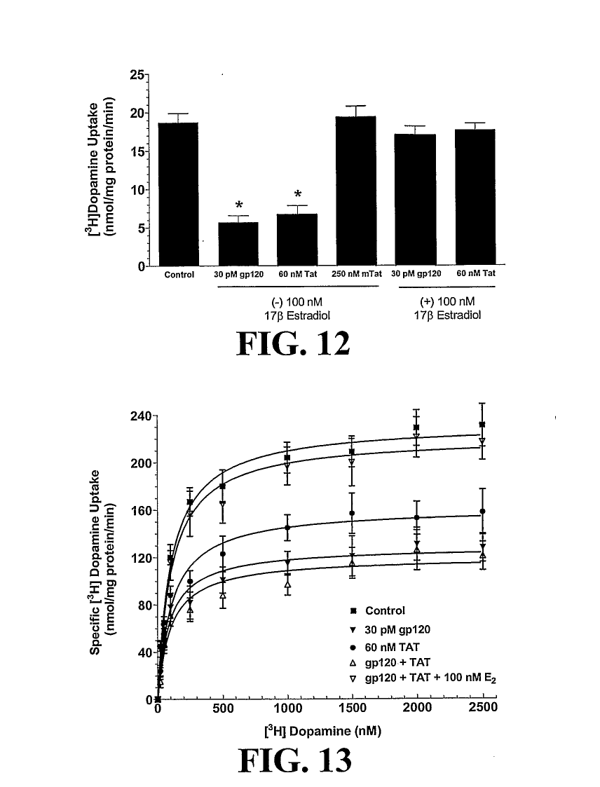

Thus, the data in FIG. 11 indicates that the dopamine transporter protein (DAT) is a site of cocaine action for producing cell death in conjunction with Tat exposure. The selective DAT inhibitor, GBR-12909, synergized with Tat in producing cell death in a manner similar to that of cocaine. Given that cocaine and many other drugs of abuse inhibit DAT in order to produce rewarding effects, inhibition of DA uptake can be critical in Tat-drug abuse neurotoxicity. Neither cocaine nor GBR 12909 kill neurons in isolation, so uptake inhibition by itself is not neurotoxic and other mechanisms must be involved. Therefore, the ability of Tat to disrupt synaptosomal dopamine uptake and the ability of DAT to mediate the neuroprotectant effects of estrogen were examined. The in vitro effects of tat and gp120 on [³H] dopamine uptake was examined in rat striatal synaptosomes. Even short-term exposure to tat and/or gp120 resulted in a significant reduction in dopamine uptake (FIG. 12, 13). Co-incubation with 100 nM E2 essentially prevented this effect and Km and Vₘₐₓ values remained near control values.

### Example 4

### In Vivo Effects of Tat

### Methods

Animals. Fifty-seven adult female Sprague-Dawley rats (70 days old) (Harlan Laboratories, Inc., Dublin, Va.) had an Intracath IV catheter (22 ga, Becton/Dickinson General Medical Corp., Grand Prarie, Tex.) implanted as a subcutaneous (dorsal) port for chronic IV injections as previously described (Mactutus et al., 1994). Ovariectomy was also performed on all rats during the same surgical procedure.

Intra-accumbal TAT injection. A 2 treatment×2 drug, design was used. Thus, rats received bilateral injections of TAT or vehicle into the nucleus accumbens core and were subsequently administered an IV injection of cocaine (3.0 mg/kg/injection) or saline. Rats were administered intra-accumbal TAT or VEH injections according to methods previously described (Bansal et al., 2000). Briefly, 15 μg of Tat were injected bilaterally, using a Hamilton Syringe coated with Sigmacote, at the following coordinates: 1.8 mm anterior, ±1.6 mm lateral, and 7.0 mm ventral from bregma on the skull surface.

Locomotor activity. The activity monitors were 16-cm diameter, round open-field chambers (Flex-Field, San Diego Instruments, San Diego, Calif.) that detected free movement of animals by infrared photocell interruptions. This equipment used an infrared photocell grid (32 emitter/detector pairs) to measure total locomotor activity. The activity monitors were used to specifically measure centrally directed behavior, a measure sensitive to integrity of the nucleus accumbens, by assessing the number and location of photocell interruptions within a test session.

Rats in the saline-vehicle (SAL-VEH; n=14), saline-Tat (SAL-Tat; n=15), cocaine-vehicle (COC-VEH; n=14), and cocaine-Tat (COC-Tat; n=14) groups were habituated to the locomotor activity chambers for two 60-min sessions, one/day. A baseline measure of activity was obtained on day three. All rats received intra-accumbal Tat or SAL injections within 48 hrs after baseline measurement. Twenty-four hours after rats received intra-accumbal injections of Tat or VEH, IV COC or SAL was administered and the animals were immediately placed into locomotor activity chambers.

Drug Treatment. The cocaine treatment was administered as a bolus injection delivered in a volume of 1 ml/kg body weight (15 s), and was followed by flushing (15 s) with 0.2 ml heparinized (2.5%) saline (i.e., the approximate volume of the catheter). The dose of cocaine hydrochloride (3.0 mg/kg/day; Sigma, St. Louis, Mo.) was calculated on the weight of the salt and dissolved in saline for an injection volume of 1 ml/kg. The IV dosing regimen used has been shown to produce a pharmacokinetic profile in rats with a t1/2 of approximately 12-13 minutes in plasma (Booze et al., 1997).

### Results

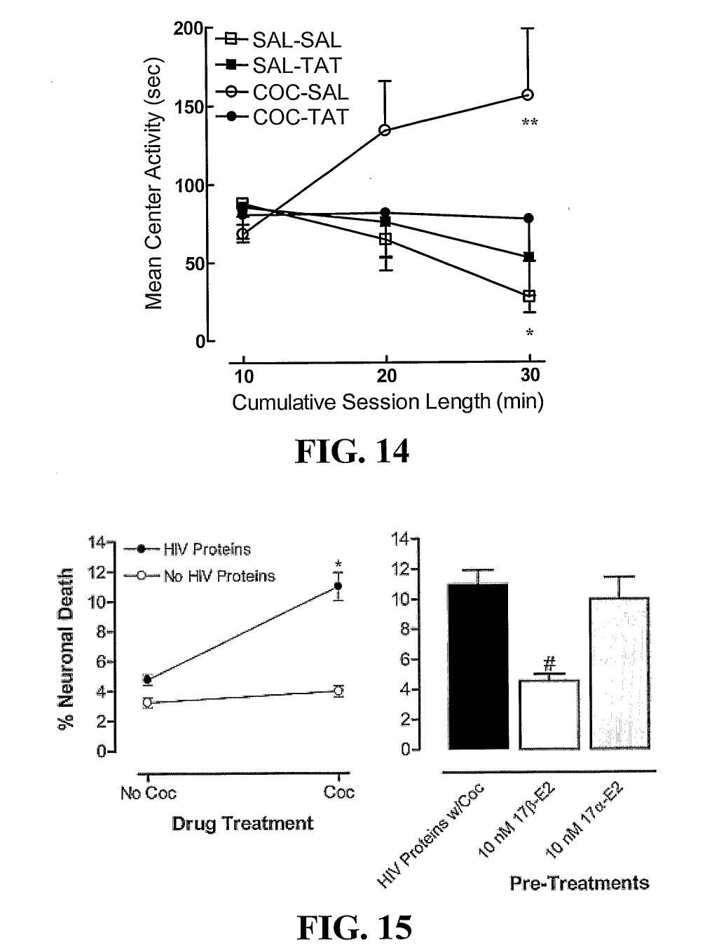

As shown in FIG. 14, animals which had received bilateral Tat injections into nucleus accumbens did not respond to cocaine challenge 24 hours later. Thus, treatment with Tat could have produced DAT dysfunction, and therefore, cocaine treatment 24 hours later could not produce the expected cocaine-induced increase in locomotor activity. This 24 hour post-Tat injection time point is after protein carbonyls/oxidative stress occurs (Aksenov et al., 2003; see above).

### Example 5

### Neurotoxicity Induced by Cocaine and HIV Proteins can be Blocked with Estrogen Receptor Agonists

### Methods

Culture of Human Brain Cells: Neuronal cultures were prepared as described previously (Magnuson D S, et al. Ann Neurol 1995, 37: 373-380; Nath A, et al. J Virol 1996, 70: 1475-1480; New D, et al. J Neurovirol 1997, 3: 168-173). Human fetal brain specimens of gestational age 12-14 weeks were obtained with the consent of women opting for elective termination of pregnancy. Protocols were carried out with strict adherence to the guidelines of the National Institutes of Health (NIH) and with approval from the University of Kentucky Institutional Review Board. Briefly, after mechanical dissociation, the cells were suspended in Opti-MEM with 5% heat-inactivated fetal bovine serum, 0.2% N2 supplement and 1% antibiotic solution (penicillin G sodium 104 units/ml, streptomycin sulfate 10 μg/ml and amphotericin-B 25 μg/ml) (GIBCO). The cells were maintained in culture flasks at 37° C. in a humidified atmosphere of 5% CO₂ and 95% air for at least a month. At least 3 days before the experiment was performed the cells were plated in flat bottom 96 well plates coated with poly-d-lysine. Sample cultures were stained for the neuron-specific enolase, microtubule-associated protein-2, synaptophysin, and glial fibrillary astrocytic protein (GFAP). The cultures were thus characterized as more than 80% homogenous for neurons. The remaining cells were predominantly astrocytes (GFAP positive) with less than 1% microglia/macrophages, as determined by RCA-1 lectin and CD68 staining (New D, et al. J Neurovirol 1997, 3: 168-173; Gelbard H A, et al. J Virol 1994, 68: 4628-4635; New D R, et al. The Journal of Biological Chemistry 1998, 273: 17852-17858). Sample cultures were further characterized for the presence of ER-α and ER-β and dopaminergic neurons. ERs were visualized in 5-10% of cultured astrocytes and neurons in the cytoplasm and nuclei of both by immunostaining. Antigenicity for dopamine and dopamine transporter were co-localized in nearly 60% of neurons. Dopamine receptor D1A was seen in 50% of cells while receptor D2 was seen in 40% of cells. ER co-localized with cells staining for dopamine as well as D1A and D2 receptor containing neurons.

Recombinant Tat and gp120 Proteins: Recombinant Tat was prepared as described previously (New D, et al. J Neurovirol 1997, 3: 168-173; Ma M, et al. J Virol 1997, 71: 2495-2499) with minor modifications. The tat gene encoding the first 72 amino acids was amplified from and inserted into an E coli vector PinPoint Xa-2 (Promega). This construct allowed the expression the Tat1-72 protein as a fusion protein naturally biotinylated at the N-terminus. The biotinylated Tat protein was purified on a column of soft release avidin resin, cleaved from the fusion protein using factor Xa, eluted from the column, then desalted with a PD10 column. The Tat protein was further purified of endotoxin by passage through a polymycin B, cyanogen bromide, immobilized on cross-linked 4% beaded agarose column (Sigma). All purifications steps contained dithiothreitol to prevent oxidation of the proteins. Tat Protein was more than 95% pure as determined by SDS-PAGE followed by silver staining. Analysis by HPLC using a C4 column showed a single symmetrical peak. Western blot analysis showed that this preparation contained both monomeric and dimeric forms of Tat1-72. The functional activity of Tat1-72 was confirmed using a transactivation assay in HL3T1 cells containing an HIV-1 LTR-CAT construct. Chiron Corporation made a gift of gp120 from HIVSF2 which was described previously (Turchan J, et al. BMC Neurosci 2001, 2: 3). Briefly, recombinant gp120 was made in a Chinese Hamster Ovary cell line. Purification yielded 95% pure gp120 as determined by Western blot analysis. The Tat and gp120 preparations contained less than 1 pg/ml endotoxin as determined using a Pyrochrome Chromogenic test kit (Associates of Cape Cod, Inc., Falmouth, Mass.). The Tat proteins were stored in a lyophilized form and gp120 as a stock solution in water at −80° C. in endotoxin-free siliconized microfuge tubes until taken for experimentation. Tat and gp120 are highly susceptible to degradation and loss of biological activity with each freeze-thaw cycle. Therefore, single aliquots were used for each experiment. Any remaining solution was discarded. Prior work has shown that no significant toxicity is seen with Tat less than 80 pM, gp120 less than 40 pM, or Tat (40 nM) plus gp120 (30 pM) (Turchan J, et al. BMC Neurosci 2001, 2: 3). For these studies, we used subtoxic dosage levels of Tat1-72 at 40 nM and gp120 at 32.5 pM.

Drug levels: Cocaine hydrochloride obtained from the NIDA Drug Supply Program was solvated in Locke's buffer (pH 7.2) immediately prior to use. The dosage of cocaine (500 ng/ml) in culture translates to 1.6 pM, which is several orders of magnitude less than studies demonstrating a direct cytotoxic effect in culture (Gu J, et al. Neurotoxicology 1993, 14: 19-22; Nassogne M, et al. Proc Natl Acad Sci USA 1995, 92: 11029-11033; Bennet B, et al. Neurosci Lett 2003, 153: 210-214). Fetal brain levels of cocaine of 1750+/−250 ng/ml were associated with fetal plasma levels of 500 ng/ml following repeated daily dosing in rat (Robinson S, et al. J Pharmacol Exp Ther 1994, 271: 1234-1239). The plasma levels of cocaine from fetuses of mothers exposed to cocaine using the rat IV model Mactutus C, et al. Neurotoxicology and Teratology 1994, 16: 183-191) was 500 ng/ml as this represents 1/5 the peak brain levels in the fetus. This is also a concentration within the physiological levels experienced by human IV cocaine drug users (Evans S, et al. J Pharmacol Exp Ther 1996, 279: 1345-1356). Thus 500 ng/ml (1.6 μM) represents a physiologically relevant dose of cocaine to the neurons. Cocaine levels as high as 16 μM are not neurotoxic in human fetal neuronal cultures (Turchan J, et al. BMC Neurosci 2001, 2: 3). In these studies the cocaine solution was diluted such that addition of 1:10 (v/v) to the culture dishes resulted in a final concentration of 1.6 μM. This dose of cocaine was added concurrently with the Tat and gp120 proteins to the cultures.

Neurotoxicity Assay: At the time of the experimental treatment, the culture media was replaced with Locke's buffer containing (in mM) 154 NaCl, 5.6 KCl, 2.3 CaCl₂, 1 MgCl₂, 3.6 NaHCO₃, 5 glucose, 5 N-2-hydroxyethylpiperazine-N′2-ethanesulfonic acid (HEPES) and 1% antibiotic solution (penicillin G sodium 104 units/ml, streptomycin sulfate 10 μg/ml and amphotericin-B 25 μg/ml) (pH 7.2). Cells were incubated for 15 hours in Locke's buffer or with subtoxic concentrations of the HIV proteins, Tat1-72 (40 nM) plus gp120 (32.5 pM) (Turchan J, et al. BMC Neurosci 2001, 2: 3), or cocaine (1.6 μM), or the HIV proteins plus cocaine to demonstrate the synergistic effect on cell death of HIV proteins with cocaine. To replicate the stereoisomer specific neuroprotective properties of estrogen, the cultures were treated with either 170-E2 (10 nM), or 17α-E2 (10 nM) immediately preceding addition of the HIV proteins plus cocaine. To investigate the neuroprotective properties of other gonadal steroids, the cells were treated with various concentrations of PROG or T, immediately followed by exposure to the HIV proteins plus cocaine. To determine the mechanism of T's neuroprotection, the cultures were treated with various concentrations of DHT (5α-Androstan-17β-ol-3-one; 17β-Hydroxy-5α-androstan-3-one), or ICI-182,780 (100 nM) plus T (10 nM) before the HIV proteins plus cocaine were added. Various concentrations of cholesterol were used as treatment prior to incubation with the HIV proteins plus cocaine to further characterize the nature of gonadal steroid neuroprotection. ICI-182,780, DMSO (10 nM), β-cyclodextrin (100 nM) and ethanol (10 nM) were also tested for neurotoxic/protective effects. ICI-182,780 and 17α-E2 were solvated in DMSO. 170-E2, T, PROG and cholesterol were β-cyclodextrin encapsulated to provide water solubility. Ethanol was used to solvate DHT. Tat1-72 was produced as indicated above, whereas gp120 was a gift from Chiron Corporation. The ICI-182,780 compound was obtained from Tocris Cookson, Inc. (Ellisville, Mo.). All other chemical agents were supplied by Sigma Chemicals (St. Louis, Mo.). At the end of the incubation period neuronal death was assessed by trypan blue (Sigma) exclusion assay as described previously (Vongher J and Frye C. Pharmacol Biochem Behav 1999, 64: 777-785; Magnuson D S, et al. Ann Neurol 1995, 37: 373-380; Nath A, et al. J Virol 1996, 70: 1475-1480). In brief, each experiment was conducted in triplicate and at least three independent squads were treated with each pharmacological agent. Five randomly selected fields from each well were photographed using an inverted microscope (Nikon Diphot, 40×) and coded by a technician blinded to the well treatments. Viable and dead neurons in each photomicrograph were counted before decoding for statistical analysis. In representative fields the neurotoxicity assay was verified by false color visualization using the MCID digital camera-based system interfaced with a computer (Imaging Research, Ontario, Canada).

The neuronal and glial phenotypes seen in these cultures have been well characterized immunohistochemically and morphologically in our hands (Magnuson D S, et al. Ann Neurol 1995, 37: 373-380; Nath A, et al. J Virol 1996, 70: 1475-1480) as well as by others. Furthermore, the glia readily attach to the surface of the culture dish, whereas neurons do not (Fried G, et al. Eur J Neurosci 2004, 20: 2345-2354). Astrocytic attachment to the culture dish is requisite for the survival of the neurons, which in turn, attach to the substrate of glia. In this way, the glia are seen in a specific plane of microscopy that differs from the microscopic plane in which the neurons are seen.

Analysis: Data are expressed as percent neuronal cell death/total viable neurons (means±SEM). A 2 (presence or absence of the viral proteins gp120 and Tat)×2 (presence or absence of cocaine) analysis of variance (ANOVA) design was employed to ascertain the potential for synergistic viral protein/cocaine-induced neurotoxicity. A significant interaction of the HIV proteins with Coc, as graphically illustrated by the lines diverging from parallel, provided evidence of toxic synergism. One-way ANOVAs were used to assess the neuroprotective potential of the various gonadal steroid pre-treatments. Regression analysis was used to determine concentration-effect relationships (Winer B: Statistical Principles in Experimental Design, 2 edn. New York: McGraw Hill; 1971). Planned Tukey-Kramer comparisons were used to determine specific treatment effects. An α level of p<0.05 was considered significant for all statistical tests employed. Computer assisted analyses utilized BMDP Statistical Software, Release 7, Los Angeles, Calif. (1993) and SPSS Statistical Software release 11.5.0, SPSS, Inc., Chicago, Ill. (2004).

### Results

Clear and robust synergistic neurotoxicity of the HIV proteins Tat and gp120 was repeatedly observed when combined with a physiologically relevant dose of cocaine. False color visualization of the intensity of trypan blue within neurons verified the assay for cell death and demonstrated the synergistic toxicity of cocaine with the HIV proteins. Furthermore, this neurotoxicity is precluded by pretreatment with 10 nM dose of 17β-E2, but not with 17α-E2. Neither the HIV proteins, Tat plus gp120, nor cocaine alone were more toxic than the Locke's buffer control; however, in combination they produced synergistic neurotoxicity (FIG. 15). The ANOVA confirmed the presence of a significant interaction of the HIV proteins with cocaine (F(1,24)=15.38, p<0.0008; n=6 each point) (FIG. 15 Left Panel). The presence of this significant neurotoxic effect was demonstrated in every experiment at p<0.005. The stereoisomers of estradiol demonstrated a significant treatment effect against this toxicity (F(2,15)=6.95, p<0.007). The 17 β-stereoisomer of estradiol demonstrated significant neuroprotection (F(1,24)=24.71, p<0.0001; n=at least 3 each point). The percent neuronal death with 17α-E2 treatment was not significantly different from the synergistic toxicity control (FIG. 15 Right Panel).

DMSO at 10 nM (0.1%) served as the solvent for 17α-E2 and for ICI-182,780; therefore, it was tested for toxicity and was not significantly different from Locke's buffer incubation. β-Cyclodextrin (100 nM) served as an encapsulating carrier to enhance solubility for the steroids 17β-E2, T, PROG and cholesterol and was therefore tested for neuroprotection. When coincubated with the toxic combination of HIV proteins plus cocaine, this carrier also had no significant effect on cell death.

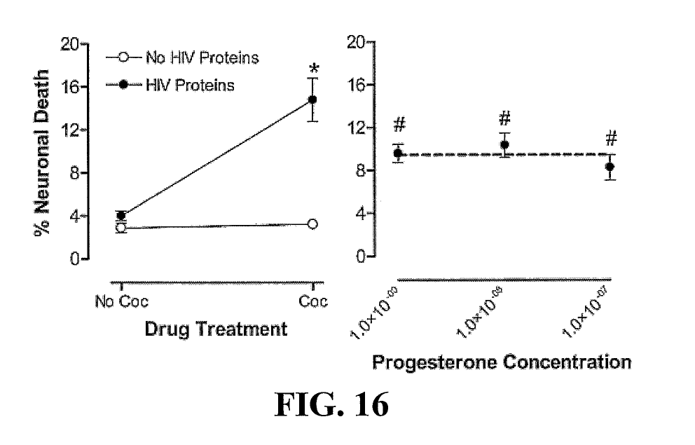

To study the effect of PROG on HIV protein and cocaine synergistic toxicity cultures were treated with 1 nM, 10 nM, and 100 nM doses of PROG prior to adding the toxic combination of HIV proteins and cocaine (FIG. 16 Right Panel). PROG demonstrated significant neuroprotection (F(1,28) 11.03, p<0.0025; n=5 each point). There was no concentration-dependent effect on percent neuronal death (F(1,28)<1.0). Further, the neuroprotection was incomplete at these concentrations as the magnitude of cell death was significantly greater than that observed with Locke's Buffer control (ps<0.01 for all concentrations tested). FIG. 16 Left Panel shows the significant interaction of the HIV proteins with cocaine confirming the presence of synergistic neurotoxicity (F(1,16)=10.97, p<0.0044; n=5 each point;).

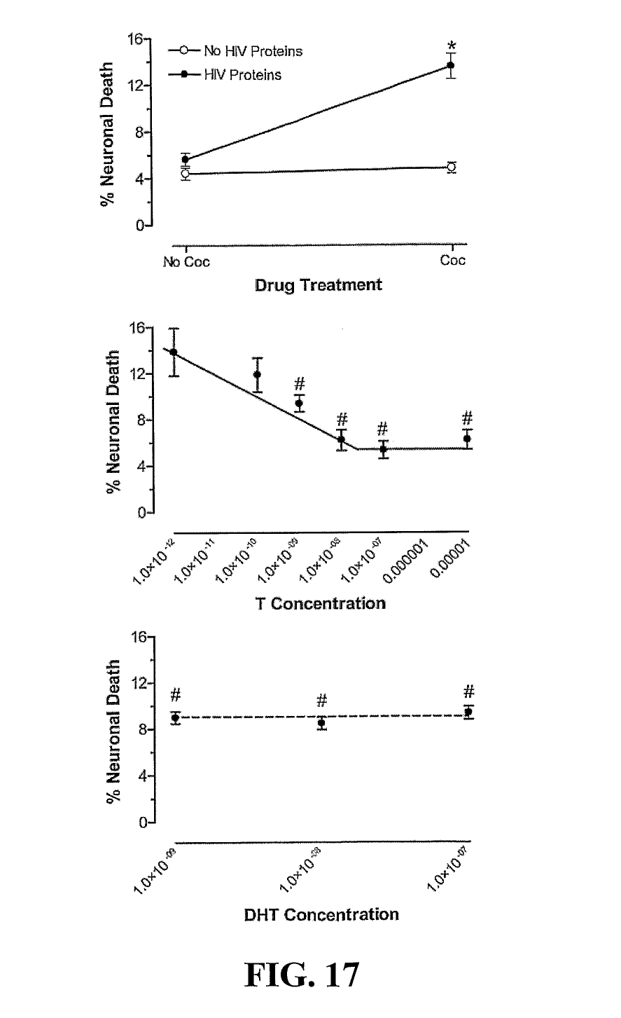

Concentrations of 1 pM, 100 pM, 1 nM, 10 nM, 100 nM and 10 μM of T were tested against the synergistically neurotoxic combination of HIV proteins and cocaine (FIG. 17). A significant interaction of the HIV proteins with cocaine (F(1,20)=8.47, p<0.0087; n=at least 6 each point) confirmed the presence of synergistic neurotoxicity (FIG. 17 Top Panel). T treatment provided significant neuroprotection (F(1,57)=12.71, p<0.0007; n=at least 4 each point). Specifically, there was a concentration-dependent effect of T with a significant linear decrease in percent neuronal death as a function of increasing dose (F(1,57)=22.56, p<0.0001) (FIG. 17 Middle Panel). The neuroprotective effect of T was characterized against the negative control of Locke's buffer vehicle and the positive control of the synergistically toxic combination of HIV proteins with cocaine. A ceiling effect of maximal neuroprotection at T concentrations of 10 nM or greater, was noted; magnitude of cell death was not significantly different from incubation with Locke's buffer vehicle. A floor effect was identified at the lowest concentrations tested, 100 pM or less, with the magnitude of cell death not significantly different from that observed by the toxic synergism of the HIV proteins with cocaine.

DHT, the androgen receptor (AR) active metabolite of T, was tested for neuroprotection in the linear range of the T concentration-effect curve, 1 nM, 10 nM and 100 nM, against the toxic synergism of HIV proteins with cocaine (FIG. 17). DHT treatment was confirmed to provide significant neuroprotection (F(1,57)=10.66, p<0.002; n=at least 4 each point). However, there was no concentration-dependent effect on percent neuronal death (F(1,57)<1.0). Further, the neuroprotection was incomplete across these concentrations as the magnitude of cell death was significantly greater than that observed with Locke's Buffer control (F(1,57)=6.72, p<0.012) (FIG. 17 Bottom Panel). Because the DHT treatments were run in parallel with the T concentration effects, the control values are the same for both sets of treatments. That is, the presence of synergistic neurotoxicity was indicated by the same significant interaction of the HIV proteins with cocaine (F(1,20)=8.47, p<0.0087) as it was for the T treatments (FIG. 17 Top Panel). Ethanol at 10 nM (0.1%) served as the solvent for DHT; therefore, it was tested for toxicity and was found not to differ significantly from Locke's buffer incubation.

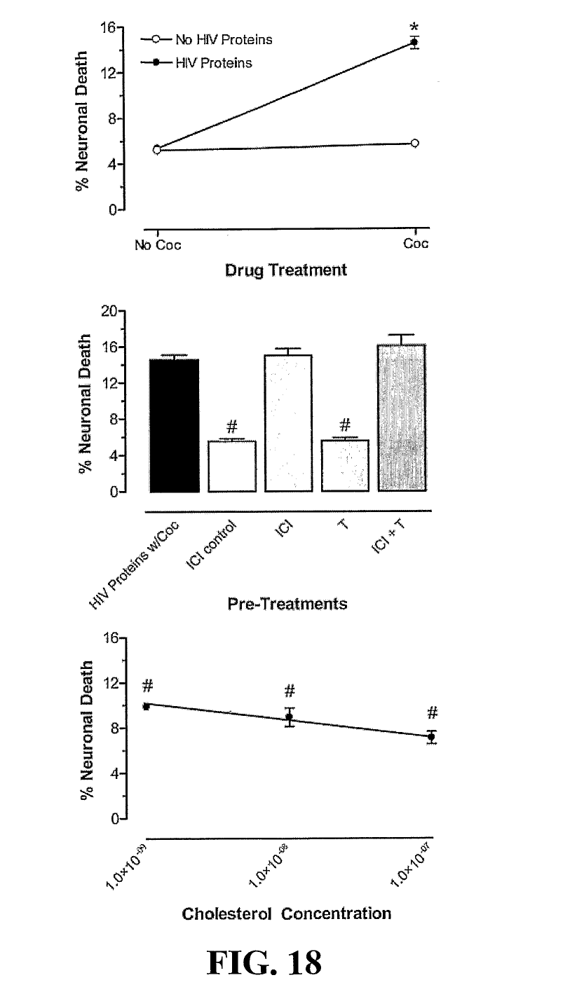

The cultures were pretreated with the specific ER antagonist, ICI-182,780 at 100 nM before addition of T (10 nM) then challenged with the HIV proteins plus cocaine synergistic toxicity (FIG. 18). A significant interaction of ICI-182,780 with T was observed (F(1,22)=33.71, p<0.0001; n=at least 6 each point). Importantly, the ICI compound was not more toxic to the cultures than was Locke's buffer; nor was it neuroprotective against the toxic challenge. T at 10 nM provided neuroprotection (F(1,65)=121.26, p<0.0001), which was not significantly different from incubation with Locke's buffer vehicle alone. This neuroprotective effect of T was fully blocked by pretreatment of the cultures with ICI-182,780 (100 nM) (FIG. 18 Middle Panel). A significant interaction of the HIV proteins with cocaine indicated the presence of synergistic neurotoxicity (F(1,28)=61.04, p<0.0001; n=8 each point) (FIG. 18 Top Panel).

Cholesterol was added to the cultures at concentrations of 1 nM, 10 nM, and 100 nM prior to treatment with the synergistic toxic combination of HIV proteins plus cocaine (FIG. 18). Significant neuroprotection by cholesterol against this synergistic toxicity was observed (F(1,65)=55.74, p<0.0001; n=at least 3 each point) (FIG. 18 Bottom Panel). There was a concentration-dependent effect of cholesterol with a significant linear decrease in percent neuronal death as concentration increased (F(1,65)=4.4042.73, p<0.0398). The neuroprotection afforded by the high dose (100 nM) of cholesterol was asymptotic to a ceiling effect; the magnitude of cell death was not significantly different from that observed by Locke's buffer incubation. Because the cholesterol treatments were performed concurrently with the ICI-182,780 treatments, the control values are the same for both sets of treatments. The presence of synergistic neurotoxicity was indicated by the same significant interaction of the HIV proteins with cocaine as it was for the ICI-182,780 treatments (F(1,28)=61.04, p<0.0001) (FIG. 18 Top Panel).

### Example 6

### Dopamine Receptor Agonist Attenuates Cocaine-Induced Enhancement of Tat Toxicity

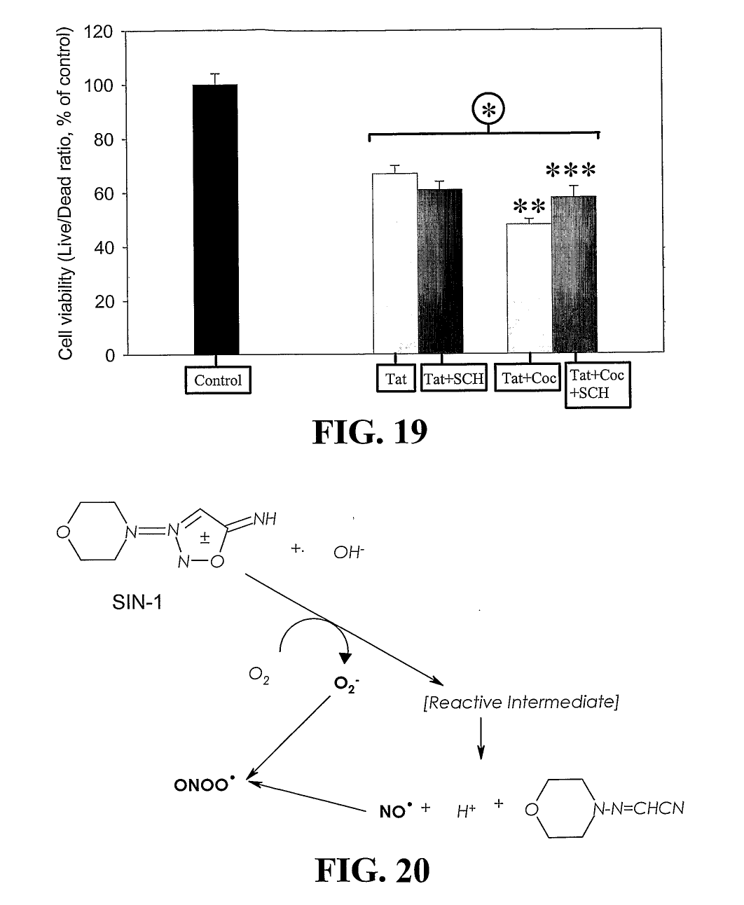

As shown in FIG. 19, doses of SCH 23390 from 0.1 to 10 μM did not cause any changes in neuronal cell viability. Consistently, when added alone to cell cultures, 10 μM SCH 23390 did not produce any changes in Live/Dead ratio compared to controls. 1.5 μM dose of cocaine did not change cell viability. Ten μM dose of SCH 23390 did not change the toxicity of Tat. However, cocaine enhanced Tat toxicity (**, Tat+cocaine versus Tat, P=0.000008). Ten μM SCH 23390 improved the survival of rat hippocampal neurons subjected to Tat+cocaine (***, Tat+cocaine versus Tat+cocaine+SCH, P=0.02). The addition of 10 μM SCH 23390 attenuated cocaine-induced enhancement of Tat toxicity in rat hippocampal cell cultures (Tat versus Tat+cocaine+SCH, no significant difference; Tat+SCH versus Tat+cocaine+SCH, no significant difference).

### Example 7

### Estrogen Attenuates gp120- and tat1-72-Induced Oxidative Stress and Prevents Loss of Dopamine Transporter Function

### Methods

Chemicals and Drugs: Cell culture media (RPMI-1640 without Glutamine), penicillin (10,000 I.U./ml)/streptomycin (10,000 (g/ml), and trypsin (0.25%)/EDTA (0.1%) solutions were obtained from Mediatech (Herndon, Va.); fetal bovine serum was obtained from Atlanta Biologicals (Norcross, Ga.). The estrogen antagonist, ICI 182,780 was obtained from Tocris Cookson (Ellisville, Mo.). SIN-1 (3-morpholinosydnonimine-peroxynitrite and superoxide) was obtained from Molecular Probes (Eugene, Oreg.). All other chemicals or drugs were obtained from Fisher Scientific (Fairlawn, N.J.) or Sigma-Aldrich (St. Louis, Mo.) and were ACS reagent- or culture-grade where appropriate. Sterile pipettes, 25 cm² flasks, and 96-well plates were obtained from Fisher Scientific (Fairlawn, N.J.).

Production of tat₁₋₇₂, tat_{Δ31-61} and gp120: Tat₁₋₇₂ was produced as previously described (Khurdayan et al., 2004; Self et al., 2003; Prendergast et al., 2002; Gurwell et al., 2001; Ma and Nath, 1997). Briefly, the tat gene encoding the first 72 amino acids (tat₁₋₇₂) was amplified from HIVBRU (AIDS repository at the NIH) and inserted into an E. coli (PinPoint Xa-2) vector (Promega, Madison Wis.). A deletion mutant from this plasmid was also prepared by deleting the sequence encoding amino acids 31-61 of tat previously shown to contain the neurotoxic epitope (Nath et al., 1996). Recombinant active tat₁₋₇₂ was prepared as previously described (Ma and Nath, 1997). Tat₁₋₇₂ proteins expressed from this construct are naturally biotinylated and can be purified on a column of soft release avidin resin, cleaved from the fusion protein using factor Xa-2, eluted, and desalted on a PD10 column. All purification steps contained dithiothreitol to prevent oxidation of the proteins. Tat proteins were >95% pure as determined by sodium dodecyl sulphate—polyacrylamide gel electrophoresis (SDS-PAGE) followed by silver staining and analysis by HPLC. Western blot analysis showed that these preparations contained both the monomeric and dimeric forms of tat₁₋₇₂, but the monomeric form only for tat_{Δ31-6}. The functional activity of tat₁₋₇₂ was confirmed using a transactivation assay in HL3T1 cells containing an HIV-1 long terminal repeat (LTR)-CAT construct (Self et al., 2003; Prendergast et al., 2002; Gurwell et al., 2001). Recombinant gp120 was produced in a Chinese hamster ovary cell line from HIVSF2 and purified to yield a product that was 95% gp120, with the remainder being breakdown products of gp120 as determined by Western Blot analysis. Both the tat and gp120 preparations contained <1 pg/ml endotoxin as determined using a Pyrochrome Chromogenic test kit (Associates of Cape Cod, Falmouth Mass.). The tat protein was stored in a lyophilized form at −80° C. in endotoxin-free siliconized tubes until used. Due to the unstable nature of the tat and gp120 proteins with repetitive freeze/thaw cycles, aliquots were made and used on the day of the experiment, with any additional protein solution being discarded.

Cell Culture Assays: SK-N-SH cells (ATCC) were grown in RPMI 1640 with 1% streptomycin/amphotericin and 10% FBS and incubated at 37° C. in the presence of 5% CO₂. Cells were subcultured into 96-well plates at a density of 10⁴ cell/well and used within 24 hours. Plates were first washed with warmed Krebs buffer (KHB). Stock DCFH in DMSO was diluted in KHB to a final concentration of 100 μM and incubated in each well for 30 minutes. DCFH dye was removed and cells were washed with warmed KHB and the appropriate drug or drug combination was added. Plates were returned to the incubator and drugs were allowed to incubate for 30 minutes. Incubation with the free radical generator SIN-1 (3-morpholinosydnonimine-peroxynitrite and superoxide) was carried out for an additional 30 minutes. The second series of experiments utilized tat (10-250 nM) or gp120 (25-100 pM) in place of the free radical generator.

The concentration of either E2 or progesterone was 0.1 to 100 nM. Plates were returned to the incubator with the hormones and were allowed to incubate for 15 minutes. SIN-1 (50 μM) was chosen as the free radical generator due to the production of peroxynitrite. Previous studies have shown that this concentration of SIN-1 yielded maximum increases in DCFH fluorescence (Wang and Joseph, 1999). In groups that examined the protective effects of E2 and/or progesterone, the hormone was added 15 minutes prior to addition of SIN-1, tat or gp120. Studies that have examined the simultaneous addition of the oxidative stress agent and hormone have yielded attenuated results. Following incubation with SIN-1, tat or gp120 (depending on the assay), fluorescence was determined by using a Bio-Tek plate reader with the settings of 485 n (excitation) and 585 nm (emission) with an integration time of 40 ms. Assays were run four separate times in triplicate. Data are expressed as mean of the separate assays±SEM.

[³H] Dopamine uptake in synaptosomes assays: Striatal tissue from 4-5 month old male Sprague-Dawley rats (N=4) was homogenized in 5 mL ice-cold 0.32 M sucrose using a glass/Teflon homogenizer followed by differential centrifugation resulting in a crude P2 preparation. Crude P2 preparations are resuspended to a final volume of 15 mL (0.13-0.15 μg protein/tube) in assay buffer (consisting of 25 mM HEPES, 120 mM NaCl, 5 mM KCl, 2.5 mM CaCl₂, 1.2 mM MgSO₄, 300 mM ascorbic acid, 1 μM pargyline and 2 mg/mL D-(+) glucose). P2 preparation was then added to assay tubes in 400 μL, followed by 50 μL of buffer or drug. Uptake was initiated by the addition of [7, 8 ³H] dopamine, 0.02 M acetic acid:ethanol (1:1) solution (Amersham Biosciences, UK). Synaptosomes were preincubated first with 100 nM E₂ 30 minutes to the initiation of uptake, followed by addition of the appropriate concentrations of gp120 (30 pM) and tat (60 nM), or both proteins, 15 minutes prior to the start of the uptake assay. Assays were then started by the addition of [3H] dopamine (20 mM, to determine “apparent Vmax”) to the synaptosome/buffer mixture for a final volume of 500 μl. To determine the effects of tat and gp120 on the kinetics of [³H] dopamine uptake, rat striatal synaptosomes were prepared as described and incubated in the presence of increasing concentrations (0-2,500 nM) of [3H] dopamine. Uptake was allowed to proceed for 20 minutes and terminated by the addition of ice-cold 0.9% NaCl and filtered under reduced pressure. Filters were washed 15 seconds with ice-cold 0.9% NaCl and total [³H] dopamine uptake was determined by scintillation spectrophotometry. Nonspecific uptake was determined in a parallel set of tubes run in the presence of 1 μM Mazindol, a dopamine uptake inhibitor. Data are expressed as the mean±SEM (N=4).

### Results

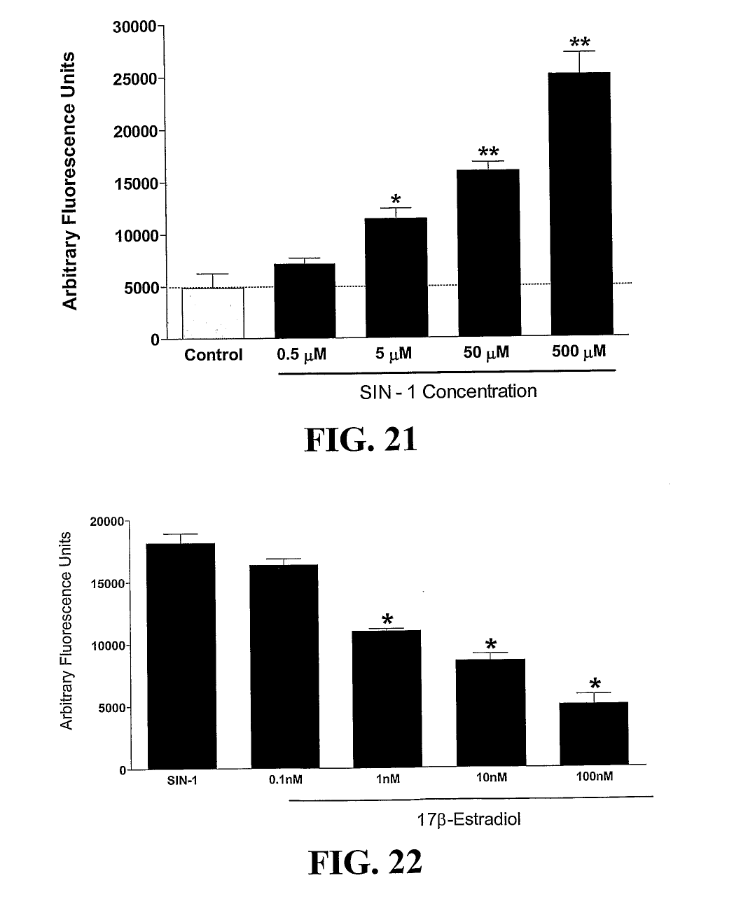

Incubation of SK-N-SH cells with SIN-1 results in the formation of superoxide and peroxynitrite radicals (FIG. 20), Increasing concentrations (0-500 μM) of SIN-1 resulted in a significant (F_{4,14}=39.79; p<0.0001) concentration-dependent increase in DCFH fluorescence in SK-N-SH cells (FIG. 21). Post-hoc comparison to control values using Dunnett's Multiple Comparison Test revealed a significant increase in fluorescence at the lowest concentration of 5 μM (q=3.642; p<0.05) followed by both the 50 μM (q=6.132; p<0.001) and 500 μM (q=11.24; p<0.001) concentrations. The concentration of 50 μM was chosen as the concentration for future studies due to the excellent signal:noise ratio and the ability to increase fluorescence greater than 3-fold over baseline (vehicle) levels.

Effect of E2 on SIN-1-induced increase in DCFH fluorescence: Preincubation with increasing concentrations of E₂ significantly (F_{4,14}=80.07; p<0.001) reduced the fluorescence induced by 50 μM SIN-1, a peroxynitrite generator (FIG. 22). Addition of 50 μM SIN-1 increased fluorescence over control values (baseline) by nearly 3.5-fold. Multiple comparisons using Student Newman-Keuls Multiple comparison test revealed that 1 nM E2 was the lowest concentration that exhibited a significant (q=11.78; p<0.001) reduction in fluorescence compared to the SIN-1 control group. Both the 10 nM (q=15.75; p<0.001) and 100 nM (q=21.63; p<0.001) groups significantly attenuated the fluorescence of the SIN-1 group (FIG. 22). In addition, as concentrations of E₂ were increased, significant reductions in fluorescence were observed compared to the previous concentration of 17β-estradiol (0.1 nM v. 1 nM, q=8.822, p<0.001; 1 nM v. 10 nM, q=3.974, p<0.05; 10 nM v. 100 nM, q=5.879, p<0.01). The highest concentration of E₂ reduced fluorescence levels to control values. Therefore, this was chosen as the experimental concentration utilized in the protection assays.

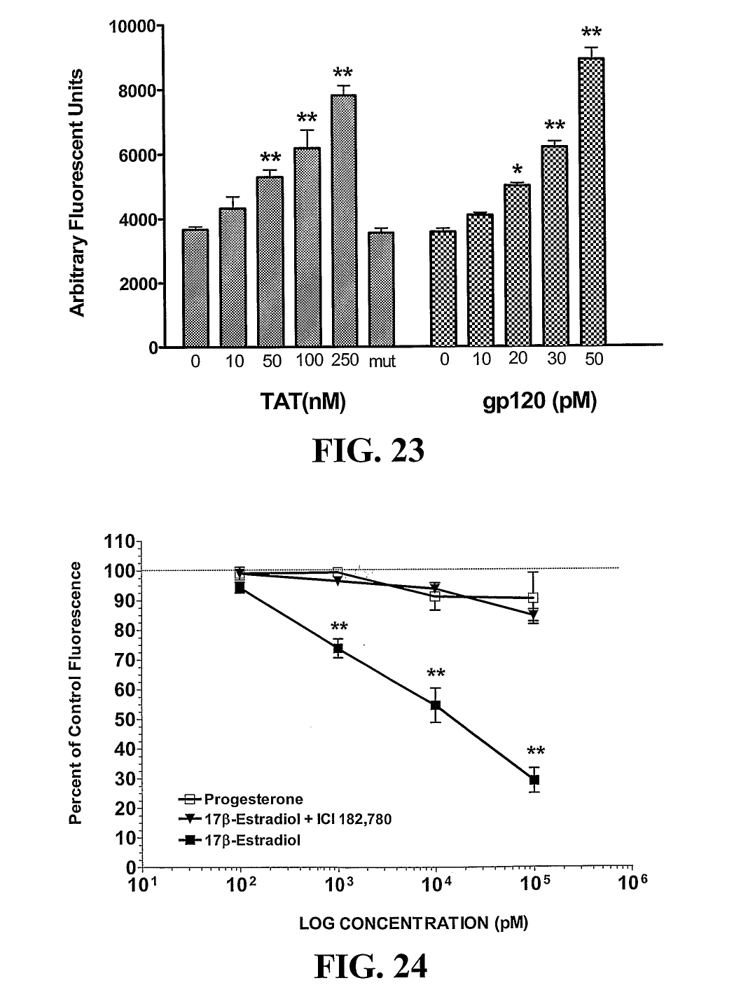

Effect of tat- and gp120 on DCFH fluorescence: HIV-1 proteins, tat (0-250 nM) and gp120 (0-100 pM) significantly increased DCFH fluorescence following 30-minute exposure. Tat significantly (F_{4,19}=21.61; p<0.0001) increased DCFH fluorescence in a concentration dependent manner. 50 nM (q=3.265; p<0.05), 100 nM (q=5.066; p<0.01) and 250 nM (q=8.378; p<0.01) were significantly greater than control (FIG. 23). To assess the ability of mutant tat (tat_{Δ31-61}) to elicit oxidative stress, a separate series of wells was incubated in the presence of 250 nM mutant tat. There was no change in DCFH fluorescence compared to control values (3555±141 and 3665±99 AFU, respectively). A similar profile was observed when SK-N-SH cells were incubated in the presence of gp120 (F_{4,19=21.61}; p<0.0001). The apparent threshold for gp120-induced fluorescence was 20 pM. Concentrations of 20, 30 and 50 pM gp120 were all significantly elevated compared to control values. The lowest concentration of gp120 (20 pM) significantly (q=4.617; p<0.05) elevated fluorescence levels compared to control (vehicle). Higher concentrations (30 pM, q=7.165; p<0.01 and 50 pM, q=11.85; p<0.01) of gp120 resulted in further increases in DCFH fluorescence up to 4.25-fold over control (vehicle) levels (FIG. 23).

Comparison between E₂ and progesterone on tat- and gp120-induced fluorescence: involvement of E₂ receptors: To examine the role of E₂ receptors in attenuating tat and gp120 oxidative stress, SK-N-SH cells were exposed to a combination of tat and gp120 (60 nM and 30 pM, respectively). The concentrations that were chosen for each protein significantly increased DCFH fluorescence approximately 50-70% compared to control values. The combination of the two proteins resulted in an additive increase 100-160% compared to control values. SK-N-SH cells were incubated in the presence of a combination of gp120 (30 pM)+tat (60 nM), which increased DCFH fluorescence, indicating oxidative stress (control values 5,736±834 RFU). To determine the effect of gonadal steroids on gp120- and tat-induced oxidative stress, a separate series of wells were co-incubated with 100 mM progesterone, 100 nM E₂, or 100 nM E₂+1 μM ICI 182,780. Two-way analysis of variance revealed a significant effect of hormone (F_{2,30}=94.71; p<0.0001), concentration (F_{4,30}=43.24; p<0.0001) and a significant interaction between hormone and concentration (F_{8,30}=13.48; p<0.0001). Post-hoc comparison with Student Newman-Keuls revealed that concentrations of 1, 10 and 100 nM E₂ were each significantly (p<0.001) different from control values (FIG. 24). Neither progesterone nor E₂+ICI 182,780 values differed significantly from control values. These data indicate that the majority of E₂ effects are due to E₂ receptors on SK-N-SH cells. A small percentage of DCFH fluorescence was still apparent at an E₂ concentration of 100 μM, suggesting that E₂ attenuation of tat+gp 120 oxidative stress is not complete, or that a small component of the E₂ effect may be mediated by a non-receptor mechanism. It also appears that these effects are selective for E₂ since progesterone had no effect on tat+gp120-induced fluorescence.

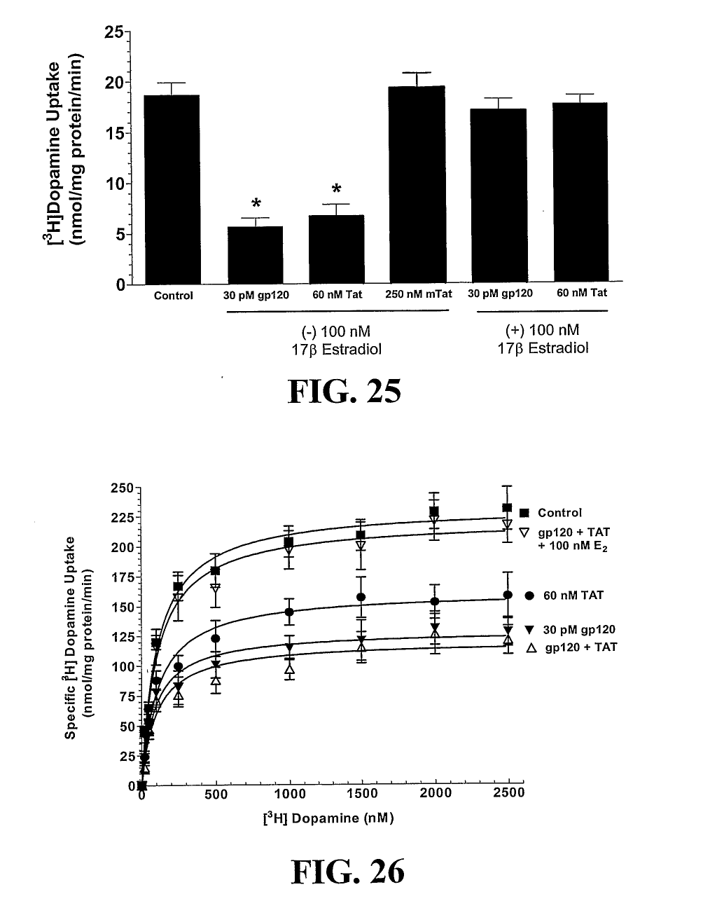

Effect of E₂ on tat or gp120 Kₘ and Vₘₐₓ values for [³H] dopamine uptake in rat striatal synaptosomes: To examine the effect of E₂ on tat- and gp120-induced alterations in [³H] dopamine uptake in the rat, striatal synaptosomes were incubated in the presence of either 60 nM tat or 30 pM gp120 in the absence and presence of 100 nM E₂ (FIG. 25). There was a significant (F_{4,14=35.41}; p<0.0001) effect of treatment combination. To further investigate the effects of mutant tat_{Δ31-61} on [³H] dopamine uptake, a separate series of synaptosomal preparations were incubated with 250 nM tat_{Δ31-61}. Incubation did not affect [³H] dopamine uptake if compared to control values (19.3±1.5 and 18.7±1.2 mmole/mg protein/minute, respectively). Post-hoc comparison using Dunnett's Multiple Comparison Test to compare fluorescent values in treatment groups to control revealed both gp120 (q=8.551; p<0.01) and tat (q=7.894; p<0.01) significantly reduced [³H] dopamine uptake by >60% compared to control values. Co-incubation with 100 nM E₂ reversed this reduction and [³H] dopamine uptake into rat striatal synaptosomes was not different from controls in either the gp120 (q=1.096; p>0.05) or tat (q=0.658; p>0.05) groups. Studies which examined the preservation of [3H] dopamine uptake demonstrated that the highest concentration of E2 used in the previous studies could reverse the reduction in apparent Vmax previously observed when synaptosomes were incubated in the presence of either 30 pM gp120 or 60 nM Tat. Kinetic analysis of [³H] dopamine uptake in rat striatal synaptosomal preparations was determined in the presence gp120 (30 pM), tat (60 nM), alone or in combination, in the absence or presence of 100 nM E₂ (FIG. 26). Uptake curves were best fit to a single-site rectangular hyperbola with Kₘ values of 90-109 nmol/mg protein/min (Table 1). None of these Kₘ values reached statistical significance compared to control values. A significant (F_{4,19}=480.9; p<0.0001) effect of treatment on Vₘₐₓ values was observed. Post-hoc analysis with Student Newman-Keuls revealed a significant reduction (30.5%-48.5%) in Vₘₐₓ values for gp120 (q=43.80; p<0.001) and tat (q=29.93; p<0.001) alone as well as in combination (q=47.56; p<0.001,). The effects of gp120 and tat lower concentrations of [³H] dopamine were essentially reversed by co-incubation with 100 nM E₂. Complete kinetic analysis revealed that this attenuation of dopamine uptake was due to a reduction in Vₘₐₓ and not a decline in affinity, or Kₘ. The reversal of gp120 and tat effects was not complete, although significant (q==4.692; 231 nmol/mg protein/minute compared to 220 nmol/mg protein/minute), in comparison to 30-48% reductions observed in the tat and gp120 groups.

TABLE 1
Summary of Kₘ and Vₘₐₓ values for [³H] dopamine uptake in striatal
synaptosomes following incubation with 30 pM gp120, 60 nM tat,
combination of gp120/tat and a combination of gp120/tat + 100
nM E₂.

| | Control | gp120 | tat | gp120 + tat | gp120 + tat + E₂ |
|---|---|---|---|---|---|
| Kₘ | 106.1 ± 5.4 | 90.0 ± 6.9 | 109.2 ± 6.8 | 98.9 ± 10.9 | 107.9 ± 6.4 |
| Vₘₐₓ | 231.9 ± 2.4 | 128.3 ± 1.9** | 161.1 ± 2.1** | 119.4 ± 2.6** | 220.8 ± 2.7* |

Kₘ values are expressed as nM.
Vₘₐₓ values are expressed as mean nmoles [³H] dopamine uptake per milligram protein over 20 minutes.
All data are expressed as mean ± S.E.M of 4 experiments assayed in duplicate.
**p < 0.001 compared to control Vₘₐₓ values.
*p < 0.05 compared to control Vₘₐₓ values.

### Example 8

### Neonatal Hippocampal Tat Injections: Developmental Effects on Prepulse Inhibition of the Auditory Startle Response

### Methods

Animals: Sprague-Dawley pregnant dams (n=8) were obtained from Harlan Laboratories, Inc. (Indianapolis, Ind.) and delivered to the vivarium before embryonic day seven. Dams were housed singly with food (Pro-Lab Rat, Mouse Hamster Chow #3000, NIH diet #31) and water available ad libitum. The day pups were found in the cage was designated as postnatal day (P) 0. On P1, litters were culled to 10 offspring of equal sexes, if possible. No more than one female and one male per litter were assigned to a single condition. The animal facility was maintained at 21±2° C., 50%±10% relative humidity and had a 12-hour light: 12-hour dark cycle with lights on at 0700 h (EST). The animals were maintained according to the National Institute of Health (NIH) guidelines in AAALAC-accredited facilities.

Surgery: Individual pups were gently removed from the dam and immersed in ice anesthesia before being placed in a modified stereotaxic holder for surgery. Rubber head bars held the skull in place while bilateral microinjections (1 μl) of saline or (50 μg) of Tat were made directly into hippocampus using the following set of coordinates: right hemisphere −0.3 mm AP, 0.7 mm ML, −3.0 mm DV; left hemisphere −0.3 mm AP, −0.7 mm ML, −3.0 mm DV. The 1 μl injection volume was released over one minute after a one-minute resting period that allowed the tissue to return to its original conformation. The needle was withdrawn over 2 minutes to prevent reflux. After the two injections, the piercings in the skin of the head were closed with Dermabond and the pups warmed under a heat lamp (35° C.) before being returned to the dam, where they were closely observed for indications of rejection. No pups were rejected or abused by the dam.

Experimental Design: Male and female rats were randomly assigned to one of two treatments that received bilateral hippocampal injections of either 1 μl volume saline (nₘₐₗₑₛ=8, n_{females}=6) or 50 μg Tat (nₘₐₗₑₛ=8, Tat n_{females}=6). All rats were tested for PPI of the acoustic startle response (ASR) at 30, 60, and 90 days of age. To control for repeated testing, a separate adult group was tested only once, at 90 days of age, on PPI of the ASR as described above. Male and female rats were randomly assigned to a saline-treated group (nₘₐₗₑₛ=6, n_{females}=8), or a Tat-treated group (nₘₐₗₑₛ=7, n_{females}=6). The surgical procedure was equivalent the one used for the longitudinal tested group.

Apparatus: The startle chamber (SR-Lab Startle Reflex System, San Diego Instruments, Inc.) was enclosed in an 81×81×116-cm isolation cabinet (Industrial Acoustic Company, INC., Bronx, N.Y.). Each animal was tested individually in the dark with a high-frequency loudspeaker, mounted inside the chamber 31 cm above the Plexiglas cylinder that produced a background white noise (70 dB(A)). The startle chamber for animals at 30 days of age consisted of a Plexiglas cylinder 8.75 cm in interval diameter resting on a 12.5×20-cm Plexiglas stand. The animal's response to the stimulus produced deflection of the Plexiglas cylinder, which was converted into analog signals by a piezoelectric accelerometer. Acoustic stimulus intensities and response sensitivities were calibrated using a SR-LAB Startle Calibration System. Sound levels were measured and calibrated with a sound level meter (Quest electronics: Oconomowoc, Wis.) with microphone placed inside the Plexiglas cylinder. The signals were then digitized (12 bit A to D) and saved to a hard disk.

Testing Procedures: All rats were tested approximately 20 min. Animals were first exposed to a 5-minute acclimation period of 70 dB(A) background, followed by six single stimuli, and 36 PPI trials with 0, 8, 40, 80, 120, and 4000 msec interstimulus interval (ISI), assigned by Latin-square design. The six single stimuli were defined as adaptation trials and the PPI trials 0 and 4000 msec were the control trials in order to control for PPI. The prepulse stimulus intensity was 85 dB(A). For PPI the dependent measures analyzed were peak ASR magnitude, peak ASR latency, and percent PPI. Percent PPI indicates the percent of inhibition in startle magnitude at a prepulse of 100 msec ISI relative to pulse only trials (0 msec ISI). PPI for ISI 100 msec was calculated using the average of PPI trials 80 and 120 msec ISIs. Percent PPI was computed according to the following formula: % PPI=[(0 msec ISI trials −100 msec ISI trials)/0 msec ISI trials]*100.

Histology: After approximately 8 month of age, animals were euthanized (pentobarbital overdose) and hippocampal brain tissue collected. Cryostat-cut sections (20 μm) through the hippocampal injection sites were collected and Nissl-stained confirmed injection location and pathology.

Statistical Analysis: All data were analyzed using analysis of variance (ANOVA) techniques (SPSS, 2003; SYSTAT, 2003; Winer, 1971). For the longitudinal tested animal group three-way mixed-factor ANOVAs, with treatment and sex as between-subjects factors and age as a within-subjects factor were performed on peak ASR magnitude and peak ASR latency in the adaptation trials, control trials and on percent PPI (through 100 msec). A four-way mixed-factor ANOVA, with treatment and sex as between-subjects factors and PPI trials and age as within-subjects factors were performed on peak ASR magnitude and peak ASR latency across PPI trials (8-120 msec). ANOVAs were conducted in order to characterize the ISI function in PPI and to determine whether intrahippocampal administration of Tat on PI altered PPI across different age groups. ANOVAs for the peak magnitude for response inhibition across PPI trials (8-120 msec ISI) were conducted on log 10 transformed data and log 10 metric for ISI. ANOVAs for the latency response across PPI trials (8-120 msec ISI) were conducted on raw data with the original metric for ISI [8, 40, 80, 120]. The Greenhouse-Geisser df correction factor and contrast analyses were employed for violations of sphericity in repeated measures. To correct for violations in sphericity, contrast analyses for main effects and interactions were used in order to avoid analyses that violated the homogeneity of variance assumption (Winer, 1971). Trend analyses for simple effects of PPI trials separately for each treatment were employed to evaluate the nature of the PPI trial-dependent effects. This statistic also describes the shape of a trend by determining its significance (e.g., linear, quadratic, etc. trends). Trend analyses for simple effects of PPI trials were further analyzed when appropriate. In addition, ISI in which the peak occurred was recorded across all PPI trials (8-120 msec ISI). The ISI data is categorical in nature, thus, the Pearson chi-square, uncorrected for continuity was applied. For the adult group that was tested only once, at 90 days of age, analyses were the same with the exception that age was not included as a factor. A two-way multivariate analysis of variance (MANOVA), with treatment and sex as between-subjects factors was performed on hippocampal cell density [CA1, CA3, dentate gyrus, pyramidal, granule] combining left and right hemisphere. No group differences across both experimental groups were noted, thus, data of animals tested repeatedly and animals tested only once were combined and analyzed together. An alpha level of p<0.05 was considered significant for all statistical tests used.

### Results

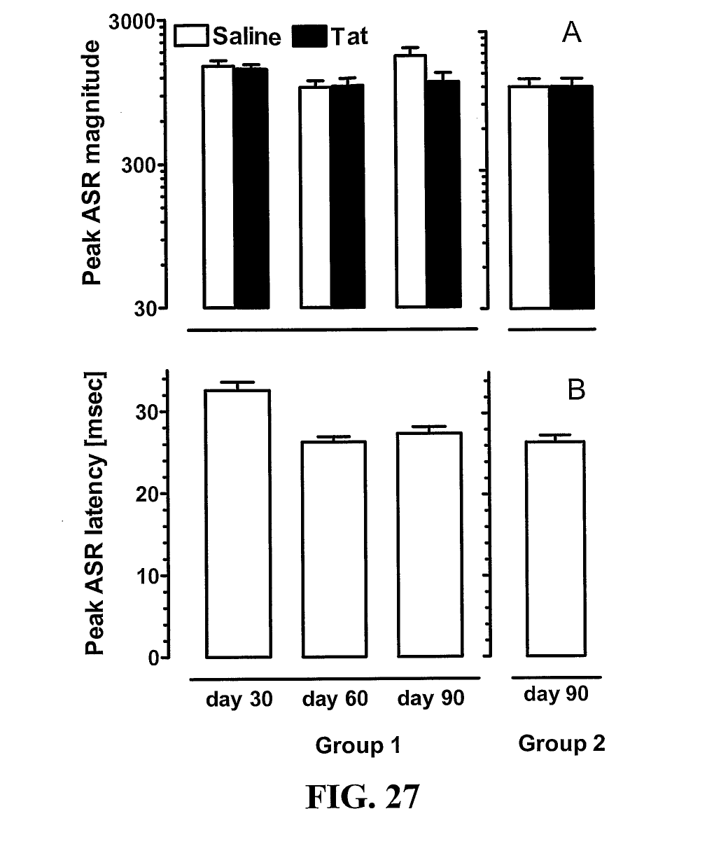

A 2(treatment)×2(sex)×3 (age 30, 60, 90 days old) mixed-model ANOVA conducted on peak ASR magnitude revealed a significant age effect with a quadratic trend [F(1, 23)=18.20, p<0.001] and an treatment×age interaction [F(2, 46)=3.30, p=0.046]. Separate trend analyses for treatment revealed a significant quadratic trend for saline-treated rats [F(1, 11)=21.89, p 0.001]. In contrast, no significant trend was noted for Tat-treated animals, indicating an alteration of the peak response curve on adaptation trials across age. A mixed-model ANOVA on peak ASR latency revealed a significant age effect with a prominent linear trend [F(1, 23)=17.66, p<0.001]. For the adult group, tested only at 90 days of age, no significant effects on peak magnitude and peak latency were noted. Peak ASR magnitude is illustrated in FIG. 27a. Peak ASR latency is illustrated in FIG. 27b.

Prepulse inhibition (PPI) Test: One saline-treated male had to be excluded in PPI assessment at 30 days of age that did not show any sensitivity to the PPI trials.

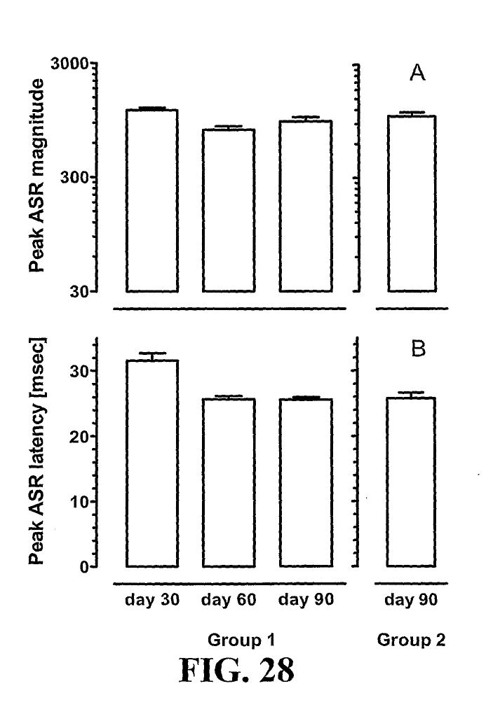

Control Trials (0 and 4000 msec combined): A 2(treatment)×2(sex)×3(age: 30, 60, 90 days old) mixed-model ANOVA conducted on peak ASR magnitude revealed a significant age effect with a prominent quadratic trend [F(1, 23)=37.44, p<0.001], indicating an alteration in peak magnitude with age. A mixed-model ANOVA conducted on peak ASR latency revealed a significant age effect with a prominent linear trend [F(1, 23)=24.27, p<0.001]. For the adult group no significant effects on peak magnitude and peak latency were noted. Peak ASR magnitude is illustrated in FIG. 28a. Peak ASR latency is illustrated in FIG. 28b.

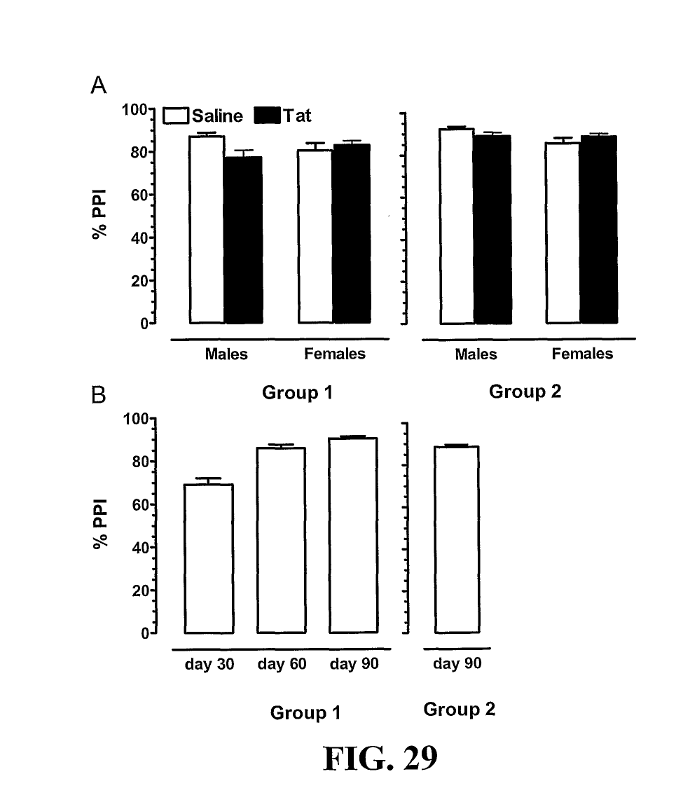

PPI trials at 8-120 msec ISI: A 2(treatment)×2(sex)×3(age: 30, 60, 90 days old)×4(PPI: 8, 40, 80, 120 msec ISI) mixed-model ANOVA conducted on peak ASR magnitude revealed a significant age effect with a prominent linear trend [F(1, 23)=132.67, p<0.001], indicating a decrease in peak response with age. For PPI trial a significant main effect was noted with a prominent linear trend [F(1, 23)=75.13, p<0.001], which was significantly altered by sex, revealing a linear sex×PPI trial interaction [F(1, 23)=4.61, p=0.043], and by age, revealing a linear age×PPI interaction [F(1, 23)=7.28, p=0.013]. Further, a three-way treatment×sex×PPI interaction revealed a tendency to a significant linear trend [F(1, 23)=3.47, p=0.075], suggesting that Tat treatment altered PPI differently for males and females. Trend analyses were conducted separately for treatment and age within each sex. At 30 and 60 days of age saline-treated males revealed prominent linear trends [30 days of age: F(1, 6)=15.23, p=0.008; 60 days of age: F(1, 7)=57.21, p=0.001], in contrast to prominent quadratic trends for Tat-treated animals [30 days of age: F(1, 7)=26.46, p=0.001; 60 days of age: F(1, 7)=9.64, p=0.017]. A leftward peak shift by Tat treatment across all PPI trials (8-120 msec) was further noted at 30 days of age [χ²(1)=4.67, p=0.031], but was only suggested at 60 days of age [χ²(1)=2.62, p<0.106]. At 90 days of age both male groups, saline- and Tat-treated males exhibited prominent linear trends [F(1, 7)=85.55, p<0.001 and F(1, 7)=16.92, p=0.004, respectively]. No peak shift was noted at 90 days of age. For females, trend analyses revealed no significant differences for treatment and no significant peak shifts on ISIs. For the adult group, a 2(treatment)×2(sex)×4(PPI: 8, 40, 80, 120 msec ISI) mixed-model ANOVA conducted on peak ASR magnitude revealed a prominent linear PPI trend [F(1, 23)=128.00, p<0.001], and a significant quadratic sex×PPI effect [F(1, 23)=5.15, p=0.033]. The two-way treatment×PPI interaction revealed a tendency to significance [F(1, 23)=3.56, p=0.072]. Separate trend analyses were conducted for the two treatment groups within each sex. Saline-treated males revealed a linear trend [F(1, 5)=22.37, p=0.005]. Tat-treated males exhibited in addition to a linear trend, a significant prominent quadratic trend [F(1, 7)=27.78, p=0.002]. However, no significant peak shift by Tat across all PPI trials (8-120 msec) was noted [χ²(1)=0.93, p=0.335]. For females trend and peak shift analyses revealed no significant effects. FIG. 29 illustrates the peak ASR magnitude for males across PPI trials (8-120 msec ISI) separate for treatment and age.

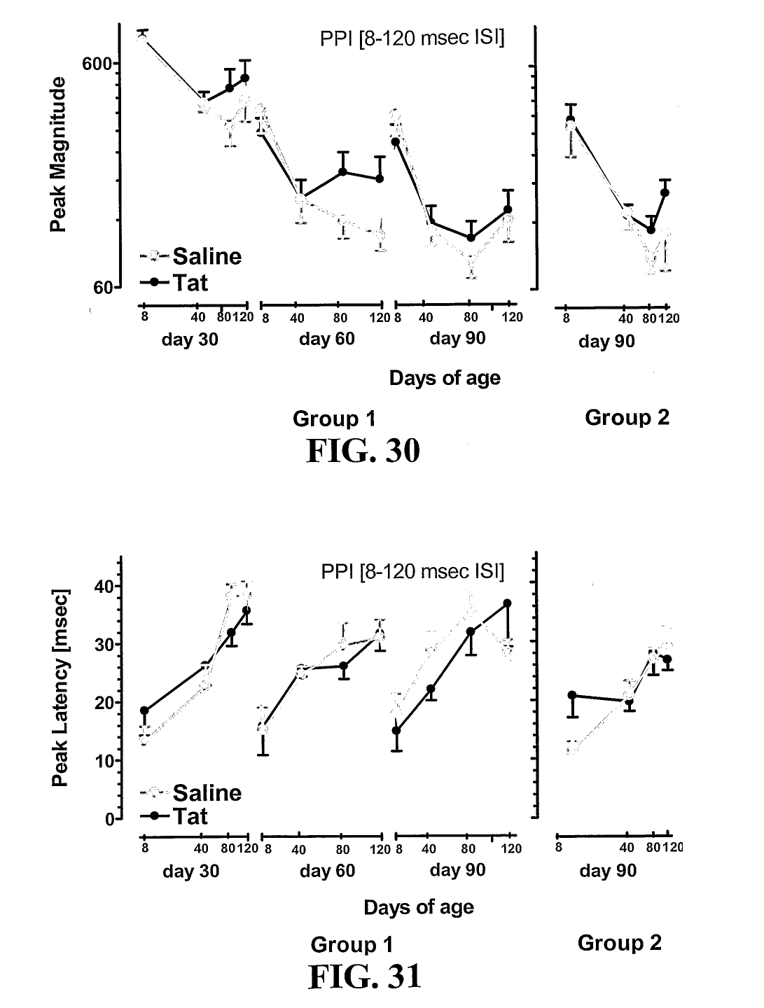

Peak ASR latency: A 2(treatment)×2(sex)×3(age: 30, 60, 90 days of age)×4(PPI: 8, 40, 80, 120 msec ISI) mixed-model ANOVA conducted on peak ASR latency [Mauchly's W=0.76, p=0.052 and 0.102, p=0.001 for age and age×PPI trial, respectively] revealed significant main effects for sex [F(1, 23)=4.24, p=0.051], a prominent quadratic age trend [F(1, 23)=15.54, p=0.001], and a prominent linear PPI trend [F(1, 23)=375.27, p<0.001]. The two-way analyses revealed a significant linear sex×PPI interaction [F(1, 23)=4.74, p=0.040], a linear age×PPI interaction [F(1, 23)=5.762, p=0.025], that were significantly altered by treatment, revealing a significant linear four-way treatment×sex×age×PPI interaction [F(1, 23)=8.89, p=0.007]. Separate trend analyses were conducted for both treatment groups within each age and sex. For males prominent linear trends at all three days of age were noted for saline-treated rats [30 days of age: F(1, 7)=172.82, p<0.001; 60 days of age: F(1, 7)=62.74, p<0.001; 90 days of age: F(1, 7)=17.73, p=0.010] as well as Tat-treated animals [30 days of age: F(1, 7)=41.03, p<0.001; 60 days of age: F(1, 7)=18.36, p=0.004; 90 days of age: F(1, 7)=20.02, p=0.003]. Planned contrasts on the linear trends revealed no significant treatment×PPI interactions for any age group [for all three age groups: F(1, 14)<1.0]. For females, 30 and 60 days old saline-treated animals revealed in addition to prominent linear trends, quadratic trends [30 days of age: F(1, 5)=6.994, p=0.046; 60 days of age: F(1, 5)=9.945, p=0.025]. In contrast, Tat-treated females revealed only linear trends [30 days of age: F(1, 5)=32.32, p=0.021; 60 days of age: F(1, 5)=10.91, p=0.021]. Planned contrasts on the linear trends revealed significant treatment×PPI interactions for 30 days old rats [F(1, 10)=4.90, p=0.051], but not for 60 days old rats. At 90 days of age, saline-treated females exhibited a prominent quadratic trend [F(1, 5)=22.08, p=0.005], in contrast to an only linear trend for Tat-treated females [F(1, 5)=18.90, p=0.007]. Planned contrast on the linear trends revealed no differences. For the adult group, a 2(treatment)×2(sex)×4(PPI: 8, 40, 80, 120 msec ISI) mixed-model ANOVA conducted on peak ASR latency revealed a prominent linear PPI trend [F(1, 23)=122.41, p<0.001], and a significant sex×PPI interaction [F(3, 69)=3.54, p=0.019]. Separate trend analyses were conducted for treatment within each sex. No trend differences and significant peak shifts were noted for males. Saline-treated females revealed a linear trend [F(1, 7)=25.839, p=0.001], in contrast to Tat-treated females that did not show any significant trends. FIG. 30 illustrates the peak ASR latency for females across PPI trials (8-120 msec ISI) separate for treatment and age.

Percent PPI: A 2(treatment)×2(sex)×3(age: 30, 60, 90 days of age) mixed-model ANOVA conducted on PPI of the startle magnitude for ISI 100 msec [Mauchly's W=0.41, p<0.001] revealed a prominent linear age effect [F(1, 23)=50.14, p<0.001]. A significant treatment×sex interaction was noted [F(1, 23)=4.71, p=0.041], that was due to a Tat-induced significant reduction in inhibition for males [F(1, 23)=6.80, p=0.016] but not for females [F(1, 23)<1.0]. Planned comparisons revealed For the adult group, a tendency to a significant sex effect was noted [F(1, 23)=3.51, p=0.074]. Percent PPI is illustrated in FIG. 31.

Analysis of Nissl-stained sections through the hippocampus confirmed placement of injection sites into the hippocampal dentate region. Saline-injected animals displayed little pathology, whereas in Tat-injected animals damage to the CA1, CA3 and dentate region was noted.

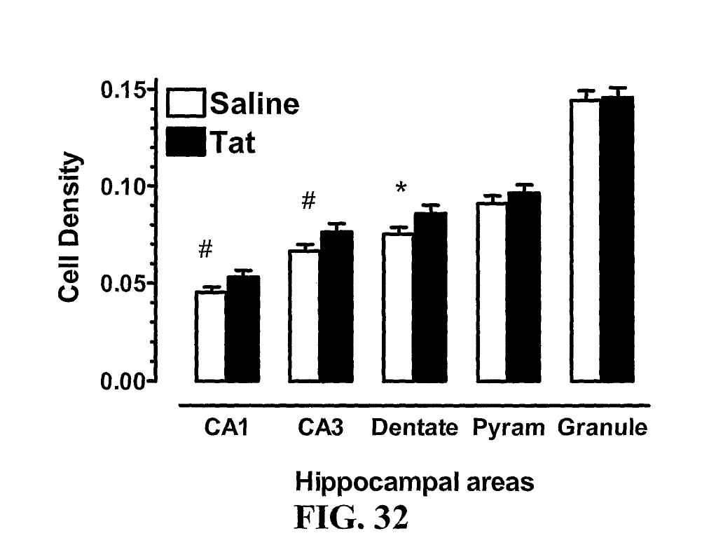

MANOVA conducted on hippocampal cell density revealed significant tendencies of a treatment effect with higher cell density for Tat-treated animals in CA1 [F(1, 46)=3.22, p=0.079], CA3 [F(1, 46)=3.25, p=0.078], and dentate gyrus [F(1, 46)=3.94, p=0.053]. In addition, the dentate gyrus revealed a significant sex effect [F(1, 46)=6.48, p=0.014]. Females showed less cell density compared to males (data not shown). FIG. 32 illustrates cell density of the five hippocampal regions separate for saline and Tat-treated animals.

### Example 9

### Intracerebral Hippocampal Injections of Tat: Effects on Prepulse Inhibition of the Auditory Startle Response

### Methods

Animals: Sixteen male Sprague-Dawley rats (˜120 days of age) obtained from Harlan Sprague Dawley, Inc. (Indianapolis, Ind.), were pair housed throughout the experiment. Rodent food (Pro-Lab Rat, Mouse Hamster Chow #3000, NIH diet #31) and water were available ad libitum. The animals were maintained according to NIH guidelines in AAALAC-accredited facilities. The animal facility was maintained at 210°±2° C., 50%±10% relative humidity and had a 12-hour light: 12-hour dark cycle with lights on at 0700 h (EST). Rats were handeled prior to any procedures to minimize stress during behavioral testing.

Experimental Design: Rats were randomly assigned to one of two treatment groups that received bilateral hippocampal injections of 1 μl volume saline (n=8) or 50 μg Tat (n=8). One Tat-treated animal died during surgery. Following three weeks recovery from surgery animals were tested in an ASR test for habituation, and a PPI test that included adaptation, control, and PPI trials. Since no Tat effects were found in the initial PPI test, animals were tested one month later (at ˜170 days of age). Habituation data collected at 140 days of age and data of the PPI test administered at 170 days of age are reported.

Surgery: Animals were anesthetized with a mixture of ketamine (100 mg/kg/ml) and xylazine (3.3 mg/kg/ml) and placed in the KOPF stereotaxic instrument. A midline sagittal incision was made in the scalp. Holes were drilled at three sites on the skull using the following set of coordinates: −3.5 mm AP, 1.4 mm ML, −3.8 mm DV; −5.9 mm AP, 3.3 mm ML, −3.8 mm DV; and −6.2 mm AP, 4.6 mm ML, −7.7 mm DV. The 1 μl injection volume was released over one minute after a one-minute resting period that allowed the tissue to return to its original conformation. The needle was withdrawn over 2 minutes to prevent reflux. Tat had no significant effect on body weight or growth.

Histology: Following completion of behavioral testing, animals were sacrified (pentobarbital overdose) and brain tissue collected. Cryostat-cut sections (20 μm) through the hippocampal injection sites were collected and Nissl-stained to confirm injections and pathology.

Apparatus: The startle chamber (SR-Lab Startle Reflex System, San Diego Instruments, Inc.) was enclosed in an 81×81×116-cm isolation cabinet (Industrial Acoustic Company, INC., Bronx, N.Y.). Each animal was tested individually in the dark with a high-frequency loudspeaker, mounted inside the chamber 31 cm above the Plexiglas cylinder, that produced a background white noise (70 dB(A)). The startle chamber consisted of a Plexiglas cylinder 8.7 cm in interval diameter resting on a 12.5×20-cm Plexiglas stand. The animal's response to the stimulus produces deflection of the Plexiglas cylinder, which is converted into analog signals by a piezoelectric accelerometer. Acoustic stimulus intensities and response sensitivities were calibrated using a SR-LAB Startle Calibration System. Sound levels were measured and calibrated with a sound level meter (Quest electronics: Oconomowoc, Wis.), A scale, with microphone placed inside the Plexiglas cylinder. The signals were then digitized (12 bit A to D) and saved to a hard disk.

ASR habituation test: In order to obtain an adequate level of habituation adult rats were exposed to a 36-trial session. Rats were placed in the startle chamber and exposed to 5 min of 70 dB(A) background noise followed by 36 pulse trials of a 100 dB(A) white noise stimulus with a 20 msec duration, according to a fixed 10 see ISI. The primary measure of the reflex response is the ‘peak’. The peak is reported in terms of its latency to the maximum response (peak), and its maximum amplitude. Thus, the dependent measures included peak ASR magnitude, and peak ASR latency.

PPI test: One month following ASR habituation assessment all adult rats were tested for approximately 20 min. Animals were first exposed to a 5-minute acclimation period of 70 dB(A) background, followed by six single stimuli, and 36 trials with 0, 8, 40, 80, 120, and 4000 msec ISI, assigned by Latin-square design. The six single stimuli served as adaptation trials to the startle stimulus and the trials 0 and 4000 msec ISI were control trials in order to control for ASR within the PPI test. The prepulse stimulus intensity was 85 dB(A). For PPI the dependent measures analyzed were peak ASR magnitude, peak ASR latency, and percent PPI. Percent PPI indicates the percent of inhibition in startle magnitude at a prepulse of 100 msec ISI relative to pulse only trials (0 msec ISI). PPI for ISI 100 msec was calculated using the average of PPI trials 80 and 120 msec ISIs. Percent PPI was computed according to the following formula: % PPI=[(0 msec IST trials −100 msec ISI trials)/0 msec ISI trials]*100.

Statistical Analysis: All data were analyzed using analysis of variance (ANOVA) techniques [33, 34, 36]. A one-way ANOVA, with treatment as a between-subjects factor was conducted for the ASR adaptation, and ASR control trials, and percent PPI (at 100 msec). A two-way mixed ANOVA, with treatment as a between-subjects factor, and trial as a within-subjects factor, was conducted on ASR habituation trials and all PPI trials (8-120 msec). The Greenhouse-Geisser df correction factor and contrast analyses were employed for violations of sphericity in repeated measures. In the present study, sphericity was violated in the mixed ANOVA for peak ASR magnitude (Mauchly's W=0.014, p<0.001). To correct for violations in sphericity, contrast analyses for main effects and interactions were used in order to avoid analyses based on a violation of homogeneity of variance [36]. Separate trend analyses for simple effects of PPI trials were employed for each treatment to determine the shape of the ISI function by its significance (e.g., linear, quadratic trends). Trend analyses for simple effects of PPI trials were further analyzed when appropriate. In addition, for peak response the ISI in which the peak occurred was recorded across all PPI trials. The ISI data is categorical in nature, thus, the Pearson chi-square, uncorrected for continuity was applied. An alpha level of p<0.05 was considered significant for all statistical tests used.

Results

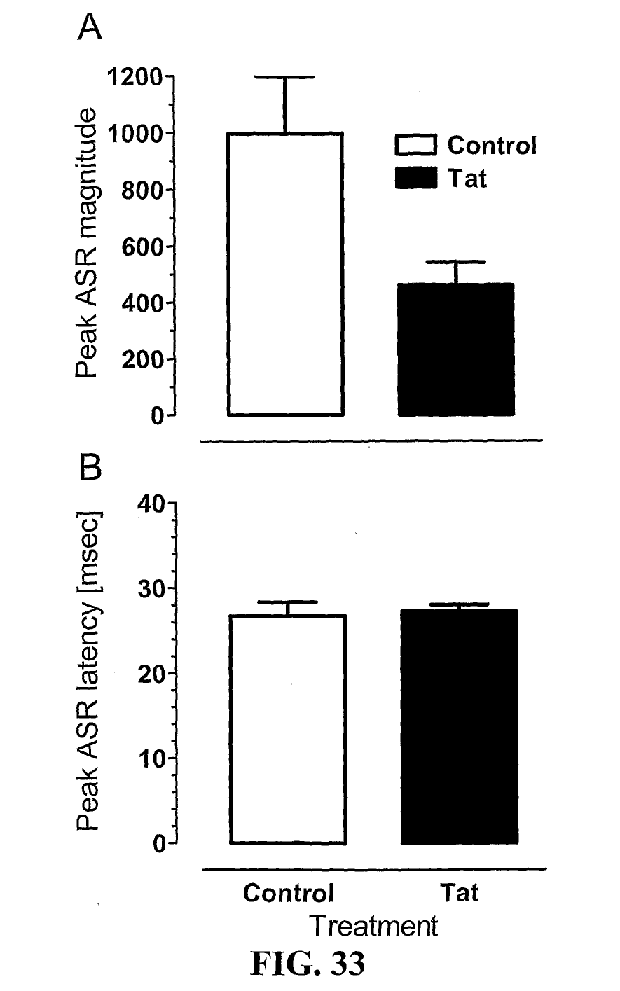

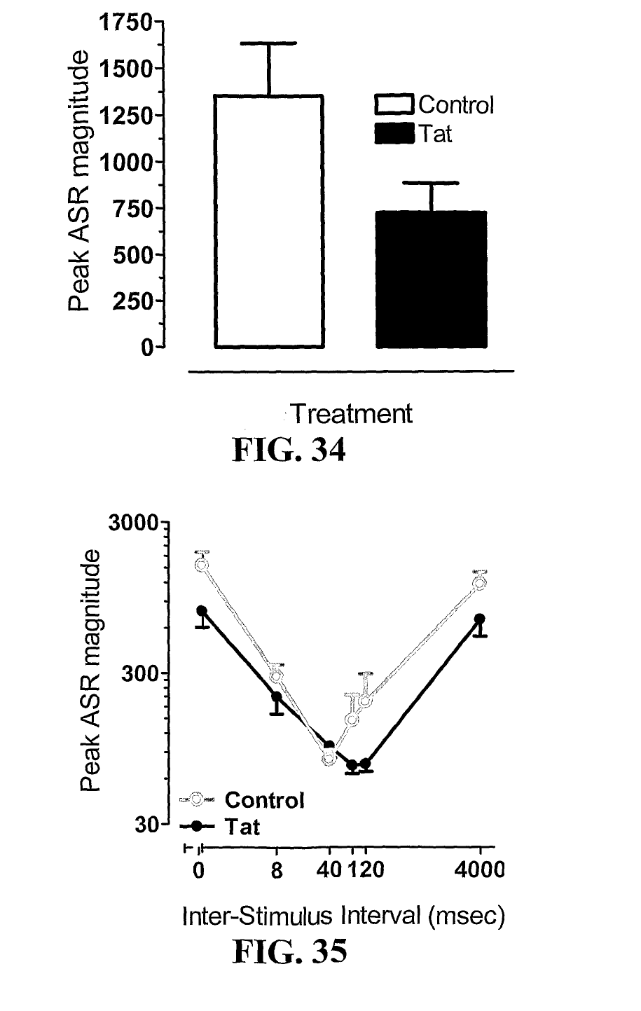

ASR habituation test: One Tat animal was excluded that did not show any sensitivity to the 36 habituation trials. A mixed-model ANOVA on peak ASR latency revealed a significant trial effect, F(35, 385)=2.11, p<0.001 [F(1, 11)=5.42, p=0.040, cubic], no treatment effect [F(1, 11)<1.0], and no trial×treatment interaction [F(35, 385)<1.0], indicating equivalent habituation rates for saline and Tat-treated rats. A mixed-model ANOVA on peak ASR magnitude revealed a significant trial effect, F(35, 385)=5.13, p_{GG}=0.001 [F(1, 11)=22.02, p=0.001, linear; F(1, 11)=16.41, p=0.002, quadratic], and a significant treatment effect, F(1, 11)=5.45, p=0.040, but no trial×treatment interaction [F(35, 385)<1.0]. Tat produced a significant attenuation of the overall peak ASR magnitude, but does not affect the rate of habituation. FIG. 33a illustrates the significant reduction in peak ASR magnitude of ˜50%, FIG. 33b illustrates the treatment effect for peak ASR latency across all 36 habituation trials in Tat-treated animals.

Adaptation Trials: A one-way ANOVA on peak ASR latency and peak ASR magnitude revealed no treatment effect [F(1, 12)<1.0 and F(1, 12)=1.40, p=0.260, respectively], indicating equivalent rates of adaptation to the startle stimulus. However, for mean (±SEM) peak ASR magnitude an attenuated response of ˜35% was noted in Tat-treated animals (972.60±271.17) compared to saline-treated rats (1461.67±313.00).

Control Trials (0, 4000 msec ISI combined): A one-way ANOVA on peak ASR latency revealed no treatment effect [F(1, 12)<1.0], indicating equivalent ASR. On peak ASR magnitude a tenedncy to a significant treatment effect was noted, F(1, 12)=3.74, p=0.077, with an 45% reduction of magnitude response in Tat-treated animals (723.05±158.86) compared to saline-treated rats (1352.83±284.19).

Percent PPI: A one-way ANOVA conducted on percent PPI of startle magnitude for ISI 100 msec revealed no treatment effect [F(1, 12)<1.0].

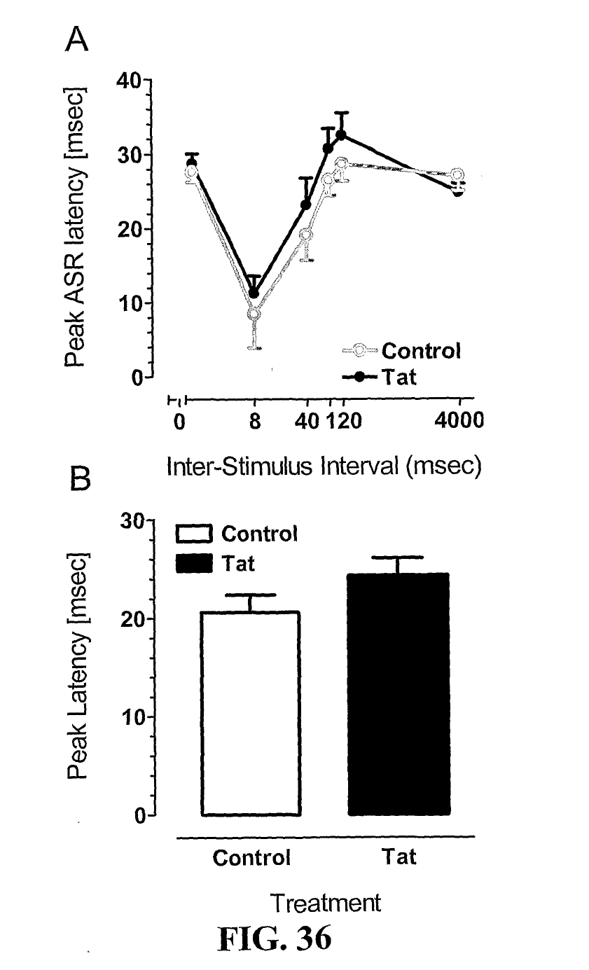

PPI trials [8-120 msec]: A mixed-model ANOVA conducted on peak ASR magnitude revealed a significant PPI trial effect with a prominent quadrantic trend [F(1, 12)=18.93, p=0.001], but no effect for treatment [F(1, 12)<1.0]. The treatment×PPI trial interaction was significant [F(1, 12)=7.84, p=0.016, quadratic]. Separate trend analyses for both treatment groups reavealed a significant quadratic trend for saline-treated rats [F(1, 6)=16.06, p=0.007], in contrast to a linear trend for Tat-treated animals [F(1, 6)=13.66, p=0.010]. FIG. 35 illustrates the peak ASR magnitude across ISIs (0-4000 msec). A rightward peak shift by Tat across all PPI trials (8-120 msec) was noted [χ²(1)=4.67, p=0.031]. The rightward peak shift and significant alteration of peak ASR function indicates a Tat-induced effect on peak ASR magnitude. A mixed-model ANOVA conducted on peak ASR latency revealed a significant effect for PPI trial with a prominent linear trend, [F(1, 12)=43.34, p<0.001], but no effect for treatment [F(1, 12)=2.20, p=0.164) or treatment×PPI trial interaction [F(3, 36)<1.0]. FIG. 36a illustrates the peak ASR latency across ISI (0-4000 msec). Even though no treatment effect was noted, a slowing in response latency (4 msec, ˜15%) across all PPI trials (08-120 msec) was noted (FIG. 36b).

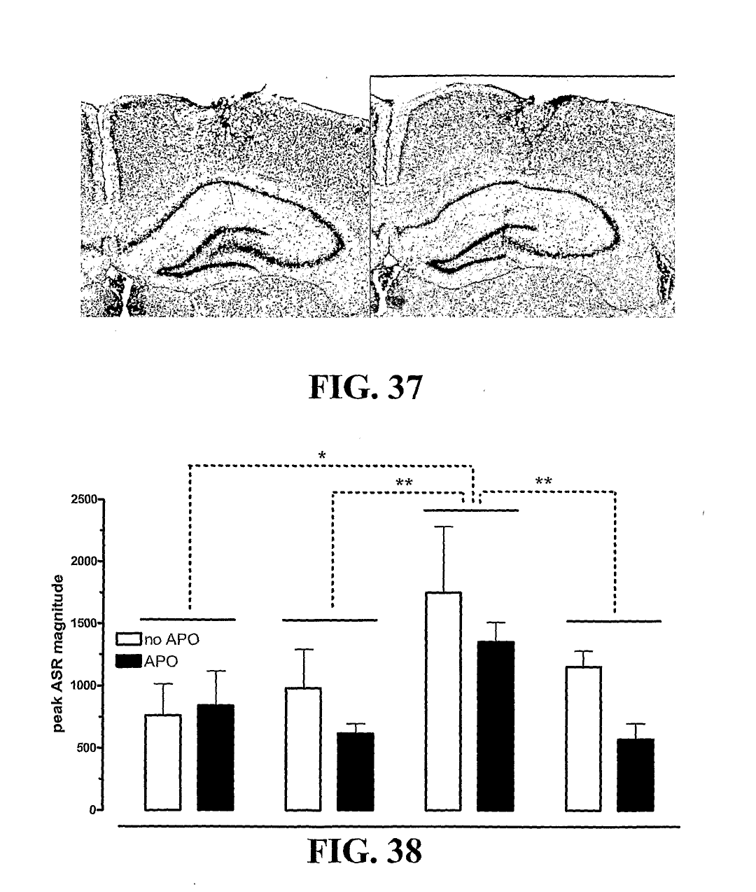

Histology: Analysis of Nissel-stained sections through the hippocampus confirmed placement of injection sites into the hippocampal dentate region. Saline-injected animals displayed little pathology, whereas damage to the CA3 and dentate region was noted in Tat-injected animals (FIG. 37).

### Example 10

### Intracerebral Hippocampal gp120 Injections: The Role of Dopaminergic Alterations in Prepulse Inhibition in Adult Rats

### Methods

Animals: Sprague-Dawley pregnant dams (n=8) were ordered from Harlan Laboratories, Inc. (Indianapolis, Ind.) and delivered to the vivarium before embryonic day seven. Dams were housed singly with food (Pro-Lab Rat, Mouse Hamster Chow #3000, NIH diet #31) and water available ad libitum. The day, pups were found in the cage was designated as postnatal day (P) 0. On PI, lifters were culled to 10 offspring of equal sexes, if possible. No more than one female and one male per litter were assigned to a single condition. The animal facility was maintained at 21±2° C., 50%±10% relative humidity and had a 12-hour light: 12-hour dark cycle with lights on at 0700 h (EST). The animals were maintained according to the National Institute of Health (NIH) guidelines in AAALAC-accredited facilities. The experimental protocol was approved by the Institutional Animal Care and Use Committee (IACUC) at the University of South Carolina, Columbia.

Gp120: Purified gp120 LAV (T-tropic) envelope protein was purchased from Protein Sciences Corp. (Meriden, Conn.] with a concentration of 100 μg at 1.0 ml. The recombinant HIV-1 gp120 was produced in insect cells and was >90% pure. Gp120 was stored at −20° C. until used for testing. Solutions of gp120 were prepared in order to get the following three doses: 1.29 ng/kg, 12.9 ng/kg, and 129 ng/kg. These three doses were used in this study to determine the gp 120-dose response function.

Surgery: The procedure was perfected in the laboratory at the University of South Carolina, Columbia. Standard stereotaxic surgery techniques were used for protein injections. The instrument used for stereotaxic surgery was obtained from KOPF® instruments. The syringe was obtained from Hamilton Co., Nevada, USA (Microliter # 701 R N, 10 μl). Individual pups were gently removed from the dam and immersed in ice anesthesia before being placed in a modified stereotaxic holder for surgery. Rubber head bars held the skull in place while bilateral microinjections of gp120 were made directly into hippocampus using the stereotaxic coordinates for injections according to a standard rat stereotaxic atlas. The set of coordinates used for the hippocampus were: right hemisphere −0.3 mm anterior to the bregma, 0.7 mm lateral to bregma, −3.0 mm dorsal from dura; left hemisphere −0.3 mm anterior to the bregma, −0.7 mm medial to bregma, −3.0 mm dorsal from dura. The 111 injection volume was released over one minute after a one-minute resting period that allowed the tissue to return to its original conformation. The needle was withdrawn over 2 minutes to prevent reflux. After the two injections, the piercings in the skin of the head were closed with Dermabond and the pups warmed under a heat lamp (35° C.) before being returned to the dam, where they were closely observed for indications of rejection. No pups were rejected or abused by the dam.

Experimental Design: Animals were group housed and randomly assigned to one of four gp120 treatment conditions that received bilateral hippocampal injections at P1 of either 1 μl volume saline as a buffer (n=5), 1.29 ng/kg gp120 as the low dose group (n=6), 12.9 ng/kg gp120 as the medium dose group (n=5), or 129 ng/kg gp120 as the high dose group (n=8). In each of the groups males and female rats were included. With 10 month of age animals were tested for sensory gating by assessing PPI of the ASR. One month later, in order to test the role of dopamine D1/D2 agonist, the same adult rats received apomorphine (APO) and were again tested for PPI.

Apparatus: The startle chamber (SR-Lab Startle Reflex System, San Diego Instruments, Inc.) was enclosed in an 81×81×116-cm isolation cabinet (Industrial Acoustic Company, INC., Bronx, N.Y.). Each animal was tested individually in the dark with a high-frequency loudspeaker, mounted inside the chamber 31 cm above the Plexiglas cylinder that produced a background white noise (70 dB(A)). The startle chamber consisted of a Plexiglas cylinder 8.75 cm in interval diameter resting on a 12.5×20-cm Plexiglas stand. The animal's response to the stimulus produced deflection of the Plexiglas cylinder, which is converted into analog signals by a piezoelectric accelerometer. Acoustic stimulus intensities and response sensitivities were calibrated using a SR-LAB Startle Calibration System. Sound levels were measured and calibrated with a sound level meter (Quest electronics: Oconomowoc, Wis.) with microphone placed inside the Plexiglas cylinder. The signals were then digitized (12 bit A to D) and saved to a hard disk.

Testing Procedures: All rats were tested approximately 20 min. Animals were first exposed to a 5-minute acclimation period of 70 dB(A) background, followed by six single stimuli, and 36 PPI trials with 0, 8, 40, 80, 120, and 4000 msec interstimulus interval (ISI), assigned by Latin-square design. The six single stimuli were defined as adaptation trials and the PPI trials 0 and 4000 msec were the control trials in order to control for PPI. The prepulse stimulus intensity was 85 dB(A). For PPI the dependent measures analyzed were peak ASR magnitude, peak ASR latency, and percent PPI. Percent PPI indicates the percent of inhibition in startle magnitude at a prepulse of 100 msec ISI relative to pulse only trials (0 msec ISI). PPI for ISI 100 msec was calculated using the average of PPI trials 80 and 120 msec ISIs. Percent PPI was computed according to the following formula: % PPI=[(0 msec ISI trials −100 msec ISI trials)/0 msec ISI trials]*100.

Data analysis: All data were analyzed using analysis of variance (ANOVA) techniques (SPSS, 2003; SYSTAT, 2003; Winer, 1971). Two-way mixed-factor ANOVAs, with gp120 dose as a between-subject factor (buffer, 1.29-, 12.9-, and 129 ng/kg) and drug condition as a within-subjects factor (no apomorphine vs. apomorphine) were performed on peak ASR magnitude and peak ASR latency in the adaptation trials, control trials and on percent PPI (through 100 msec). Three-way mixed-factor ANOVAs, with PPI trials added as a within-subjects factor, were performed on peak ASR magnitude and peak ASR latency across PPI trials (8-120 msec). The Greenhouse-Geisser df correction factor and contrast analyses were employed for violations of sphericity in repeated measures (Winer, 1971). Separate trend analyses for simple effects of PPI trials were employed to evaluate the nature of the PPI trial-dependent effects. This statistic also describes the shape of a trend by determining its significance (e.g., linear, quadratic, etc. trends). Trend analyses for simple effects of PPI trials were further analyzed when appropriate. Post hoc multiple comparison tests using the Bonferroni correction was used to determine specific gp 120 dose and/or drug effects. In addition, ISI in which the peak occurred was recorded across all PPI trials and categorized into two categories. Category one included ISIs 08 and 40, and category two included ISIs 80 and 120. The ISI data are categorical in nature; thus, the Fisher's exact test was applied. An alpha level of p<0.05 was considered significant for all statistical tests used.

### Results

ASR test: A 4 (gp120 dose)×2 (drug) mixed-model ANOVA on peak ASR magnitude in the adaptation trials revealed a significant gp120 dose effect [F(3, 20)=6.33, p=0.003], and a significant drug effect [F(1, 20)=7.08, p=0.015], but no gp120 dose×drug interaction [F(3, 20)<1.0]. For the gp120 dose effect, post hoc multiple comparison tests revealed significant differences between medium dose gp120-treated animals and all the other gp120 dose treatments [buffer: p=0.033; low dose: p=0.007; high dose: p=0.006]. Further, the dopamine D1/D2 receptor agonist APO attenuated the baseline of peak magnitude of the ASR, but did not significantly alter the gp120 neurotoxicity (see FIG. 38). A4 (gp120 dose)×2 (drug) mixed-model ANOVA on peak ASR latency revealed no gp120 dose or drug effects or interaction.

Prepulse Inhibition Test (PPI): Control Trials (0, 4000 msec ISI combined). A 4 (gp120 dose)×2 (drug) mixed-model ANOVA on peak ASR magnitude revealed a marginal gp120 dose effect [F(3, 20)=2.94, p=0.058], a marginal drug effect [F(1, 20)=3.54, p=0.075], but no gp120 dose×drug interaction [F(3, 20)<1.0], suggesting equivalent rates on the peak magnitude of the ASR. A 4 (gp120 dose)×2 (group) mixed-model ANOVA on peak ASR latency revealed no significant effects.

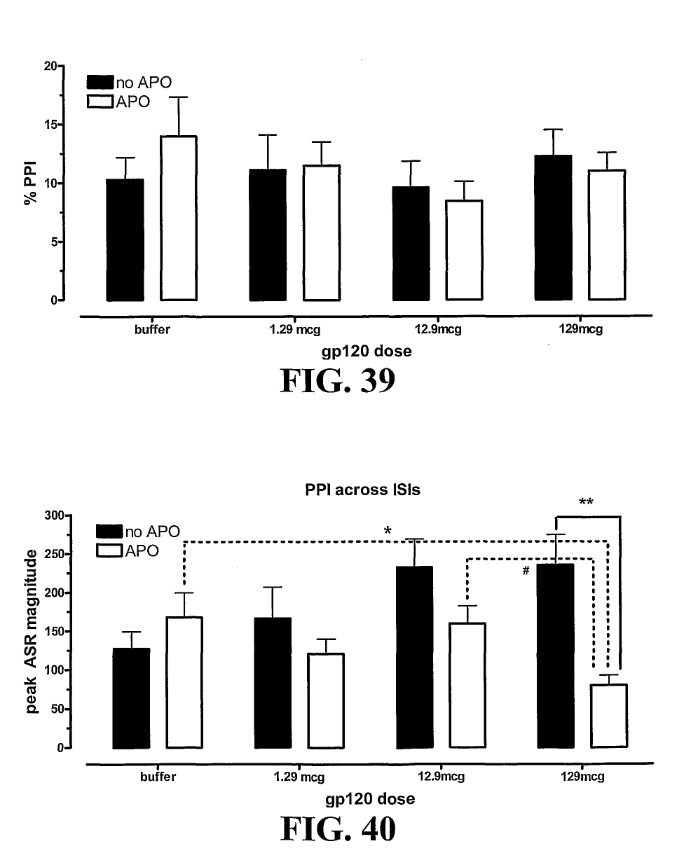

PPI trial [100 msec ISI]. A 4 (gp120 dose)×2 (drug) mixed-model ANOVA conducted on percent inhibition of the startle magnitude for ISI 100 msec revealed no effects [gp120 dose effect: F(3, 20)<1.0, drug effect: F(1, 20)<1.0, and gp120 dose×drug interaction: F(3, 20)<1.0], indicating no significant effect on inhibition response for gp120 treatment and drug condition when measured by percent PPI (see FIG. 39).

PPI trials: [8-120 msec ISIs] A 4 (gp120 dose)×2 (drug)×4 (PPI trials) mixed-model ANOVA conducted on peak ASR magnitude [PPI trial: Mauchly's W=0.026, p<0.001; drug×PPI trial: Mauchly's W=0.096, p<0.001] revealed no overall gp120 dose effect [F(3, 20)=1.11, p=0.370], but a significant drug effect [F(1, 20)=6.92, p=0.016]. The two-way gp120×drug was significant [F(3, 20)=3.62, p=0.031], indicating that the dopamine D1/D2 receptor agonist APO significantly altered the gp120 dose response. Simple main effects for drug revealed only a significant effect in the high gp 120 dose-treated animals [F(1, 7)=15.02, p=0.007]. Simple main effects for gp120 treatment revealed no gp120 dose effect when adult rats were not treated with APO [F(3, 20)=1.87, p=0.168]. A significant gp120-dose dependent effect however, was noted one month later when the dopamine D1/D2 receptor agonist APO was administered [F(3, 20)=4.19, p=0.019], with a decrease in peak magnitude as gp120 doses increased. Post hoc multiple comparison tests using Bonferroni as a correction factor revealed a significant effect between buffer- and high dose-treated animals (p=0.035) and a marginal effect between medium- and high dose-treated rats (p=0.069), thus suggesting that APO treated rats were more sensitive to gp120 neurotoxicity, as indexed by dose-dependent PPI-disruptive effects (see FIG. 40). Further, an overall PPI trial effect was noted [F(3, 60)=35.77, p_{GG}<0.001] with a prominent linear trend [F(1, 20)=43.12, p<0.001], but no gp120 dose×PPI trial interaction [F(9, 60)<1.0], only a marginal PPI trial×drug interaction [F(3, 60) 2.81, p_{GG}=0.096] with a significant linear trend [F(3, 20)=4.58, p=0.045], and no significant three-way gp120 dose×drug×PPI trial interaction [F(9, 60)=1.47, p_{GG}=0.240]. In addition, Fisher Exact probability tests on the ISI in which the peak occurred were conducted comparing drug effects separate for each gp120 dose (i.e. buffer-treated adults vs. buffer-treated APO). Further, Fisher Exact probability tests on the ISI in which the peak occurred were conducted comparing buffer treated adults with all gp120 treated animals within each drug treatment. No peak shift was noted in any of the tests, indicating no alteration in the maximal inhibition response across ISIs by gp120 and APO.

A 4 (gp120 dose)×2 (drug)×4 (PPI trials) mixed-model ANOVA conducted on peak ASR latency [Mauchly's test not significant] revealed only a significant PPI trial effect [F(3, 60)=45.12, p<0.001] with a prominent linear component [F(1, 20)=96.50, p<0.001], indicating a linear slowing in response with an increase in ISIs between the prepulse and the startle stimuli. No gp 120 treatment or/and drug effect/interactions were noted, suggesting that gp120 and/or APO did not have an impact on latency response.

Thus, dopamine D₁/D₂ agonists can enhance neuronal response to gp120 neurotoxicity that result in PPI-disruptive effects, indicating attentional deficits.

### Example 11

### Contribution of Dopamine Agonist Interaction with Primary Binding Sites in Cocaine+Tat Induced Oxidative Cell Damage

Determined herein is whether the enhancement of neurotoxic effects of Tat is mediated by dopmine-induced changes in functioning of DA, SE, or NE neurotransmitter systems and the extent of the involvement of particular monoamines in the synergistic toxicity of Tat and estrogen protection in primary rat cell cultures is evaluated.

TABLE 2
Experimental Design

| | | | |
|---|---|---|---|
| Control | DAT | SERT | NET |

Control
HIV
HIV + D-1 drug
HIV + Estrogen(E)
HIV + D-1 + E

Experimental Design: All treatments include at least 8 sister cultures and 3 replications are performed to confirm the reproducibility of the observed effects. The drugs used in this experiment are detailed in Table 2. Initially the range of non-neurotoxic concentrations is established for each compound in order to determine an optimal concentration of each type of selective monoamine transporter inhibitor that is used for cell culture treatment. Tat is used at 50 nM concentration that was shown to produce moderate neurotoxicity in primary rat hippocampal cell cultures. At 2 hours and 48 hours after treatment the following is determined:

a) intracellular ROS production using free radical-sensitive fluorescent probe dichlorofluorescein diacetate (H₂DCFDA),

b) mitochondrial membrane potential using JC-1 fluorescent mitochondrial probe,

c) biomarkers of protein oxidative damage using specific immunostaining techniques (protein carbonyls, nitrotirosines, 4-hydroxy-2-nonenal (HNE)-modified proteins),

d) cell viability using a Live/Dead viability/cytotoxicity kit (TUNEL assay is also be used to detect apoptotic cells in the experimental groups of cell cultures), and

e) functional uptake of NE, DA or 5-HT into neurons following each of the treatments.

Methods

ROS indicator: H₂DCFDA is obtained from Molecular Probes (Eugene, Oreg.). H₂DCFDA passively diffuses into cells where the acetates are cleaved by intracellular esterases to form H₂DCF and thereby trapping the product within the cell. Loading of cultured cells with the probe (10 M final concentration) is performed for 60 min at 37° C. in growth medium. Formation of the oxidation product 2′,7′-dichlorofluorescein (DCF) will produce bright green fluorescence in the cell.

Mitochondrial membrane potential probe, JC-1, also is obtained from Molecular Probes. The potential-sensitive color shift of this cationic dye is due to concentration-dependent formation of red fluorescent J-aggregates. For determination of mitochondrial function, cells are loaded with JC-1 (10 μM final concentration) for 60 min at 37° C. in growth medium.

Immunostaining of cell cultures for biomarkers of oxidative damage is performed using the basic protocol for protein carbonyl immunostaining of cultured rat hippocampal neurons that was previously developed by us (Aksenova et al., 1999). This protocol is modified to allow measurements of different types of oxidative modifications of neuronal proteins using a microplate reader. Following the fixation cell cultures prepared in 96-well microplates are stained with fluorescent total protein stain, SYPRO (Molecular Probes), which does not interfere with immunostaining techniques and then processed for determination of reactive carbonyl, nitrotirosines, and HNE groups in cell proteins by specific antibodies. Source of the antibodies: Rabbit polyclonal antibodies which recognize protein carbonyls following their derivatization with 2,4-dinitro-phenylhydrazine (DNPH) (Levine et al., 1990, 1994) are obtained from Intergen (Purchase, N.Y.). Working dilution is 1:100. Mouse monoclonal anti-nitrotirosine antibodies (Upstate Biotechnology, Lake Placid, N.Y.) and are used at working dilution 1:200, as recommended by the provider. Rabbit polyclonal anti-HNE antibodies (Alpha Diagnostic International, San Antonio, Tex.) are used for immunocytochemistry at 1:500 dilution.

Neuronal viability is determined using a standard protocol for calcein/ethidium bromide staining of live/dead cultured neurons (Molecular Probes) adapted for a microplate reader. In accordance with the manufacturer's protocol, neurons are exposed to cell-permeant calcein AM (2 μM) which is hydrolyzed by intracellular esterases, and to ethidium homodimer-1 (4 μM) which binds to nucleic acids. The cleavage product of calcein AM, calcein, produces a green fluorescence when exposed to 494-nm light and is used to identify live cells. Bound ethidium homodimer-1 produces a red fluorescence when exposed to 528-nm light allowing the identification of dead cells. To detect cells with DNA damage, the peroxidase in situ TUNEL staining kit (Roche Molecular Biochemicals, Mannheim, Germany) is used according to the manufacturer's instructions.

All quantitative analyses of optical densities and fluorescence are carried out using a Bio-Tek Synergy HT microplate reader (Bio-Tek Instruments, Inc., Winooski, Vt.). The use of microplate-based protocols allows simultaneous analysis of big sets of samples and ensures non-biased quantitative analysis of the effects of cell culture treatments. Results of high output microplate-based analyses are confirmed by laser confocal imaging of control and experimental neuronal cell cultures. For laser confocal imaging analyses, primary rat hippocampal cell cultures are grown on specifically designed glass-bottom culture dishes and treated the same way as cultures prepared in 96-well plates. a Nikon C-1 Laser Confocal Microscopy system, which supports simultaneous three channel confocal fluorescence detection while simultaneously capturing scanned differential interference contrast (DIC) images (Nikon Instruments Inc. Melville, N.Y.) is used to obtain high resolution confocal images. Minor adjustments of fluorescent staining conditions compared to microplate based protocols are used to optimize them for laser confocal imaging.

Functional uptake of monoamines in vitro. At day 12, Tat and cocaine are added to the cultures. After 24 hours, medium is removed and replaced by the uptake buffer (25 C; 25 mM HEPES, 120 mM NaCl, 5 mM KCl, 2.5 mM CaCl₂, 1.2 mM MgSO₄; 1 μM pargyline, 2 mg/ml glucose, 0.2 mg/ml ascorbic acid). The selective uptake inhibitors are added and preincubated (time to be determined in preliminary studies). After pre-incubation, [3H]dopamine is be added to a final concentration of 20 nM to initiate uptake for the appropriate time/temperature to steady state levels. Unlabeled dopamine is added as needed for determination of Km and Vmax. The assay is terminated by aspiration of supernatant and washed 2× with ice cold PBS. Scintillation fluid is then added to the microbeta plates and radioactivity remaining in the cells determined. Optimal time course and temperatures will be determined for each drug. To rule out metabolism of [³H]dopamine, HPLC-EC is used to verify that the intracellular signal is dopamine in a limited number of samples. Non-linear curve fitting is used to determine Km and Vmax values.

Neuronal cell cultures: The choice of hippocampal neuronal cell culture is based evidence for significant sensitivity of this region of the brain to HIV-associated neuropathology (Petito et al., 2001) and the presence of DAT, SERT and NET in these fetal cell cultures (DAT present in approximately 40% of cells).

Primary rat fetal hippocampal neuronal cell cultures are prepared from 18-day-old Sprague-Dawley rat fetuses (Aksenov et al., 1998; Aksenova et al., 1999; Aksenova et al., 2005). In brief, rat fetal hippocampal neurons are plated at the initial density of 140-160 cells/mm² and cultures are maintained in an optimized serum-free medium (B-27 Neurobasal medium, GIBCO Life Technologies) (Brewer et al, 1993). This medium supports excellent survival of neurons even at low density and prevents glial proliferation (Puttfarcken et al., 1996; Zang et al., 1996). Under these culture conditions, the cells reach fully mature state by 12-14 days in culture. At this time, cultured neurons exhibit maximal intensity of oxidative metabolism but no sign of damage by free radical by-products (Aksenova et al., 1999). Thus, all experiments are conducted at 12 days in culture. The day before the experiments, B-27-supplemented Neurobasal medium is replaced with Neurobasal medium without antioxidants (-AO supplement replaced B-27 supplement). Fetal rat hippocampal neurons are cultured in either 96-well plates (for high output analyses using microplate reader), in glass-bottom plates (for laser confocal imaging and immunoanalyses), in 4 cm plastic dishes (for total RNA extraction), or in microbeta plates (uptake assays).

Recombinant proteins: Both 1-72 (first exon) and 1-86 (full length) variants of recombinant Tat are toxic to cultured neurons (Bonavia et al, 2001; Kruman et al, 1998) and the region spanning from amino acid residue 31 to 61 is essential for the neurotoxicity of the protein (Marbrouk et al., 1991; Sabatier et al., 1991; Philippon et al., 1994; Nath et al., 1996). Tat 1-72 is not internalized by neurons (Ma and Nath, 1997) and the membrane-triggered events play key role in Tat neurotoxicity (Sabatier et al., 1991; Cheng et al., 1998). Recombinant Tat 1-72 (first exon) is therefore used herein in order to separate the events caused by Tat interaction with neuronal membranes from various effects that full-length Tat can trigger after internalization. When indicated, the biologically inactive analog of Tat Δ31-61 is used to control for the specificity of Tat effects. Although nanomolar concentrations of Tat can induce cell death in neuronal cell cultures (Bonavia et al, 2001), the dose range of recombinant Tat 1-72 or 1-86 proteins used for cell culture treatments varies considerably from 10 to 1000 nM (Bonavia et al, 2001; Kruman et al, 1998; Perez et al, 2001; Turchan et al, 2001). Thus, a relatively low 50 nM dose of Tat is used herein for treatment of neuronal cell cultures.

Data Analyses: For group analyses, analysis of variance (ANOVA) techniques are used (BMDP, 1993) and planned comparisons are used to determine specific treatment effects. Regression analysis is used to determine dose response relationships (Winer, 1971). An α level of p<0.05 is used for all analyses. GraphPad Prizm is used to analyze uptake experiments via non-linear curve fitting for estimates of Km and Vmax.

### Results

As determined by the methods provided herein, the enhancement of oxidative stress-dependent neurotoxicity induced by HIV-1/Tat is mediated via the DA system. The relative role and specificity of different monoamine transmission systems in the enhancement of Tat-induced oxidative stress and neurodegeneration by cocaine is determined by the selective pharmacological modulation of the activity of DA-1 receptors expressed in rat hippocampal cell cultures. Moreover, whether the inhibition of neurotransmitter reuptake is critical in producing the oxidative stress generated by HIV-/Tat is determined by the herein provided methods. These findings suggest pharmacological strategies, such as estrogens, which, by maintaining neurotransmitter reuptake, can prevent oxidative stress and cell loss.

### Example 12

### Estrogen Receptor Subtype Stimulation in Neuroprotection Against Tat+Cocaine

Determined herein is how estrogen neuroprotection of HIV-1/Tat neurotoxicity is mediated. 17-β-estradiol has a specific, estrogen receptor mediated, neuroprotective action against Tat+cocaine induced neurodegeneraton (Turchan et al., 2001; Kendall et al., submitted). Moreover, estrogen was capable of protecting against the damage to DAT produced by Tat exposure. First, determined is whether protection by 17-β-estradiol is specifically mediated via the alpha or beta receptor subtypes of the estrogen receptor. A comprehensive series of studies with series of selective estrogen receptor antagonists and agonists is provided to determine which estrogen receptor subtype is responsible for the neuroprotective effects and whether a selective estrogen receptor modulator, raloxifene, is an effective neuroprotectant. In women, estradiol is present in pM quantities, so doses within this physiological range are employed. Also determined is the specificity of estrogen's neuroprotective actions relative to DAT in reducing oxidative stress indicators as determined herein.

TABLE 3
Experimental Design.

| | | | | |
|---|---|---|---|---|
| | | D-1 | | Tat + |
| Control | Tat | drug | Tat + D-1 | GBR12909 |

Control
E2
OHT
Raloxifene
ICI182,780
PPT
DPN
PPT + OHT
DPN + OHT
PPT + DPN

Experimental Design: Whether cultures express significant amounts of estrogen receptor subtypes is confirmed by immunocytochemistry using antisera to estrogen receptors and by western blot analysis. Hippocampal cell cultures express both alpha- and beta-estrogen receptors in relatively high amounts (Su et al., 2001), and estrogen receptors are present in the fetal cultures (Turchan et al, 2001). Co-localization studies with ER and DAT antibodies are performed using confocal microscopy.

The specific estrogen receptor agonists and antagonists used herein are detailed in Table 4.

TABLE 4
Selected estrogenic agonists/antagonists

| Compound | Dose (nM) | Mechanism of Action |
|---|---|---|
| 17-β-estradiol | 0.001-10 | Biologically active at the estrogen receptors |
| 4-OH-Tamoxifen (OHT) | 0.001-0.1 | Tamoxifin is a non-steroidal anti-estrogen |
| Raloxifene | 0.001-0.1 | Raloxifene is a mixed agonist-antagonist SERM |
| 17-β-estradiol + ICI 182,780 | 0.001-1 | ICI 182,780 is a highly selective pure steroidal |
| | | antagonist at estrogen receptors |
| Propyl-pyrazole-triol (PPT) | 0.001-10 | PPT is a selective estrogen receptor-alpha |
| | | subtype agonist |
| Diarylpropionitrile (DPN) | 0.001-10 | DPN is a selective estrogen receptor-beta |
| | | subtype agonist |
| PPT + tamoxifen | 0.001-10 | Tamoxifen will block any selective PPT effects |
| DPN + tamoxifen | 0.1-100 | Tamoxifen will block any selective DPN effects |
| PPT + DPN | 0.1-100 | Combined alpha and beta effects, similar to 17- |
| | | β-estradiol |

At 2 hours and 48 hours after treatment the following is determined:

a) intracellular ROS production using free radical-sensitive fluorescent probe dichlorofluorescein diacetate (H₂DCFDA),

b) mitochondrial membrane potential using JC-1 fluorescent mitochondrial probe,

c) biomarkers of protein oxidative damage using specific immunostaining techniques (protein carbonyls, nitrotirosines, 4-hydroxy-2-nonenal (HNE)-modified proteins),

d) cell viability using a Live/Dead viability/cytotoxicity kit (TUNEL assay is also used to detect apoptotic cells in the experimental groups of cell cultures), and

e) functional uptake of DA into neurons following each of the treatments.

### Results

First, whether 17-β-estradiol is neuroprotective against Tat neurotoxicity is confirmed and that this is a receptor dependent phenomenon. Next, using PPT (ER-alpha) and DPN (ER-beta) whether this neuroprotection is mediated by either subtype of estrogen receptor is determined, thereby identifying a potential pharmacological target for neuroprotective strategies. Finally, whether this ER subtype selective compound can protect against the combined toxicity of GBR-12909 is determined. Intracellular oxidative stress is used herein as a biomarker, as oxidative stress is causal to neurotoxicity (Askenova et al., 2005) and modulation of oxidative stress is key to Tat+cocaine toxicity.

### Example 13

### The Role of Estrogen in Modulating Dopaminergic System Function Following HIV-Infection

Determined herein is the role of estrogen in the expression of Tat neurotoxicity on DAT via in vivo microdialysis in awake, freely moving, animals. The time course and dose-response of behavioral activation and sensitization to IV cocaine is compared in female rats lacking gonadal hormones, as well as those with specific hormone replacement (estrogen) following Tat injection. In vivo microdialysis has been used extensively to determine the concentrations of catecholamines/indolamines in the rodent brain (Benveniste & Huttemeier, 1990; Robinson & Whishaw, 1988). The ability of Tat to modulate DAT and dopamine receptors in response to chronic cocaine administration in the nucleus accumbens is examined using receptor autoradiographic techniques (Booze & Wallace, 1995; Wallace & Booze, 1995a; 1995b). These experiments assess the physiological relevance of estrogen protection on DAT functioning in vivo.

Experimental Design: These data provide a direct assessment of the effects of 17-β-estradiol (E2) on and Tat toxicity in adult female animals. In this experiment, ovariectomized female animals (OVX), will be compared to ovariectomized animals with estrogen replacement (OVX+E). Animals are intracerebrally injected with Tat into the nucleus accumbens. First, in Acute Tat experiments, Tat injection co-occurs during the microdialysis session. Thus, the time course of tat-DA interactions can be monitored and the acute cocaine response determined. Second, in Prior Tat experiments, brain tissue is processed for determination of dopamine markers using receptor autoradiography. Trunk blood is collected at sacrifice for RIA analysis and confirmation of estrogen levels in all animals and the levels of dopamine markers in the nucleus accumbens determined. The experimental design groups are outlined in Table 5.

TABLE 5
Microdialysis session treatments

| | Acute | | | |
|---|---|---|---|---|
| | Saline - | Acute Tat - | Prior Saline - | Prior Tat - |
| | concurrent | concurrent | 24 hours | 24 hours |
| OVX | | | | |
| OVX + E | | | | |
| OVX + DA-1 | | | | |
| OVX + E + DA-1 | | | | |
| OVX | | | | |
| OVX + E | | | | |
| OVX + DA14 | | | | |
| OVX + E + DA14 | | | | ns = 10 |

Estrogen Treatment (OVXvs. OVX+E): Ovariectomized adult female Sprague-Dawley rats with implanted IV catheters (Mactutus et al., 1994; Wallace et al., 1996; Harrod et al., submitted) are obtained from Harlan Laboratories. Following a two week recovery period to allow full depression of endogenous steroid levels and recovery from shipping, rats are randomly assigned into 2 steroid treatments groups according to the type of subcutaneous implant. Specifically, one group of OVX animals receive crystalline 17-β-estradiol in 5 mm Silastic tubing (OVX+E) and a second group of OVX animals receive sham implants (cholesterol in Silastic tubing). The E implants provide stable levels of estrogen for several weeks (Huang et al., 2004; Ratka & Simpkins, 1990) which would normally be experienced by female rats on the day of proestrus (50-60 pg/ml).

DA agonist Treatment (DA1 vs. DA14): One week following pellet surgery, animals receive either 14 days saline (DA1) or 14 days of IV cocaine (DA14) with locomotor activity testing on days 1 and 14. Animals receive 1 injection/day of either saline or one DA agonist (e.g., SCH23390, Research Biochemicals) for days 2-13 in their home cages. On the first and 14th day immediately after injection, animals are placed in the BAS microdialysis chamber. DA drugs are administered and behavioral observations in the BAS chamber are conducted. Behavioral sensitization to repeated cocaine treatment are confirmed. Prior Tat treatment/guide cannula placement occurs on DA/SAL day 13, with behavioral testing and microdialysis with drug administration occurring on day 14.

Tat Treatment (Acute Tat vs. Prior Tat): In order to define the time course of Tat effects on accumbens DA efflux, Tat 1-72 protein (15 μg in 1.0 μl; infused over a time period of 5 min) or its vehicle (saline) is infused into the nucleus accumbens core either 24 hr prior to microdialysis (Prior Tat) or concurrently during the microdialysis session (Acute Tat).

In vivo microdialysis procedures: Guide cannulae are placed on the day prior to microdialysis. Animals are anesthetized (ketamine/xylazine) and placed in a stereotaxic apparatus. A combination intracranial infusion cannula/microdialysis guide cannula (Bioanalytical Systems, Inc., W. Lafayette, Ind.) are directed to the nucleus accumbens core (NAC) according to the following coordinates, relative to Bregma: AP +1.6 mm, L +1.5 mm, DV −6.0 mm. Tat is injected through the guide cannula.

On the day of microdialysis, awake, freely-moving, animals are placed in the dialysis chambers and the combination infusion cannula/microdialysis probe is inserted into the guide cannula. The semipermeable membrane portion of the microdialysis probe extends 2 mm beyond the tip of the guide cannula and is perfused (2.0 μl/min) with an artificial cerebrospinal fluid (aCSF) as composed of the following (in mM); NaCl 126.5, NaHCO₃ 27.5, KCl 2.4, Na₂SO₄ 0.5, KH₂PO₄ 1.1, CaCl₂ 1.1, MgCl₂ 0.8 and D-glucose 1.0. Dialysate collection begins 3 hr after probe insertion, a time point at which basal accumbens DA efflux is stable and greater than 90% TTX-sensitive. Dialysates are collected in 15 min fractions into vials containing 5.0 μl of 0.1N perchloric acid to minimize spontaneous oxidation of DA prior to chromatographic analysis. Four baseline dialysate fractions are collected. Half of the animals then receive intra-accumbens infusions of TAT or saline. Behavioral activity is monitored during the microdialysis session in the BAS chamber. Dialysate collection continues for an additional 2 hr, prior to IV delivery of cocaine or saline. Dialysis continues for an additional 2 hr and the probes are then removed.

Neurochemical and statistical analysis: DA/5-HT and metabolite levels in dialysates are determined by HPLC with electrochemical detection (Lunte & Lunte, 1996; Imperato & Di Chiara, 1984). Briefly, catecholamines are separated from other electroactive dialysate components on a 5 μm C18 analytical column (BAS) using a mobile phase (0.75 ml/min) containing 0.1 M citrate, 1.2 mM HSA, 75 mM Na₂HPO₄, 0.1 mM EDTA, and 14% methanol (final pH 4.3). After separation, the chromatographic eluate is directed to a radial-flow glassy carbon working electrode, where DA is oxidized by an applied potential of +0.3V. The current corresponding to this reaction is plotted as a peak using “Chromgraph” software (BAS), and values are determined in reference to a daily 4-point standard curve. Raw efflux data (fmol DA/μl dialysate) are analyzed by ANOVA (SPSS for PC). The source of significant (p<0.05) main effects or interactions will be determined by Bonferroni comparisons.

Steroid levels are determined at sacrifice by collecting trunk blood and determining estradiol levels by radioimmunoassay (RIA) (Huang et al., 2004) (Diagnostic Products Corporation, Los Angeles, Calif.).

Dopamine transporter and receptor subtypes are analyzed in the striatum and nucleus accumbens using quantitative receptor autoradiography (Booze & Wallace, 1995; Wallace & Booze, 1995a). Animals are sacrificed, the brains removed and cryostat-sectioned (20 μm). Microdialysis probe placement in the nucleus accumbens core is verified using Nissl stained sections through the nucleus accumbens. In brief, the binding parameters for quantitative receptor autoradiography of the D₁, D₂ and D₃ receptors and Dopamine transporters are outlined in Table 6.

TABLE 6
Summary of conditions for dopamine receptor autoradiography

| Receptor | Ligand | Pre-wash | Incubation | Post-wash | Nonspecific |
|---|---|---|---|---|---|
| D₁* | 1.0 nM | 15 min | 30 min @22° C. | 2 × 10 min | 10⁻⁶ M |
| | [³H]-SCH 23390 | 50 mM Tris-HCL; | as prewash | 4° C., Tris-HCL | SCH 23390 |
| | | 5 mM KCl; 2 mM | with ligand | buffer | |
| | | CaCl₂; 1 mM | | | |
| | | MgCl₂; 120 mM NaCl | | | |
| D₂+ | 1.0 nM | 15 min | 60 min @22° C. | 2 × 10 min | 1.0⁻⁶ M |
| | [¹²⁵I]-Epidepride | 50 mM Tris-HCL; | as prewash | 4° C., Tris-HCL | (−)sulpiride |
| | | 5 mM KCl; 2 mM | with ligand + | buffer + ascorbic | |
| | | CaCl₂; 1 mM | idazoxan | acid | |
| | | MgCl₂; 120 mM NaCl | | | |
| D₃\hat{ } | 2.0 nM | 5 min | 60 min @22° C. | 2 × 10 min | 1.0⁻⁶ M |
| | [³H](+)7-OH- | 50 mM Tris-HCL; | as prewash | 4° C., Tris-HCL | Dopamine |
| | DPAT | 5 mM KCl; 2 mM | with ligand + | buffer + ascorbic | |
| | | CaCl₂; 2 mM MgCl₂; | carbetapentane + | acid | |
| | | 50 mM NaCl | Gpp(NH)p | | |
| DAT | 5.0 nM | 10 min | 90 min @22° C. | 2 × 2 min | 1.0⁻⁶ M |
| | [³H]Mazindol | 50 mM Tris-HCL; | as prewash | 4° C., Tris-HCL | Dopamine |
| | | 5 mM KCl; 2 mM | with ligand + | buffer | |
| | | CaCl₂; 10 mM | DMI | | |
| | | MgCl₂; 154 mM | | | |
| | | NaCl | | | |

*Conditions similar to McCabe et al., 1987.
+Conditions similar to Murray et al., 1992; Wallace & Booze, 1996.
\hat{ }Conditions similar to Staley & Mash, 1996; Wallace & Booze, 1995a; Wallace et al., 1999.

Following incubation, the tissue sections are washed twice for 10 min, dipped in dH₂O, dried under a stream of cool are and placed in a desiccator under vacuum overnight. The dried tissue sections are directly apposed to autoradiography film (Amersham) in light-tight autoradiography cassettes together with appropriate standards (Amersham). The films are developed after the optimal exposure time for each ligand has been determined. It is critical for quantitative densitometry to stay within the linear range of the autoradiographic film. Computer-assisted densitometry methods (MCID-4 System, Imaging Research) are used to quantify the number of dopamine receptors in the tissue sections (methods according to Booze et al., 1989; Booze & Wallace, 1995). Density readings are obtained from the dorsal striatum (matrix compartment), olfactory tubercle (including the islands of Calleja) and the core and shell subterritories of the nucleus accumbens. Core and shell subterritories are identified by heavy (shell) or light (core) acetylcholinesterase staining on adjacent tissue sections (Wallace & Booze, 1995a). The relative optical density data is expressed as fmol/mg wet weight for each brain region.

Data Analysis: All data (behavioral, microdialysis and autoradiography) are analyzed using analysis of variance (ANOVA) techniques (Winer, 1971; BMDP Statistical Software, 1993). For example, locomotor activity is analyzed by a repeated-measures ANOVA with between subject factors of estrogen treatment (3), Tat protein (4) and cocaine treatment (2) and within-subject factors of behavioral days (2). Data is transformed if they do not meet the assumptions of ANOVA. Any violation of compound symmetry for the repeated-measures factors is corrected by use of the Greenhouse-Geisser (1959) adjustment to df or avoided by use of orthogonal trend decomposition. Tests of simple main effects are used to analyze any significant interaction terms. An α level of p≦0.05 is used for all analyses.

### Results

In vivo microdialysis is conducted in awake, freely moving, animals. The levels of extracellular DA, 5-HT, DOPAC, HVA and 5-HIAA are determined using HPLC techniques. In those acute experiments in which Tat is perfused during the microdialysis experiment, an “on line” view of the ability of Tat to inhibit DAT under physiological conditions is obtained. Based on the methods provided herein, it is determined that DA levels increases with acute Tat infusion. The neurotoxic interactions with dopmine agonists and protection by estrogen are also examined. Damage to DAT and DA terminals are confirmed by quantitative autoradiographic analysis and standard neuropathologic analyses of the nucleus accumbens core region. Following the identification of the key processes involved, quantitative microdialysis (“no net flux analysis”) is performed for absolute measures of DA content in the nucleus accumbens (Parsons & Justice, 1994).

As determined by the herein provided methods, prior treatment with Tat damages DAT terminals and therefore, baseline levels of DA decrease long term (even though DA may be elevated in the nucleus accumbens after acute Tat treatment). Further, estrogen treatment maintains the functionality of DAT and the ability of DA agonists stimulate levels of extracellular DA and produce behavioral sensitization/locomotion.

### Example 14

### Intra-Accumbal Tat (1-72) Attenuates Intravenous Cocaine-Induced Behavior in Rats

### Materials and Methods

Animals: Sixty-four adult ovariectomized female, Sprague-Dawley rats (70 days old) were obtained from Harlan Laboratories, Inc. (Indianapolis, Ind.). All rats were surgically implanted with an Intracath IV catheter (22 ga, Becton/Dickinson General Medical Corp., Grand Prarie, Tex.), which was used as a SC, dorsally implanted port for chronic IV injections. The subcutaneous implantable access port for rats was developed and described by Mactutus et al. (1994). Ovariectomized rats were used because estrogen is protective against the neurotoxic effects of Tat (Wallace et al., 2006). Serial ovariectomy and indwelling jugular catheter surgeries occurred for all rats during the same procedure. Upon arrival at the animal care facilities, rats were placed in quarantine for 7 days, and then transferred to the colony. Animals were pair housed throughout the experiment and the catheters were flushed daily with 0.2 ml of heparinized (2.5%) saline. Rodent food (Pro-Lab Rat, Mouse Hamster Chow #3000) and water were provided ad lib. The colony was maintained at 21±2° C., 50%±10% relative humidity and a 12L: 12D cycle with lights on at 0700 h (EST).

Locomotor activity: Habituation: Rats in the vehicle-saline (VEH-SAL), Tat-saline (Tat-SAL), vehicle-cocaine (VEH-COC), and Tat-cocaine (TAT-COC) groups were habituated to the locomotor activity chambers for two 60-min sessions, one/day. Twenty four hours after the second habituation session, all rats were injected with IV saline and placed into the activity chambers for 30-min to measure baseline activity. The activity monitors were square (40×40 cm) open-field chambers (Hamilton-Kinder Inc., Poway, Calif.) that detected free movement of animals by infrared photocell interruptions. This equipment used an infrared photocell grid (32 emitter/detector pairs) to measure locomotor activity. The chambers were converted into round (˜40 cm diameter) compartments by adding clear Plexiglas inserts; photocell emitter/detector pairs were tuned by the manufacturer to handle the extra perspex width. Circular chambers increase the sensitivity to detect locomotor activity (Harrod et al., 2006). All activity monitors were located in an isolated room.

Intra-accumbal Tat microinjection: All rats received intra-accumbal Tat or vehicle (VEH; physiological saline) injections approximately 24 hours after the saline baseline activity session and then received subsequent IV injections of cocaine (3.0 mg/kg/injection) or saline according to a 2×2, protein×drug, design. The development of COC-induced sensitization is associated with structural changes in the core, but not the shell region of the N Acc (Li et al., 2004). Tat was therefore administered to the core region of the N Acc to determine if the sensitization process was disrupted. Rats were administered intra-accumbal Tat or VEH injections according to methods previously described (Bansal et al., 2000; Aksenov et al., 2003). Briefly, rats were anesthetized using a mixture of ketamine hydrochloride and xylazine by intraperatoneal (IP) injection (7.5 mg ketamine/100 gb.wt. 30 mg xylazine/100 gb.wt.). 15 μg/l of Tat was injected bilaterally, using a silanized Hamilton Syringe at the following coordinates: 1.8 mm anterior, ±1.6 mm lateral, and 7.0 mm ventral from bregma on the skull surface, over an interval of one minute, with a one minute tissue recovery period before withdrawal.

Injection of a ketamine/xylazine cocktail prior to cocaine can attenuate cocaine-induced behavioral sensitization when cocaine is delivered via the IP route of administration (Torres et al., 1994). These interactions relate to the temporal occurrence of the two drugs or repeated daily injections of ketamine (Rofael and Abdel-Rahman, 2002). In the present experiment, the ketamine/xylazine cocktail was administered before surgery, 24-h prior to the first cocaine injection. A single ketamine/xylazine injection preceding cocaine injection by 24-h should not affect the induction of behavioral sensitization.

IV cocaine-induced locomotor activity: Twenty-four hours after rats received bilateral intra-accumbal injections, IV cocaine or saline was administered. The animals were immediately placed into locomotor activity chambers following cocaine or saline. The animals' locomotor activity response to cocaine was assessed for 30 minutes on only two occasions: immediately after the first (Day 1) and last (Day 14) cocaine injections; however, rats were administered daily IV cocaine or saline injections in the home cage on days 2-13. This latter procedure is important to preclude the repeated pairing of cocaine injection and the testing environment that otherwise confounds the neural expression of sensitization with learning via classical conditioning (Anagnostaras and Robinson, 1996; Li et al., 2004).

Two automated measures of activity—total activity and total time in center (TTIC) of compartment—were investigated. Total activity represents all detectable movements that occur in the chamber. The TIC (seconds) spent in a circular region located in the center most portion of the compartment was the second dependent measure. Repeated IV cocaine injections produce an increase, or behavioral sensitization, of TIC in male and female rats (Wallace et al., 1996; Booze et al., 1999b). The increased TIC following acute and repeated IV injection is not likely due to an anxiolytic effect of the drug as COC has been shown to produce a dose dependent increase of anxiogenic behaviors in an elevated plus maze (DeVries & Pert, 1998; Paine et al., 2002). Rather, rats exhibit thigmotaxic behavior in open field environments and thus perform a number of locomotor and orofacial-related behaviors. The stimulant effects of drugs like COC and nicotine decrease the duration of time in the periphery by increasing locomotor activity, and hence time in the center portion of the open field (Wallace et al., 1996; Booze et al., 1999b; Harrod et al., 2004). Thus, COC-induced behavioral sensitization of locomotor activity is accompanied by a comparable sensitization of TIC. In order to determine the TIC, Hamilton-Kinder, Inc. software was used to impose a circular region (˜24% of total area) in the center of the compartment during the data reduction phase (i.e., following completion of the activity session) of the experiment, yielding the TIC data.

In addition to the automated monitoring, an observational time sampling procedure was employed. An observer, unaware of the treatment condition of the animal, observed and recorded the animal's behavior, using a well-established protocol (Fray, et al., 1980; Harrod et al., 2004; Harrod et al., 2005a; 2005b). 3.0 mg/kg/injection resulted in peak arterial plasma levels within one-minute of IV cocaine administration (Booze et al. 1997). Thus, five of the six observational time points occurred within the first 30 minutes of the session to insure that maximal amount of cocaine-induced locomotion would be observed. The current observational procedure represents an adaptation of Fray et al. (1980). Briefly, the present procedure excluded the sway and miscellaneous categories and added scan, headbob, yawn, and lying down. Another modification included observations at 1, 5, 10, 15, 30, and 60 min, whereas Fray et al. (1980) observed behaviors at 10, 20, 30, 40, 50, and 60 min. Table 7 describes the behaviors recorded during the sampling period. Testing occurred between 1500-1700 h under dim light conditions, in the absence of direct overhead lighting (<10 1×). All animals were sacrificed according to procedures used in Harrod et al., (2004). Brains were collected, frozen on dry ice, and stored at 80° C. Cryostat-cut sections (20 μm) were collected through the nucleus accumbens and Nissl-stained to verify injection sites.

TABLE 7
Descriptions of behaviors measured in the observational time sampling
procedure.

| Behavior | Description |
|---|---|
| Still | animal's body is completely still |
| Locomotion | animal has moved all four legs from one location to |
| | another |
| Rearing | animal has raised up on two hind legs |
| Head-Up Sniff | animal's head is raised while sniffing |
| Head-Down Sniff | animal's head is lowered while sniffing |
| Head Bobbing | animal rhythmically moving head up and down |
| Bite | animal repeatedly bites anywhere on body |
| Grooming | animal grooms body |
| Yawn | animal yawns |
| Scanning | animal visually searches while moving head to the side |
| Scratching | animal scratches anywhere on body |
| Licking | animal licks anywhere on body |
| Lying Down | animal lies down, but is not asleep |

Drug Treatment: Recombinant Tat (1-72) was produced as described in Ma & Nath, (1997). Tat (1-72) dose-dependently (1, 5, and 50 μg) produces striatal toxcicity in rats (Bansal et al., 2000; Aksenov et al., 2001; Aksenov et al., 2003). The highest dose of Tat (50 μg) induces sub-lethal histopathological changes in the striatum as there were no Tat-induced “clinical” outcomes such as convulsions, tremors, or morbidity (Aksenov et al., 2003). Rats in the present study were bilaterally injected with 15 μg of dose of Tat.

The cocaine treatment was administered as a bolus injection delivered in a volume of 1 ml/kg body weight (15 s), and was followed by flushing (15 s) with 0.2 ml heparinized (2.5%) saline (i.e., the approximate volume of the catheter). The dose of cocaine hydrochloride (3.0 mg/kg/day; Sigma, St. Louis, Mo.) was calculated on the weight of the salt and dissolved in saline immediately prior to injection in a volume of 1 ml/kg. The injection rate used was within the duration shown to produce moderate-to-robust behavioral sensitization to psychostimulant drugs (Wallace et al., 1996; Booze et al., 1999a; Booze et al., 1999b; Samaha et al., 2002; Harrod et al., 2004; 2005a; 2005b). The IV dosing regimen used in the present experiment has been shown to produce arterial drug concentrations and a pharmacokinetic profile in rats comparable to levels demonstrated in human cocaine volunteers (Booze et al., 1997; Evans et al., 1996), and has been associated with producing reinforcing and euphoric effects in humans.

Data Analysis: The automated data were analyzed using analysis of variance (ANOVA) techniques (BMDP statistical Software, 1990; SPSS, 2003; Winer, 1971). A mixed factorial ANOVA, with protein and drug as between-subjects factors, and day and time as within-subjects factors, was conducted for total activity and time spent in centermost portion of the compartment. Planned comparisons were conducted where appropriate.

Chi square (χ²) tests were used to compare incidence scores between groups on the data derived from the observational time sampling procedure. Incidence refers to the occurrence or nonoccurrence, rather than the frequency, of a particular behavior. Incidence scores were derived by summing observations of a particular behavior, for example rearing, for a specific rat across six observations. Thus, the rearing incidence for each rat was summed across rats in a particular group. The incidence data are nominal and between subjects in nature and therefore were analyzed using the χ² test. Scores below 5 are not reliably measured with the χ² statistic (Siegel and Castellan, 1988). Therefore, composite scores were created to increase incidence scores, and therefore maintain statistical accuracy. Locomotor, head up/down, rearing, and scratching incidence scores were combined for a locomotor composite, and grooming, licking, and washing incidence were summed for the orofacial composite. A Yates correction procedure was used with the χ² test. To further ensure statistical accuracy, a Bonferroni correction for multiple comparisons was used on the observed data. Thus, 12 comparisons were made for the locomotor and orofacial data separately, yielding a significance level of α≦0.004 (i.e., 0.05/12). Fourteen rats were excluded from data analysis: 6 rats exhibited leaking or clogged IV catheters, whereas 8 animals received Tat or VEH injections outside of the nucleus accumbens region. These exclusions produced the following sample sizes: VEH-SAL n=14; Tat-SAL n=10; VEH-COC n=13; and TAT-COC n=13.

### Results

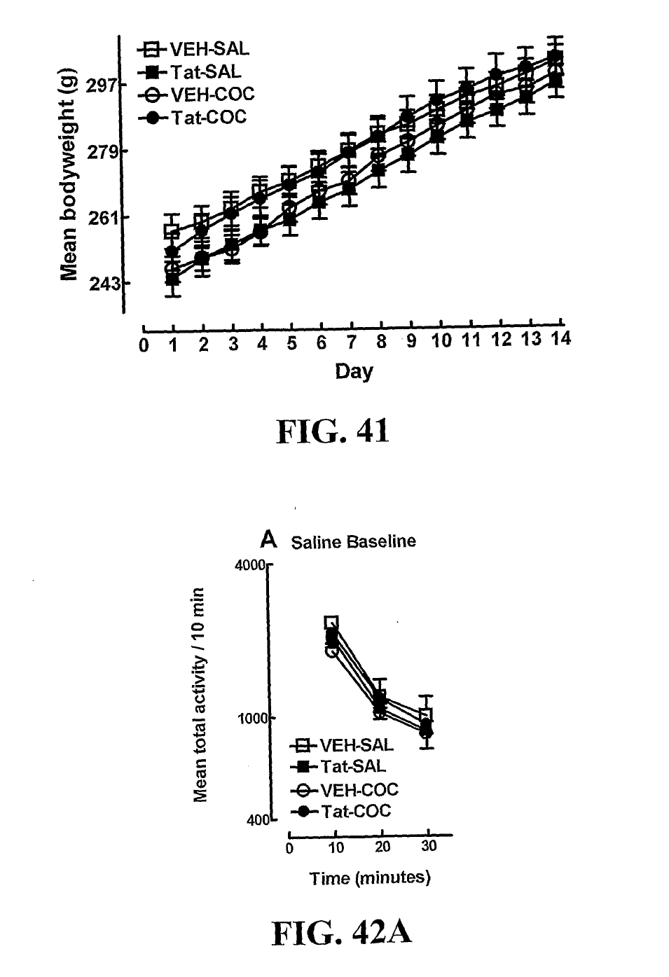

Bodyweight: FIG. 41 illustrates the mean bodyweight for rats in each group across the 14-day injection period following intra-accumbal Tat microinjection, and shows that all rats exhibited similar weights and gained weight at the same rate. A day×protein×drug ANOVA revealed a main effect of day [F(1, 53)=1594.3, p<0.001] which indicates that bodyweight increased over the 14-day experiment. No other main effects or interactions were significant, indicating Tat microinjection did not impair the general health of the animals.

Histology: Analysis of Nissl-stained sections through the nucleus accumbens region confirmed injection into the core region of the nucleus accumbens. Injection tracks outside of the nucleus accumbens were recorded from eight rats and these subjects were excluded from data analysis. No gross toxicity (e.g., no lesion) was present in the nucleus accumbens of Tat injected animals. However, increased numbers of reactive astrocytes were present in the area immediately adjacent to the injection track, similar to that previously reported following striatal Tat microinjections (Aksenov et al., 2003). Little or no reactivity was observed in saline injected animals.

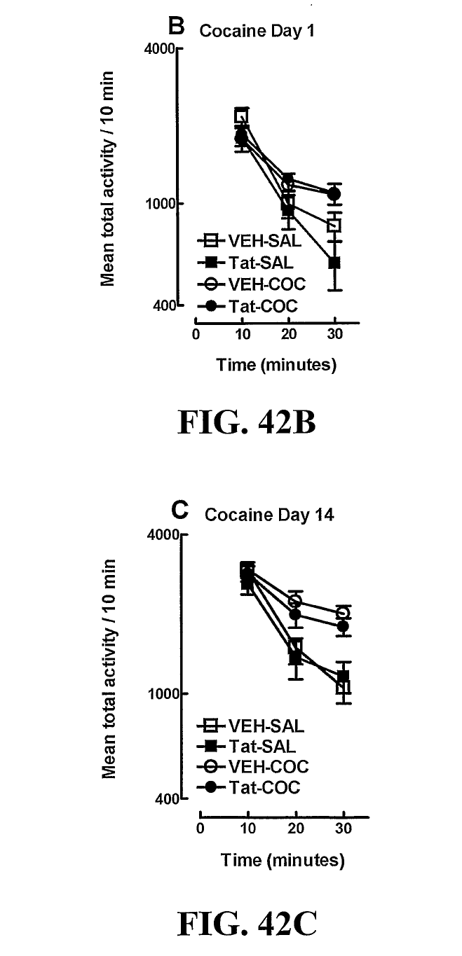

Automated Locomotor Activity: Total Activity: The total activity for the saline baseline measure, acute and repeated cocaine are illustrated in FIG. 42 A-C. A day×time×protein×drug ANOVA revealed a main effect of day [F(1, 46)=88.5, p<0.001], time [F(1, 46)=98.3, p<0.001], drug [F(1, 46)=7.1, p<0.05], and significant time×drug [F(1, 46)=5.8, p<0.05], and day×time [F(1, 46)=19.2, p<0.001] interactions. Overall, animals repeatedly injected with cocaine or saline exhibited decreased total activity across the 30-min session; however, rats injected with cocaine showed more activity than rats administered IV saline. The mean total activity (±SEM) over the 10, 20, and 30 minute-sessions was 2363.0 (±120.0), 1660.6 (±103.8), 1494.01 (±66.5) for rats injected with cocaine, and 2378.6 (±126.6), 1199.5 (±109.6), 901.4 (±70.2) for the animals injected with saline. These data indicate that repeated IV cocaine induced behavioral sensitization of total activity in ovariectomized rats.

A time×protein×drug ANOVA conducted for the acute (Day 1) data revealed a significant main effect of time [F(1, 50)=77.5, p<0.001], a time×drug [F(1, 50)=6.8, p<0.001], and time×protein [F(1, 50)=4.3, p<0.001] interaction. The time×protein×drug interaction was not significant. Comparison between the Tat-SAL and VEH-SAL groups did not reveal a significant difference [F (1, 24)=2.7, p>0.05]. These data indicate that acute cocaine did not increase total activity. Furthermore, Tat did not attenuate total activity in rats treated with intra-accumbal Tat and injected with SAL.

A time×protein×drug ANOVA conducted for the repeated (Day 14) data revealed a significant main effect of time [F(1, 46)=47.3, p<0.001], drug [F(1, 46)=9.5, p<0.01], and a significant time×drug [F(1, 46)=26.9, p<0.001], and time×protein×drug [F(1, 46)=4.8, p<0.001] interaction. The main effect of drug and the time×drug interaction indicates that rats administered IV cocaine demonstrated more total activity than rats injected with saline, consistent with the development of sensitization. Planned comparisons revealed that the total activity exhibited by Tat-COC and VEH-COC groups were not statistically different. Furthermore, there were no differences between the VEH-SAL and Tat-SAL groups.

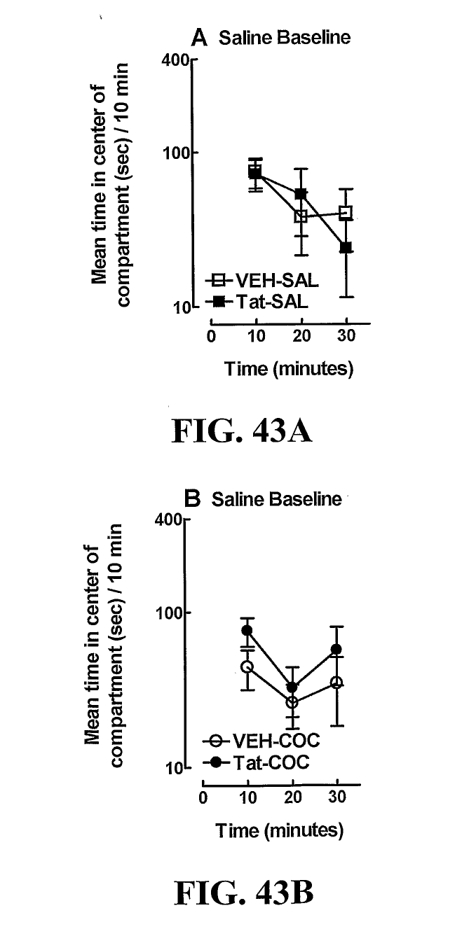

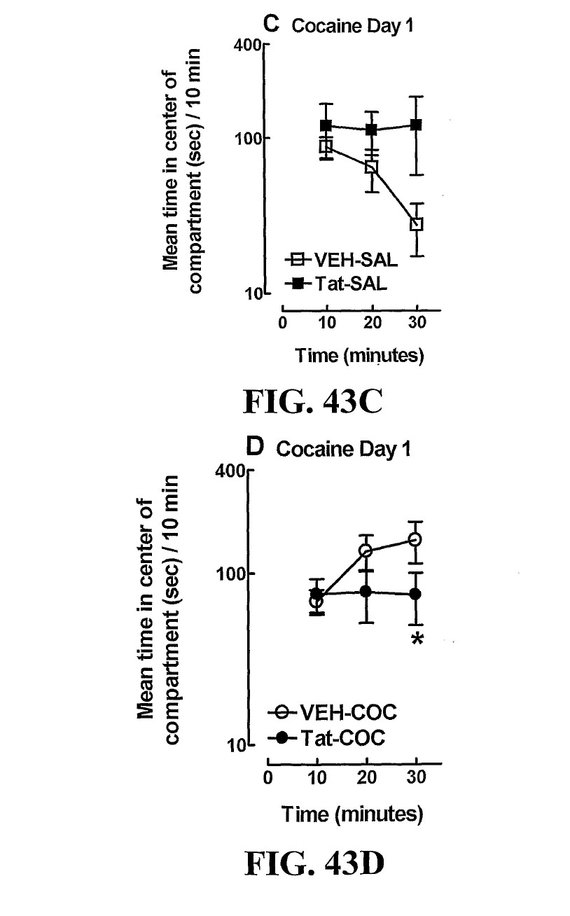

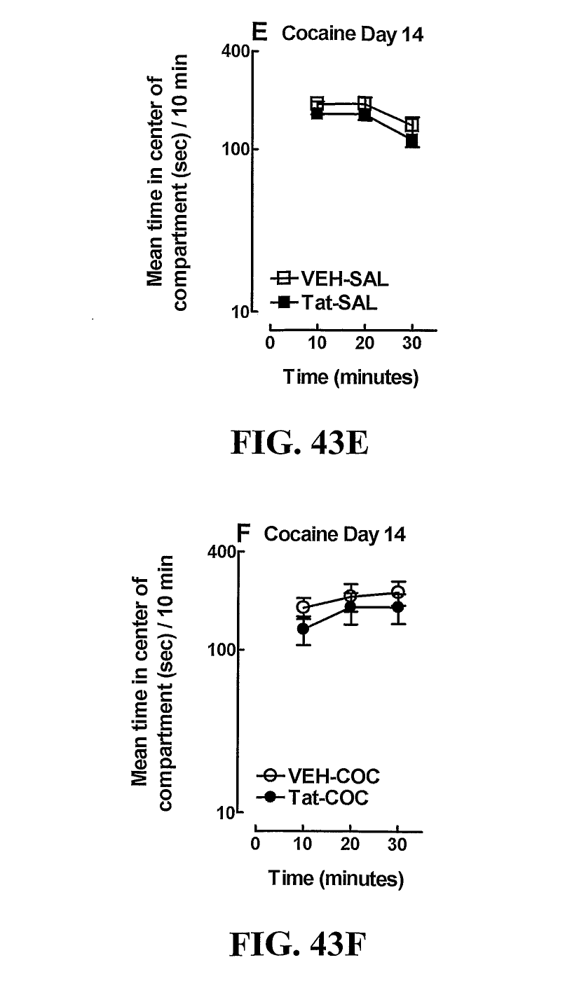

Centrally directed behavior: Mean TIC for the saline baseline measure, acute and repeated cocaine are presented in FIG. 43 A-F. A day×time×protein×drug ANOVA revealed a main effect of day [F(1, 46)=29.0, p<0.001], time [F(1, 46)=4.9, p<0.05], and a time×drug [F(1, 26)=18.4, p<0.000] interaction. The time×drug interaction further indicates that TIC changed over the 30-minute session depending on if the rats were injected with cocaine or SAL. Animals repeatedly injected with cocaine showed increased time in the center portion of the chamber, whereas rats injected with saline exhibited decreased time in the center. The mean TIC across the 10, 20, and 30-minute sessions was 132.0 (±13.1), 129.8 (±21.2), 85.2 (±20.7), and 116.6 (±12.4), 152.3 (±20.1), 160.3 (±19.6) for the animals that were injected with saline and COC, respectively. These data indicate that repeated IV cocaine induced behavioral sensitization of TIC in ovariectomized rats.

A time×protein×drug ANOVA conducted for the acute (Day 1) data revealed a significant time×drug [F(1, 50)=7.0, p<0.001] and time×protein×drug [F(1, 50) 3.9, p<0.05] interaction. The time×drug interaction indicates that acute IV cocaine produced an increase in TIC across the 30-minute session [72.1 (±15.0), 100.2 (±19.0), 108.9 (±24.4)], whereas saline injection resulted in a decrease in TIC [108.6 (±16.2), 92.4 (±20.5), 68.5 (±26.4)]. A non-significant protein×time interaction indicates that rats injected with Tat exhibited centrally directed behavior that was similar to non-Tat treated groups across the 30-minute session. The time×protein×drug interaction, however, suggests that an interaction between Tat and cocaine resulted in decreased TIC in the Tat-COC rats. Specific planned comparisons indicate that Tat significantly disrupted the development of the cocaine-induced increase in activity [F(1, 50)=4.3, p<0.05]. These patterns of behavior are illustrated in FIGS. 43C and 43D.

A time×protein×drug ANOVA conducted for the repeated (Day 14) data revealed a significant main effect of time [F(1, 46)=4.0, p<0.05] and a time×drug [F(1, 46)=12.8, p<0.01] interaction. The time×protein×drug interaction did not reach statistical significance. The time×drug interaction in the absence of a time×protein×drug interaction indicates that on day 14, IV cocaine increased centrally directed behavior over the 30-minute session, and Tat injection did not alter cocaine effects. These findings are illustrated in FIGS. 43E and 43F.

Observational Data: A modified version of the observational time sampling method first described by Fray et al. (1980) was used in the present experiment to examine the behaviors that rats exhibited following acute and repeated IV cocaine administration.

Effects of Tat and IV cocaine on locomotor composite incidence scores: Chi square analyses revealed that acute (day 1) cocaine injection did not produce significantly higher locomotor composite incidence scores in the VEH-COC rats compared to the VEH-SAL group [χ² (1)=2.3, p>0.05]. Tat-COC animals were not significantly different from the VEH-COC [χ² (1)=3.8, p>0.004] or Tat-SAL rats [χ² (1)=0.2, p>0.05]. There was not a difference in composite incidence between the VEH-SAL and Tat-SAL groups (1)=0.7, p>0.05].

Repeated (day 14) cocaine injection induced significantly higher locomotor composite incidence scores in the VEH-COC rats compared to the VEH-SAL group [χ² (1)=25.4, p≦004]. The VEH-COC group exhibited significantly more locomotor incidence than the Tat-COC rats on day 14 [χ² (1)=4.2, p>0.004]. The VEH-COC, and Tat-COC groups exhibited a significant increase in locomotor incidence between days 1 and 14 [χ² (1)=15.2, p<0.001; χ² (1)=14.4, p<0.001, respectively]. This increase in locomotor incidence across days 1-14 suggests that IV cocaine induced behavioral sensitization of locomotor incidence in the cocaine treated groups. There were no other significant differences on Day 14 (all p>0.004).

Effects of Tat and IV cocaine on orofacial composite incidence scores: Chi square analyses indicate that there were no differences between groups following acute or repeated IV cocaine injection (p>0.004). Moreover, none of the groups' orofacial composite incidence significantly changed from day 1 to day 14 (p>0.004).

### Example 15

### Dopaminergic marker proteins in the substantia nigra of HIV1-infected brains

Tyrosine hydroxylase (TH) enzyme activity, DAT-mediated DA uptake and ligand binding, as well as levels of the expression of DAT and TH proteins, are well-established measures in studies of dopamine (DA) transmission or dysfunction. While abnormalities in these DA markers have been thoroughly investigated in other subcortical dementias, little is known about changes in components of DA transmission systems in the brain of HIV patients. In the current study, levels of the dopamine (DA) related proteins, DAT and TH, were compared in extracts prepared from the substantia nigra (SN) of HIV positive brain tissue and seronegative controls, in order to obtain evidence for dopamine dysfunction in human HIV-1 infected brains.

All brain samples used in the current study were obtained from symptomatic HIV-seropositive patients with neurological abnormalities. Despite the reported presence of neurological symptoms, no diagnosis of HIV dementia had been confirmed in the HIV-positive donors of the current study. Substantia nigra tissue blocks from 7 confirmed HIV-1 infected brains and 8 controls were obtained through the National Disease Research Interchange (NDRI) (Philadelphia, Pa.). HIV-related fatalities were identified and classified as part of ongoing case-control study of neuropathological symptoms, HIV testing, medical treatments, supplemental background information and autopsy findings (Table 8).

TABLE 8
Donor Data

| | | | | | | Drug |
|---|---|---|---|---|---|---|
| | COD | Age | Sex | Race | PMI | Abuse |
| HIV | | | | | | |
| Donor | | | | | | |
| H1 | AIDS related | 38 | M | W | 6 | + |
| H2 | ″ | 54 | M | AA | 12 | − |
| H3 | ″ | 43 | M | AA | 9 | + |
| H4 | Cardiac arrest | 40 | M | W | 20 | − |
| H5 | Cancer | 57 | M | W | 12 | − |
| H6 | Cardiac arrest | 40 | M | W | 15 | − |
| H7 | Cancer | 42 | M | AA | 18 | + |
| Control | | | | | | |
| Donor | | | | | | |
| C1 | Cardiac arrest | 59 | F | W | 16 | − |
| C2 | Cardiac arrest | 51 | M | W | 9 | + |
| C3 | Cardiac arrest | 49 | M | AA | 15 | + |
| C4 | Cardiac arrest | 62 | M | W | 12 | + |
| C5 | Cancer | 42 | M | W | 13 | − |
| C6 | GI bleeding | 57 | M | W | 8 | − |
| C7 | Cardiac arrest | 58 | M | W | 10 | − |
| C8 | MVA | 42 | F | W | 24 | − |

Background data for the 7 HIV1-infected donors and the 8 seronegative controls.
COD = cause of death.
Sex M = Male, F = Female.
Race AA = African American, W = White.
PMI = postmortem interval

All HIV patients were confirmed as HIV-1 seropositive. Additionally, three HIV subjects tested positive for hepatitis C. Neurological symptoms documented in detailed medical histories of HIV patients used in the study include anxiety, depression, increased confusion, headaches with lack of consciousness, seizure disorder, and mental incompetence. Age-matched controls were individuals without a history of neurological diseases, or systemic diseases affecting the brain. Subjects were matched on the basis of postmortem interval (PMI), age, and drug abuse status. Poly-drug abusers were excluded from study. Because of the difficulty of rapidly obtaining autopsy tissue from human brain and confirming HIV diagnosis, average PMI for HIV cases was 13+5 hours. Tissue with similar PMI has been used to measure dopamine transporters (Mash et al, 2002) without significant degradation of marker proteins. Upon autopsy, brain tissue blocks containing the substantia nigra were removed rapidly, immediately placed in liquid nitrogen, and shipped/stored at −80° C. until processed. Brain samples were homogenized in 10 mM HEPES buffer (pH 7.4) containing 0.35 M sucrose 137 mM NaCl, 4.6 mM KCl, 1.1 mM KH2PO4, 0.6 mM MgSO₄, and a proteinase inhibitor cocktail. Homogenates were centrifuged at 1,000 g for 10 min to remove debris from the resulting tissue extract. Protein concentration in the supernatant was determined by the Pierce BCA method using the microplate variant of the procedure. Microplates were read in Synergy HT plate reader (Bio-Tek, Winooski, Vt., USA).

The initial screening of the homogenates prepared from HIV-positive and control substantia nigra tissue blocks was performed by Western blotting. The pattern of immunoreactive protein bands produced by each type of primary antibody (anti-TH, anti-DAT, and anti-NSE) was tested for an appropriate molecular weight. To confirm the absence of nonspecific secondary antibody binding, blots were incubated without primary antibodies in presence of only the subsequent secondary antibody. Representative Western blots were prepared using randomly chosen sets of HIV-positive and control extracts. Six HIV and six control extract samples were loaded onto 14-well 8 cm×7 cm×1 mm 12.5% SDS polyacrylamide mini-gels. Equal amounts of total protein (10-30 μg) were added per well. Duplicate gels were prepared for each set of samples. One was stained with Coomassie Blue to confirm equality of protein load through different lanes and another was transferred to nitrocellulose membrane. All Western blot analyses were repeated at least twice.

Following the initial Western blotting screening of the extracts, simultaneous quantitative analysis of all HIV-positive and control substantia nigra extracts was performed using BioDot SF 36-well microfiltration unit (Bio-Rad). All HIV and control samples were blotted onto 0.45 μm nitrocellulose membrane in duplicates.

Western blots and slot-blots were blocked for at least 30 min in 3% albumin/PBST and then used for immunostaining. After incubation with primary antibodies against TH, DAT, or NSE, blots were washed three times with PBST and incubated with the subsequent secondary alkaline phosphatase (AP)-conjugated antibody. Blots were then washed with PBST and developed with BCIP/NBT substrate solution. Images of the blots were obtained using CCD video camera (Pixera Corporation, Los Gatos, Calif.) and analyzed with MCID M7 Gel lane analysis software (Imaging Research, St. Catherines, Ontario).

Data were analyzed using an ANOVA with 2 (disease state)×3(markers), with markers serving as a within-subjects factor. To avoid violation of the homogeneity of variance assumption, data was transformed. Subsequently an unpaired two sample T-test assuming equal variance was used for post-hoc analyses. Analysis of slot blots used the mean pixel density (arbitrary units) for the HIV and control group, whereas western blot analysis used mean percentage of mean control density (pixel density) for the HIV and control group. A significant interaction was detected between disease state and marker in the overall ANOVA [F(2,26)=6.02, p<0.01].

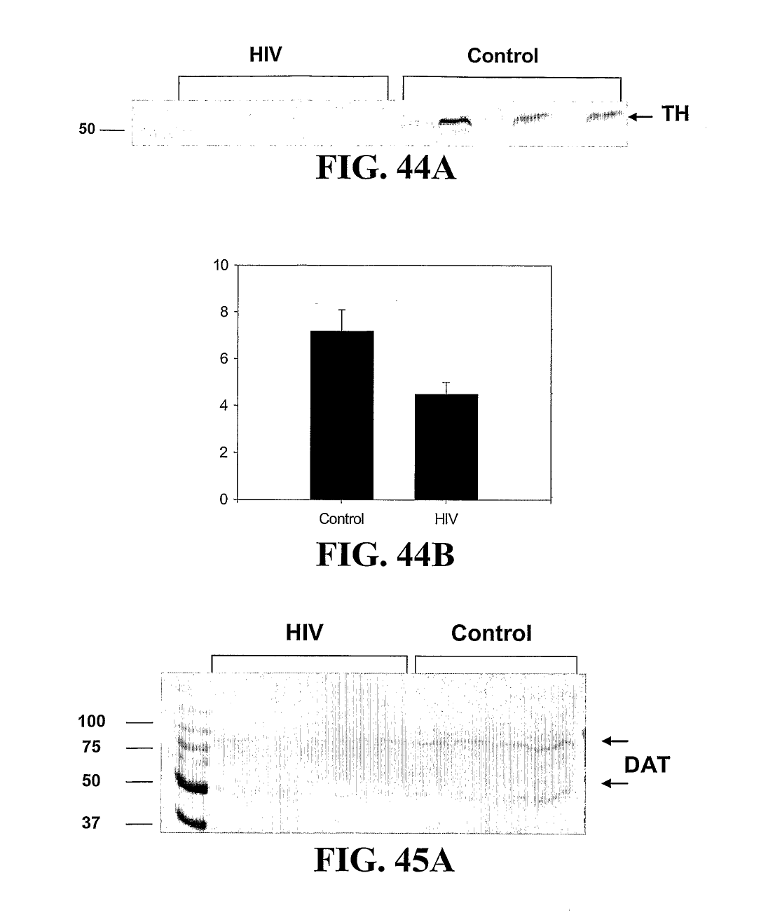

Immunostaining of blots for TH was performed using a mouse monoclonal anti-TH antibody generated against TH that has been isolated and purified from rat PC12 cells. The antibody is believed to have wide species cross-reactivity because it recognizes an epitope in the mid-portion of the TH molecule where extensive species homology exists (ImmuinoStar Inc, Hudson, Wis., USA). The primary antibody was diluted 1:2000 in 0.3% albumin/PBST and incubated with blots overnight at 4° C. Alkaline phosphatase-conjugated anti-mouse IgG was used as the secondary antibody (Sigma, working dilution 1:5000). Representative western blot which illustrates the banding pattern for the TH immunoreactivity is shown in FIG. 44A. The image of the blot displays TH immunoreactive protein bands of approximately 55 kD. The intensity of TH-positive bands was significantly (t(13)=−1.812, p<0.05) reduced in HIV1-infected tissue compared to seronegative controls. Results of the preliminary Western blot analyses were confirmed by simultaneous measurements of TH immunoreactivity in all HIV-infected and seronegative control tissue extracts using slot blot (t(13)=−2.817, p<0.01) FIG. 44B presents results of the slot blot analysis.

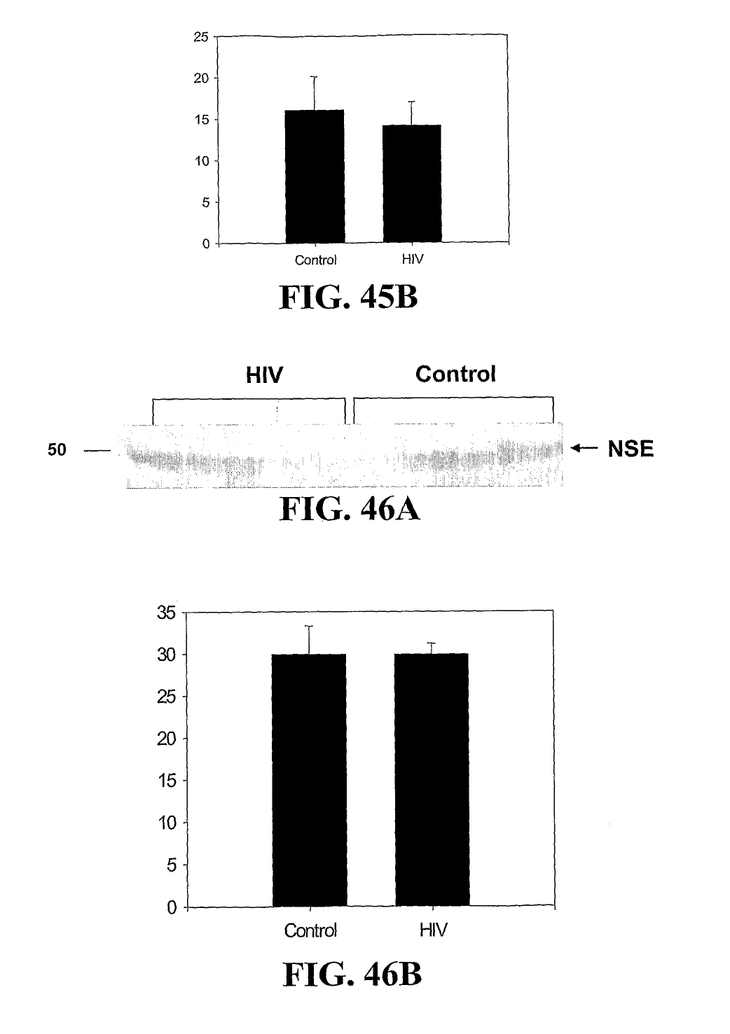

The plasma membrane protein DAT is considered to be a reliable marker of presynaptic dopaminergic terminal loss and immunological analysis of DAT protein provides sensitive means for investigation of molecular mechanisms of neurological disorders involving dopaminergic systems (Miller et al, 1997). The analysis of immunoreactive DAT protein levels in substantia nigra of HIV patients and control subjects was performed using a rabbit polyclonal antibody raised against amino acids 541-620 mapping at the C-terminus of the sodium-dependent dopamine transporter DAT of human origin (Santa-Cruz Inc, Santa Cruz, Calif., USA). The primary antibody was diluted 1:200 in 0.3% albumin/PBS-T. Blots were incubated with the primary antibody solution overnight at 4° C. Alkaline phosphatase-conjugated anti-rabbit IgG were used as the secondary antibody (Sigma, working dilution 1:2500). No significant reduction in DAT immunoreactivity in HIV1-infected tissue compared to seronegative controls was demonstrated by either the slot blot or western blot analysis (p>0.05 in both cases). FIG. 45A displays the qualitative banding pattern of DAT immunoreactivity with visible bands at approximately 50 kD and 70 kD, while FIG. 45B displays the quantitative analysis of the DAT slot blot.

Changes in NSE immunoreactivity are a recognized marker of neuronal cell damage in brain trauma and various neurodegenerative disorders (Blennow et al, 1994, Ding et al, 2000). Neuron-specific enolase (NSE) immunoreactivity levels were determined using a rabbit polyclonal anti-NSE antibody (Santa-Cruz Inc). The primary antibody was diluted 1:200 in 0.3% albumin/PBST. Alkaline phosphatase-conjugated anti-rabbit IgG were used as the secondary antibody (Santa-Cruz, working dilution 1:5000). There was no significant difference in NSE immunoreactivity in HIV1-infected tissue compared to seronegative controls for either the slot blot or western blot analysis (p>0.05 for both assays). FIG. 46A displays the qualitative western blot banding pattern of the NSE immunoreactivity, and FIG. 46B represents the western blot quantitative analysis of the NSE immunoreactivity.

Although virological studies indicate that the basal ganglia are a major target of HIV infection, the current study suggests that dopaminergic function located in SN can also be affected. The herein disclosed results demonstrate substantial changes in the expression of rate-limiting enzyme of dopamine metabolism, TH, in the substantia nigra of HIV patients with documented neurological abnormalities. Interestingly, although the immunoreactivity of TH was decreased, there was no concurrent alteration in either DAT protein levels or NSE protein levels in the substantia nigra of HIV-1 infected tissue. The absence of significant changes in DAT and NSE protein levels indicates that the observed decrease in TH protein levels is unlikely to result from death of dopaminergic neurons in the SN of HIV patients.

The current studies demonstrate decreased levels of tyrosine hydroxylase in substantia nigra of HIV patients. This decrease is not caused by selective cell loss of dopaminergic neurons, as there was no decrease in dopamine transporter protein levels or neuronal specific enolase. Decrease in TH is likely a precursor to more severe central nervous system alterations which develop with progression of HAD.

### REFERENCES

Adamson, D. C., Dawson, T. M., Zink, M. C., Clements, J. E., Dawson, V. L. (1996) Mol. Med. 2, 417-428.
Adamson, D C, Dawson, T M, Zink, M C, Clements, J E, Dawson, V L. (1996) Neurovirulent simian immunodeficiency virus infection induces neuronal, endothelial, and glial apoptosis. Mol. Med.; 2: 417-428.
Aguila-Mansilla, N., Little, B. B., Ho, R. H., Barnea, A. (1997) Differential potencies of cocaine and its metabolites, cocaethylene and benzoylecgonine, in suppressing the functional expression of somatostatin and neuropeptide Y producing neurons in cultures of fetal cortical cells. Biochem Pharmacol. 54, 491-500.
Aksenov M Y, Aksenova M V, Nath A, Ray P D, Mactutus C F, Booze R M. (2005) Cocaine-mediated enhancement of Tat toxicity in rat hippocampal cell cultures: The role of oxidative stress and D1 dopamine receptor. Neurotoxicology In press.
Aksenov M Y, Hallelrot U, Bansai A K, Wu G, Nath A, Anderson C, Mactutus C, Booze R M. 2001. Oxidative damage induced by the injection of HIV-1 Tat protein in the rat striatum. Neurosci Lett 305:5-8.
Aksenov M Y, Hallelrot U, Wu G, Nath A, Anderson C, Mactutus C, Booze R M. 2003. Temporal relationships between HIV-1 Tat-induced neuronal degeneration, OX-42 immunoreactivity, reactive astrocytosis, and protein oxidation in the rat striatum. Brain Res 987:1-9.
Aksenov M Y, Hasselrot U, Bansal A K, Wu G, Nath A, Anderson C, Mactutus C F, Booze R M (2001) Oxidative damage induced by the injection of HIV-1 Tat protein in the rat striatum. Neurosci Lett 305:5-8.
Aksenov M Y, Hasselrot U, Wu G, Nath A, Anderson C, Mactutus C F, Booze R M (2003) Temporal relationships between HIV-1 Tat-induced neuronal degeneration, OX-42 immunoreactivity, reactive astrocytosis, and protein oxidation in the rat striatum. Brain Res.; 987: 1-9.
Aksenov M Y, Hasselrot U, Wu G, Nath A, Anderson C, Mactutus C F, Booze R M (2003) Temporal relationships between HIV-1 Tat-induced neuronal degeneration, OX-42 immunoreactivity, reactive astrocytosis, and protein oxidation in the rat striatum. Brain Res 987:1-9.
Aksenov, M. Y, Tucker, H. M, Nair P, Aksenova, M. V, Butterfield, D. A, Estus, S., Markesbery, W. R. (1998) The expression of key oxidative stress handling genes in different brain regions of Alzheimer's disease patients. J Mol. Neurosci. 11, 151-164.
Aksenov, M. Y., Aksenova, M. V., Butterfield, D. A., Geddes, J. W., Markesbery, W. R (2001) Protein oxidation in the brain in Alzheimer's disease. Neuroscience, 103, 373-383.
Aksenova M, Aksenov M Y, Nath A, Mactutus C, Booze R. Cocaine enhances oxidative stress-dependent toxicity of HIV-1 Tat in rat hippocampal cell cultures. Society for Neuroscience 573. 8. 2004.
Aksenova M J, Aksenov M, Mactutus C F, Booze R M (2005) Cell culture models of oxidative stress and injury in the central nervous system. Current Neurovasc. Res 2(1):73-89.
Aksenova M J, Aksenov M, Nath A, Ray P D, Mactutus C F, Booze R M (2005) Cocaine enhances Tat-mediated toxicity and intracellular free radical production in rat hippocampal cell culture. Neurotoxicology in press.
Aksenova, M. J., Aksenov, M., Mactutus, C. F., Booze, R. M. (2005) Cell culture models of oxidative stress and injury in the central nervous system. Current Neurovasc. Res., in press. Review
Aksenova, M. J., Aksenov, M., Nath, A., Ray, P. D., Mactutus, C. F., Booze, R. M. (2005) Cocaine enhances Tat-mediated toxicity and intracellular free radical production in rat hippocampal cell culture. Free Radic. Biol. Med., provisionally accepted.
Aksenova, M. V., Aksenov M. Y., Markesbery W. R., Butterfield D. A. (1999) Aging in a dish: age-dependent changes of neuronal survival, protein oxidation, and creatine kinase BB expression in long-term hippocampal cell culture. J. Neurosci. Res. 58, 308-317.

Anagnostaras S G, Robinson T E (1996) Sensitization to the psychomotor stimulant effects of amphetamine: modulation by associative learning. Behav Neurosci 110(6):1397-1414.
Annunziata P. (2003). Blood-brain barrier changes during invasion of the central nervous system by HIV-1. Old and new insights into the mechanism. Journal of Neurology, 250, 901-906.
Arendt, G. (2005) Neurological Manifestations of HIV-Infection in the Era of Highly Active Antiretroviral Therapy (HAART). Fortschr Neurol Psychiatr; 73: 577-586.
Arnold, H. M., Fadel, J., Sarter, M. and Bruno, J. P. (2001) Amphetamine-stimulated cortical acetylcholine release: role of the basal forebrain. Brain Res. 894: 74-87.
Asthana S, Baker L, Craft S, Stanczyk F, Veith R, Raskind M et al.: High-dose estradiol improves cognition for women with AD. Neurology 2001, 57: 605-612.
Ba F, Pang P K T, Benishin C G. 2004a. The role of Ca2+ channel modulation in the neuroprotective actions of estrogen in □-amyloid protein and 1-methyl-4-phenyl-1,2,3,6-tetrahydropyridine (MPTP) cytotoxic models. Neurochem Int 45:31-38.
Ba F, Pang P K T, Davidge S T, Benishin C G. 2004b. The neuroprotective effects of estrogen in SK-N-SH neuroblastoma cell cultures. Neurochem Int 44:401-411.
Bagetta G, Corasaniti M T, Paoletti A M, Berliocchi L, Nistico R, Giammarioli A M, Malorni W, Finazzi-Agro A. 1998. HIV-1 gp120-induced apoptosis in the rat neocortex involves enhanced expression of cyclo-oxygenase type2 (COX-2). Biochem Biophys Res Comm 244:819-824.
Bagetta G, Piccirilli S, Del Duca C, Morrone L A, Rombolá L, Nappi G et al.: Inducible nitric oxide synthase is involved in the mechanisms of cocaine enhanced neuronal apoptosis induced by HIV-1 gp120 in the neocortex of rat. Neurosci Lett 2004, 356: 183-186.
Banisadr G, Dicou E, Berbar T, Rostene W, Lombet A, Haour F. 2000. Characterization and visualization of [12511] stromal cell-derived factor-1□ binding to CXCR4 receptors in rat brain and human neuroblastoma cells. J Neuroimmunol 110:151-160.
Bansal A. K, Mactutus C F, Nath A, Maragos W, Hauser I K, Booze R M. 2000. Neurotoxicity of HIV-1 proteins gp120 and Tat in the rat striatum. Brain Res 879:42-49.
Bansal A K, Mactutus C F, Nath A, Maragos W, Hauser K F, Booze R M (2000) Neurotoxicity of HIV-1 proteins gp120 and Tat in the rat striatum. Brain Res 879: 42-49.
Bansal, A. K., Mactutus, C. F., Nath, A., Maragos, W., Hauser, K. F., Booze, R. M. (2000) Neurotoxicity of HIV-1 proteins gp120 and Tat in the rat striatum. Brain Res. 879, 42-49.
Barak O, Weidenfeld J, Goshen I, Ben-Hur T, Taylor A N, Yirmiya, R (2002). Intracerebral HIV-1 glycoprotein 120 produces sickness behavior and pituitary-adrenal activation in rats: Role of prostaglandins, Brain Behavior, and Immunity, 16:720-735.
Barks J D E, Liu X-H, Sun R, & Silverstein F S. (1997). Gp120, a human immunodeficiency virus-1 coat protein, augments excitotoxic hippocampal injury in perinatal rats. Neuroscience, 76(2), 397-409.
Becker J B, Molenda M, Hummer DL (2001) Gender differences in the behavioral responses to cocaine and amphetamine: Implications for mechanisms mediating gender differences in drug abuse. Ann N Y Acad Sci 937:172-187.
Behl C, Skutella T, Lezoualc'h F, Post A, Widman M, Newton C et al.: Neuroprotection against oxidative stress by estrogens: structure-activity relationship. Mol Pharmacol 1997, 51: 535-541.
Behnisch T, Francesconi W, Samna PP (2004). HIV secreted protein Tat prevents long-term potentiation in the hippocampal CA1 region. Brain Research, 1012:187-189.
Bell J E, Brettle R P, Chiswick A, Simmonds P: HIV encephalitis, proviral load and dementia in drug users and homosexuals with AIDS. Effect of neocortical involvement. Brain 1998, 121: 2043-2052.
Bell J E, Donaldson Y K, Lowrie S, McKenzie C A, Elton R A, Chiswick A, Brettle R P, Ironside J W, Simmonds P (1996) Influence of risk group and zidovudine therapy on the development of HIV encephalitis and cognitive impairment in AIDS patients. AIDS 10(5):493-499.
Bell, J. E., Donaldson, Y. K., Lowrie, S., McKenzie, C. A., Elton, R. A., Chiswick, A., Brettle, R. P., Ironside, J. W., Simmonds, P. (1996) Influence of risk group and zidovudine therapy on the development of HIV encephalitis and cognitive impairment in AIDS patients. AIDS. 10(5):493-9.
Belman A L, Diamond G, Dickson D, Horoupian D, Llena J, Lantos G, & Rubinstein A. (1988). Pediatric acquired immunodeficiency syndrom. American Journal of Diseases of Children, 142, 29-35.
Belman A L. (1997). Infants, children, and adolescents. In J R Berger and R M Levy (Eds). AIDS and the Nervous System, Second Edition. Lippincott-Raven Publishers, pp. 223-253.
Bennet B, Hyde C, Pecora J, Clodfelter J: Long-term cocaine administration is not neurotoxic to cultured fetal mesencephalic dopamine neurons. Neurosci Lett 2003, 153: 210-214.
Bennett, B. A., Rusyniak, D. E. and Hollingsworth, C. K. (1995) HIV-1 gp120-induced neurotoxicityto midbrain dopamine cultures. Brain Res. 705:168-76.
Benveniste H and Huttemeier P C (1990) Microdialysis: Theory and application. Prog. Neurobiol. 35:195-215.
Berger J R, Arendt G. (2000) HIV dementia: the role of the basal ganglia and dopaminergic systems. Journal of Psychopharmacology; 14: 214-221
Berger J R, Nath A, Greenberg R N, Andersen A H, Greene R A, Bognar A et al.: Cerebrovascular changes in the basal ganglia with HIV dementia. Neurology 2000, 54: 921-926.
Berger, J. R. and Nath, A. (1997). HIV dementia and the basal ganglia. Intervirology 40, 122-31.
Biedler J L, Helson L, Spengler B A. 1973. Morphology and growth, tumorigenicity, and cytogenetics of human neuroblastoma cells in continuous culture. Cancer Res 33:2643-2652.
Blanchard-Fillion B, Souza J M, Friel T, Jiang G C, Vrana K, Sharov V et al. (2001) Nitration and inactivation of tyrosine hydroxylase by peroxynitrite. J. Biol Chem; 279: 46017-46023.
Blanche S, Mayaux M-J, Rouziox C, Teglas J-P, Firtion G, Monpoux F, Cicaru-Vigneron N, Meier F, Tricoire J, Courpotin C, Vilmer E, Griscelli C, & Delfraissy J-F. (1994). Relation of the course of HIV infection in children to the severity of the disease in their mothers at delivery, New England Journal of Medicine, 330(5), 308-312.
Blennow K, Wallin A, Eliman R. (1994) Neuron specific enolase in cerebrospinal fluid: a biochemical marker for neuronal degeneration in dementia disorders? J Neural Transm
Parl Dis Dement Sect; 8: 183-91. BMDP Statistical Software (1990) University of California Press, Berkeley, Calif.
Bonavia R, Bajetto A, Barbero S, Albini A, Noonan D M, Schettini G: HIV-1 Tat causes apoptotic death and calcium homeostasis alterations in rat neurons. Biochem Res Commun 2001, 288: 301-308.
Booze R M, Lehner A F, Wallace D R, Welch M A, Mactutus C F (1997) Dose-response cocaine pharmacokinetics and metabolite profile following intravenous administration and arterial sampling in unanesthetized, freely moving male rats. Neurotoxicol Teratol 19(1):7-15.
Booze R M, Welch M A, Wood M L, Billings K A, Apple S R, Mactutus C F (1999a) Behavioral sensitization following repeated intravenous nicotine administration: gender differences and gonadal hormones. Pharmacol Biochem Behav 64(4):827-839.
ooze R M, Wood M L, Welch M A, Berry S, Mactutus C F (1999b) Estrous cyclicity and behavioral sensitization in female rats following repeated intravenous cocaine administration. Pharmacol Biochem Behav 64(3):605-610.
Booze, R. M. and Wallace, D. R. (1995) Dopamine D2 and D3 receptors in the rat striatum and nucleus accumbens: Use of 7-OH-DPAT and [¹²⁵I]-Iodosulpride. Synapse, 19:1-13.
Booze, R. M., Crisostomo, E. A. and Davis, J. N. (1989) Species differences in the localization and number of CNS beta-adrenergic receptors: Rat vs. guinea pig. J. Pharm. Exp. Ther., 249:911-920.
Booze, R. M., Lehner, A. F., Wallace, D. R., Welch, M. A., and Mactutus, C. F. (1997) Dose-response cocaine pharmacokinetics and metabolite profile following intravenous administration and arterial sampling in unanesthetized freely moving male rats. Neurotoxicol. Teratol., 19:7-15.
Booze, R. M., Welch, M. A., Wood, M. L., and Mactutus, C. F. (1999) Behavioral sensitization and estrus cyclicity in female rats following repeated intravenous cocaine administration. Pharmacol. Biochem. Behav. 64: 605-610.
Bosse R, Rivest R, Di Paolo T. (1997) Ovariectomy and estradiol treatment affect the dopamine transporter and its gene expression in the rat brain. Mol. Brain. Res. 46:343-346.
Bouwman F H, Skolasky R L, Hes D, Selnes O A, Glass J D, Nance-Sproson T E et al.: Variable progression of HIV-associated dementia. Neurology 1998, 50: 1814-1820.
Boven L A, Gomes L, Hery C, Gray F, Verhoef J, Portegies P et al. (1999) Increased peroxynitrite activity in AIDS dementia complex: implications for the neuropathogenesis of HIV-1 infection. Journal of Immunology; 162: 4319-4327.
Boven L A, Gomes L, Hery C, Gray F, Verhoef J, Portegies P, Tardieu M, Nottet H S L M. 1999. Increased peroxynitrite activity in AIDS dementia complex: Implications for the neuropathogenesis of HIV-1 infection. J Immunol 162:4319-4327.
Brenneman D E, Westbrook G L, Fitzgerald S P, Ennist D L, Elkins K L, Ruff M R, Pert C B (1988). Neuronal cell killing by envelope protein of HIV and its prevention by vasoactive intestinal peptide. Nature, 335:639-642.
Brenneman, D. E., Westbrook, G. L., Fitzgerald, S. P., Ennist, D. L., Elkins, K. L., Ruff, M. R., Pert, C. B. Neuronal cell killing by the envelope protein of HIV and its prevention by vasoactive intestinal peptide, Nature 335 (1988) 639-642.
Brew B J, Rosenblum M, & Price R W. (1988). AIDS dementia complex and primary HIV brain infection, Journal of Neuroimmunology, 20, 133-140.
Brew, B. J., Rosenblum, M., Price, R. W. AIDS dementia complex and primary HIV brain infection, J Neuroimm 20 (1988) 133-140.
Brewer G. J., Torricelli J. R., Evege E. K., Price P. J. (1993) Optimized survival of hippocampal neurons in B27-supplemented neurobasal, a new serum-free medium combination. J. Neurosci. Res., 35, 567-576.
Brooke S M, Howard S A, Sapolsky R M. 1998. Energy dependency of glucocorticoid exacerbation of gp120 neurotoxicity. J Neurochem 71:1187-1193.
Brooke S M, McLaughlin J R, Cortopassi K M, Sapolsky R M. 2002. Effect of gp120 on glutathione peroxidase activity in cortical cultures and the interaction with steroid hormones. J Neurochem 81:277-284.
Brooke S M, Sapolsky R M. 2000. The effects of steroid hormones in HIV-related neurotoxicity: A mini review. Biol Psychiatry 48:881-893.
Brooke, S., Chan, R., Howard, S, and Sapolosky, R. (1997) Endocrine modulation of the neurotoxicity of gp120: implications for AIDS-related dementia complex. Proc Natl Acad Sci USA, 94:9457-9462.
Bruce-Keller A J, Chauhan A, Dimayuga F O, Gee J, Keller J N, & Nath A. (2003). Synaptic transport of human immunodeficiency virus-Tat protein causes neurotoxicity and gliosis in rat brains. The Journal of Neuroscience, 23(23), 8417-8442.
Bubien J K, Benveniste E N, Benos D J. 1995. HIV-gp120 activates large-conductance apamin-sensitive potassium channels in rat astrocytes. Am J Physiol (Cell Physiol) 37:C1440-C1449.
Buchanan R, Wang S, Huang C: Analyses of nursing home residents with HIV and dementia using the minimum data set. JAIDS 2001, 26: 246-255.
Bussiere J L, Hardy L M, Peterson M, Foss J A, Garman R H, Hoberman A M, Christian M S (1999). Lack of developmental Neurotoxicity of MN rgp120/HIV-1 administered subcutaneously to neonatal rats. Toxicological Science 48:90-99.
Butterfield D A, Stadtman E R. (1997) Protein oxidation processes in aging brain. Adv. Cell Aging Gerontol.; 2: 161-191.
Butterfield, D. A., Stadtman, E. R. (1997) Protein oxidation processes in aging brain. Adv. Cell Aging Gerontol. 2, 161-191.
Buzy J, Brenneman D E, Pert C B, Martin A, Salazar A, Ruff M R (1992). Potent gp120-like neurotoxic activity in the cerebrospinal fluid of HIV-infected individuals is blocked by peptide T. Brain Research, 598:10-18.
Caine S B, Geyer M A, & Swerdlow N R. (1991). Carbachol infusion into the dentate gyrus disrupts sensorimotor gating of the startle reflex in rats. Psychopharmacology, 105(3), 347-354.
Caine S B, Humby T, Robbins T W, & Everitt B J. (2001). Behavioral effects of psychomotor stimulants in rats with dorsal or ventral subiculum lesions: Locomotion, cocaine self-administration, and prepulse inhibition of startle. Behavioral Neuroscience, 115(4), 880-894.
Caine, S. B., Geyer, M. A., Swerdlow, N. R. Hippocampal modulation of acoustic startle and prepulse inhibition in the rat, Pharmacol Biochem Behav 43 (1992) 1201-1208.
Callier S, Morissette M, Grandbois M, Di Paolo T. (2000) Stereospecific prevention by 17-estradiol of MPTP-induced dopamine depletion in mice. Synapse. 37:245-251.
Callier S, Morissette M, Grandbois M, Pelaprat D, Di Paolo T. (2001) Neuroprotective properties of 17β-estradiol, progesterone, and raloxifene in MPTP C57B1/6 mice. Synapse. 41:131-138.
Carvelli L, Moron J A, Kahlig K M, et al. (2002) PI 2-kinase regulation of dopamine uptake. J. Neurochem. 81:859-869.
Castello, E., Baroni, N., Pallestrini, E. (1998). Neorotological auditory brain stem response findings in human immunodeficiency virus-positive patients without neurologic manifestations. Annals of Otolology, Rhinology & Laryngology, 107, 1054-60.
Catani M V, Corasaniti M T, Navarra M, Nistico G, Finazzi-Agro A, Melino G. 2000. gp120 induces cell death in human neuroblastoma cells through the CXCR4 and CCR5 chemokine receptors. J Neurochem 74:2373-2379.
Catani M V, Corasaniti M T, Ranalli M, Amantea D, Litovchick A, Lapidot A, & Melino G. (2003). The Tat antagonist neomycin B hexa-arginine conjugate inhibits gp-120-induced death of human neuroblastoma cells. Journal of Neurochemistry, 84, 1237-1245.
CDC (2002) HIV/AIDS Surveillance Report: Cases of HIV infection and AIDS in the United States. Vol. 14, Atlanta Ga.: US Department of Health and Human Services.
Chang, H. C., Samaniego, F., Nair, B. C., Buonaguro, L., Ensoli, B. (1997) HIV-1 Tat protein exits from cells via a leaderless secretory pathway and binds to extracellular matrix-associated heparan sulfate proteoglycans through its basic region. AIDS. 11(12):1421-31.
Chearskul S, Chotpitayasunondh T, Simonds R J, Wanprapar N, Waranawat N, Punpanich W, Chokephaibulkit K, Mock P A, Neeyapun K, Jetsawang B, Teeraratkul A, Supapol W, Mastro T D, Shaffer N. (2002). Survival, disease manifestations, and early predictors of disease progression among children with perinatal human immunodeficiency virus infection in Thailand. Pediatrics, 110(2), e25.
Cheng J, Nath A, Knudsen B, Hochman S, Geiger J D, Ma M, & Magnuson D S K. (1998). Neuronal excitatory properties of human immunodeficiency virus type 1 TAT protein. Neuroscience. 82(1), 97-106.
Chemecky C C, Berger B Je: Laboratory Tests and Diagnostic Procedures, 3 edn. Philadelphia: WB Saunders; 2001.
Chi L, Reith M E A. (2003) Substrate-induced trafficking of the dopamine transporter in heterologously expressing cells and in rat striatal synaptosomal preparations. J. Pharmacol. Exp. Therap. 307:729-736.
Chiesi A, Vella S, Dally L, Pedersen C, Danner S, Johnson A et al.: Epidemiology of AIDS dementia complex in Europe by the AIDS in Europe study group. J Acquir Immune Defic Syndr Hum Retrovirol 1996, 11: 39-44.
Chiu F, Rozental R, Bassallo C, Lyman W, Spray D: Human fetal neurons in culture: intercellular communication and voltage- and ligand-gated responses. J Neurosci Res 1994, 38: 687-697.
Ciriza I, Axcoitia I, Garcia-Segura LM: Reduced progesterone metabolites protect rat hippocampal neurones from kainic acid excitotoxicity in vivo. J Neuroendocrin 2004, 16: 58-63.
Clark R A, Bessinger R. 1997. Clinical manifestations and predictors of survival in older women infected with HIV. J Acquir Defic Syndr Hum Retroviol 15:341-345.
Codazzi F, Racchetti G, Grohovaz F, Meldolsi J. 1996. Transduction signals induced in rat brain cortex astrocytes by the HIV-1 gp120 glycoprotein. FEBS Lett 384:135-137.
Cohen, J. (1988) Statistical Power Analysis for the Behavioral Sciences. Lawrence Erlbaum Associates, Hillsdale, N.J.
Constantine G D, Pickar J H. 2003. Estrogens in postmenopausal women: recent insights. Curr Opin Pharmacol 3:626-634.
Corasaniti M T, Bagetta G, Rotiroti D, Nistico G. 1998. The HIV envelope protein gp120 in the nervous system: interactions with nitric oxide, interleukin-1□ and nerve growth factor signaling, with pathological implications in vivo and in vitro. Biochem Pharmacol 56:153-156.
Craig M C, Cutter W J, Wikham H, van Amelsvoort T A M J, Rymer J, Whitehead M, Murphy D G M. (2004) Effect of long-term estrogen therapy on dopaminergic responsivity in post-menopausal women—a preliminary study. Psychoneuroendocrinology. 29:1309-1316.
Crowley, W. R., O'Donohue, T. L., and Jacobowitz, D. M. (1978) Changes in catecholamine content in discrete brain nuclei during the estrus cycle of the rat. Brain Res. 147:315-326.
Culmsee C, Vedder H, Ravati A, Junker V, Otto D, Ahlemeyer B et al.: Neuroprotection by estrogens in a mouse model of focal cerebral ischemia and in cultured neurons: Evidence for a receptor-independent antioxidative mechanism. J Cereb Blood Flow Metab 1999, 19: 1263-1269.
Currie L J, Harrison M B, Trugman J M, Bennett J P, Wooten G F. 2004. Postmenopausal estrogen use affects risk for Parkinson Disease. Arch Neurol 61:886-888.
Czub, S., Czub, M., Koutsilieri, E., Sopper, S., Villinger, F., Muller, J. G., Stahl-Hennig, C., Riederer, P., terMeulen, V., Gosztonyi, G. (2004) Modulation of simian immunodeficiency virus neuropathology by dopmainergic drugs. Acta Neuropathol. 107:216-226.
Czub, S., Koutsilieri, E., Sopper, S., Czub, M., Stahl-Hennig, C., Muller, J. G., Pedersen, V., Gsell, W., Heeney, J. L., Gerlach, M., Gosztonyi, G., Riederer, P., ter Meulen, V. (2001) Enhancement of central nervous system pathology in early simian immunodeficiency virus infection by dopaminergic drugs. Acta Neuropathol (Berl). 101(2):85-91.
D'Astous, M. D., Morissette, M., DiPaolo, T. (2004) Effect of estrogen receptor agonists treatment in MPTP mice: evidence of neuroprotection by an ER-alpha agonist. Neuropharmacol. 47:1180-1188.
Da Cunha A, Mintz M, Eiden L E, & Sharer L R. (1997). A neuronal and neuroanatomical correlate of HIV-1 encephalopathy relative to HIV-1 encephalitis in HIV-1-infected children. Journal of Neuropathology and Experimental Neurology, 56(9), 974-987.
Daly R, Su T-P, Schmidt P, Pagliaro M, Pickar D, Ruiz-Lozano P: Neuroendocrine and behavioral effects of high-dose anabolic steroid administration in male normal volunteers. Psychoneuroendocrinology 2003, 28: 317-331.
Daniels, G. M., Amara, S. G. (1999) Regulated trafficking of the human dopamine transporter: clathrin-mediated internalization and lysosomal degradation in response to phorbol esters. J. Biol. Chem. 274:35794-35801.
Datla, K. P., Murray, H. E., Pillai, A. V., Gillies, G. E., Dexter, D. T. (2003) Differences in dopaminergic neuroprotective effects of estrogen during the estrous cycle. Neuroreport. 14:47-50.
Davies J, Everall I P, Weich S, McLaughlin J, Scaravilli F, Lantos P L (1997) HIV-associated brain pathology in the United Kingdom: an epidemiological study. AIDS 11: 1145-1150.
Davies J, Everall I P, Weich S, McLaughlin J, Scaravilli F, Lantos P L: HIV-associated brain pathology in the United Kingdom: an epidemiological study. AIDS 1997, 11: 1145-1150.
Dewhurst S, Gelbard H A, Fine S M. (1996) Neuropathogenesis of AIDS. Mol Med. Today.; 2:16-23.
Dewhurst, S., Gelbard, H. A., Fine, S. M. (1996) Neuropathogenesis of AIDS. Mol Med. Today. 2, 16-23.
Di Paolo, T., Falardeau, P., and Morrissette, M. (1988) D2 dopamine agonist binding sites fluctuate during the rat estrous cycle. Life Sci. 43:665-672.
Ding M, Haglid K G, Hamberger A. (2000) Quantitative immunochemistry on neuronal loss, reactive gliosis and BBB damage in cortex/striatum and hippocampus/amygdale after systemic kainic acid administration. Neurochem Int; 36: 313-318.
Dluzen D E, Park J H, Kim K. (2002) Modulation of olfactory bulb tyrosine hydroxylase and catecholamine transporter mRNA by estrogen. Brain Res. Mol. Brain. Res. 108:121-128.
Dluzen D E. (2000) Neuroprotective effects of estrogen upon the nigrostriatal dopaminergic system. J. Neurocytol. 29:387-399.
Doolen S, Zahniser N R. (2002) Conventional protein kinase C isoforms regulate human dopamine transporter activity in Xenopus oocytes. FEBS Lett. 516:187-190.
Dougherty R H, Skolasky R L, Jr., McArthur J C: Progression of HIV-associated dementia treated with HAART. AIDS Read 2002, 12: 69-74.
Dow-Edwards, D. (1990) Fetal and maternal cocaine levels peak rapidly following intragastric administration in the rat. J. Subst. Abuse, 2:427-437.
Downs, A W, Eddy, N B (1932) The effect of repeated doses of cocaine on the rat. J. Pharmacol-Exp Ther 46:199-200.
Draper, N. R. and Smith, H. (1981) Applied Regression Analysis, 2nd ed., John Wiley & Sons: New York.
Dudish S A, Hatsukami D K (1996) Gender differences in crack users who are research volunteers. Drug Alcohol Depend 42(1):55-63.
Emmett-Olglesby, M W (1995) Sensitization and tolerance to motivational and subjective effects of psychostimulants. In: The neurobiology of cocaine: Cellular and molecular mechanisms, (Hammer, R P, Jr., ed), pp 31-47 Boca Raton; CRC Press.
Epstein L G, Sharer L R, & Goudsmit J. (1988). Neurological and neuropathological features of human immunodeficiency virus infection in children. Annals of Neurology, 23(suppl), S19-S23.
Epstein L G, Sharer L R, Oleske J M, Connor E M, Goudsmit J, Bagdon L, Robert-Guroff M, & Koenigsberger M R. (1986). Neurologic manifestations of human immunodeficiency virus infection in children. Pediatrics, 78, 678-687.
Epstein L G. (1998) HIV neuropathogenesis and therapeutic strategies. Acta Paediatr Jpn; 40: 107-111.
Epstein, L. G. (1998) HIV neuropathogenesis and therapeutic strategies. Acta Paediatr Jpn. 40, 107-11.
Evans S M, Cone E J, Henningfield J E (1996) Arterial and venous cocaine plasma concentrations in humans: relationship to route of administration, cardiovascular effects and subjective effects. J Pharmacol Exp Ther 279(3):1345-1356.
Evans, S. M., Cone, E. J. and Henningfield, J. E. (1996) Arterial and venous cocaine plasma concentration in humans: Relationship to route of administration, cardiovascular effects and subjective effects. J. Pharmacol. Exp. Ther. 279:1345-1356.
Fadel, J., Sarter, M. and Bruno, J. P. (2001) Basal forebrain glutamatergic modulation of cortical acetylcholine release. Synapse 39: 201-212.
Farzadegan J H, Hoover D R, Astemborski J, Lyles C M, Margolick J B, Markham R B et al.: Sex differences in HIV-1 viral load and progression to AIDS. Lancet 1998, 352: 1510-1514.
Fauci A S. (1988). The human immunodeficiency virus: Infectivity and mechanisms of pathogenesis. Science, 239(4840), 617-622.
Fauci, A. S. The human immunodeficiency virus: Infectivity and mechanisms of pathogenesis, Science 239 (1988) 617-622.
Fein, G., Biggins, C. A., MacKay, S. Delayed latency of the event-related brain potential P3A component in HIV disease, Arch Neurol 52 (1995) 1109-1118.
Fendt M, Li L, & Yeomans J S (2001). Brain stem circuits mediating prepulse inhibition of the startle reflex, Psychopharmacology, 156, 216-224.
Floyd R A, Hensley K, Jaffery F, Maidt L, Robinson K, Pye Q, Stewart 0.1999. Increased oxidative stress brought on by pro-inflammatory cytokines in neurodegenerative processes and the protective role of nitrone-based free radical traps. Life Sci 65:1893-1899.
Fray P J, Sahakian B J, Robbins T W Koob G F, Iverson S D (1980) An observational method for quantifying the behavioral effects of dopamine agonists: Contrasting effects of d-amphetamine and apomorphine. Psychopharmacology 69: 253-259.
Fried G, Andersson E, Csöregh L, Enmark E, Gustafsson J-A, Aanesen A et al.: Estrogen receptor beta is expressed in human embryonic brain cells and is regulated by 17b-estradiol. Eur J Neurosci 2004, 20: 2345-2354.
Gajjar T, Anderson L, Dluzen D: Acute effects of estrogen upon methamphetamine induced neurotoxicity of the nigrostriatal dopaminergic system. J Neural Transm 2003, 110: 1215-1224.
Garcia-Segura L M, Azcoitia I, DonCarlos, L L. 2001. Neuroprotection by estradiol. Prog Neurobiol 63:29-60.
Gardiner S A, Morrison M F, Mozley P D, et al. (2004) Pilot study on the effect of estrogen replacement therapy on brain dopamine transporter availability in healthy, postmenopausal women. Am. J. Geriatr. Psychiatry. 12:621-630.
Garnier M, DiLorenzo D, Albertini A, Maggi A: Identification of estrogen-responsive genes in neuroblastoma SK-ER3 cells. J Neurosci 1997, 17: 4591-4599.
Gavriil E S, Cooney R, Weeks BS. 2000. Tat mediates apoptosis in vivo in the rat central nervous system. Biochem Biophys Res Comm 267:252-256.
Gelbard H A, Nottet H S, Swindells S, Jett M, Dzenko K A, Genis P et al.: Platelet-activating factor: a candidate human immunodeficiency virus type 1-induced neurotoxin. J Virol 1994, 68: 4628-4635.
Giacobini, G (1998) Aging, Alzheimer's disease and estrogen therapy. Exp. Gerontol. 33:865-869.
Gillette J S, Bloomquist J R. (2003) Differential up-regulation of striatal dopamine transporter and α-synuclein by the pyrethroid insecticide permethrin. Toxicol. Appl. Pharmacol. 192:287-293.
Golden G, Mason P, Rubin R, Mason R: Biophysical membrane interactions of steroid hormones: A potential complementary mechanism of steroid action. Clinical Neuropharmacology 1998, 21: 181-189.
Goodkin, K., Shapshak, P., Metsch, L. R., McCoy, C. B., Crandall, K. A., Kumar, M., Fujimura, R. K., McCoy, V., Zhang, B. T., Reyblat, S., Xin, K. Q., Kumar, A. M. (1998) Cocaine abuse and HIV-1 infection: epidemiology and neuropathogenesis. J. Neuroimmunol. 83, 88-101.
Goodman J. H., Sloviter R. S. (1993) Cocaine neurotoxicity and altered neuropeptide Y immunoreactivity in the rat hippocampus; a silver degeneration and immunocytochemical study. Brain Res. 616, 263-72.
Goodwin, G. M., Pretsell, D. O., Chiswick, A., Egan, V., Brettle, R. P. The Edinburgh cohort of HIV-positive injecting drug users at 10 years after infection: a case-control study of the evolution of dementia, AIDS 10 (1996) 431-440.
Green P, Bishop J, Simpkins J W: 17a-Estradiol exerts neuroprotective effects on SK-N-SH cells. J Neurosci 1997, 17: 511-515.
Green P S, Gordon K, Simpkins J W. 1997. Phenolic A ring requirement for the neuroprotective effects of steroids. J Steroid Biochem Mol Biol 63:229-235.
Green P S, Gridley K E, Simpkins J W. 1996. Estradiol protects against □-amyloid (25-35)-induced toxicity in SK-N-SH human neuroblastoma cells. Neurosci Lett 218:165-168.
Green P S, Simpkins J W. 2000. Neuroprotective effects of estrogens: potential mechanism of action. Int J Devl Neurosci 18:347-358.
Green P S, Yang S H, Nilsson K R, Kumar A S, Covey D F, Simpkins J W. 2001. The nonfeminizing enantiomer of 17□-estradiol exerts protective effects in neuronal cultures and a rat model of cerebral ischemia. Endocinol 142:400-406.
Greenhouse, S. W. and Geisser, S. (1959) On methods in the analysis of profile data. Psychometrika, 24:95-112.
Grese, T. A. and Dodge, J. A. (1998) Selective estrogen receptor modulators (SERMs). Curr. Pharm. Des. 4:71-92.
Gridley K E, Green P S, Simpkins J W. 1997. Low concentrations of estradiol reduce □-amyloid (25-35)-induced toxicity, lipid peroxidation and glucose utilization in human SK-N-SH neuroblastoma cells. Brain Res 778:158-165.
Gridley K B, Green P S, Simpkins J W. 1998. A novel, synergistic interaction between 17□-estradiol and glutathione in the protection of neurons against □-amyloid 25-35-induced toxicity in vitro. Mol Pharmacol 54:874-880.
Gu J, Yassini P, Goldberg G, Shu W, Konat G, Wiggins R: Cocaine cytotoxicity in serum-free environment: C6 glioma cell culture. Neurotoxicology 1993, 14: 19-22.
Gurwell J A, Nath A, Sun Q, Zhang J, Martin K M, Chen Y, Hauser K F. 2001. Synergistic neurotoxicity of opioids and human immunodeficiency virus-1 tat protein in striatal neurons in vitro. Neurosci 102:555-563.
Hammond J, Quynh L, Goodyer C, Gelfand M, Trifiro M, LeBlanc A: Testosterone-mediated neuroprotection through the androgen receptor in human primary neurons. J Neurochem 2001, 77: 1319-1326.
Harrod S B, Booze R M, Welch M, Browning C E, Mactutus C F (2005) Acute and repeated intravenous cocaine-induced locomotor activity is altered as a function of sex and gonadectomy in rats. Pharmacol Biochem Behav 82(1):170-181.
Harrod S B, Mactutus C F, Beimett K, Hasselrot U, Wu G, Welch M, Booze R M (2004) Sex differences and repeated intravenous nicotine: behavioral sensitization and dopamine receptors. Pharmacol Biochem Behav 78(3):581-592.
Harrod S B, Mactutus C F, Browning C E, Welch M, Booze R M (2005) Homecage observations following acute and repeated IV cocaine in intact and gonadectomized rats. Neurotoxicol Teratol 27(6):891-896.
Harrod, S. B., Mactutus, C. F., Welch, M., Browning, C. E., Booze, R. M. (submitted-in Appendix) Acute and repeated intravenous cocaine-induced locomotor activity is altered as a function of sex and gonadectomy.
Haughey N J, Holden C P, Nath A, Geiger J D. 1999. Involvement of inositol 1,4,5-triphosphate-regulated stores of intracellular calcium in calcium dysregulation and neuron cell death caused by HIV-1 protein Tat. J Neurochem 73:1363-1374.
Haughey, N. J., Cutler, R. G., Tamara, A., McArthur, J. C., Vargas, D. L., Pardo, C. A., Turchan, J., Nath, A., Mattson, M. P. (2004) Perturbation of sphingolipid metabolism and ceramide production in HIV-dementia. Ann Neurol. 55, 257-267.
Haughey, N. J., Mattson, M. P. (2002) Calcium dysregulation and neuronal apoptosis by the HIV-1 proteins Tat and gp120. J Acquir Immune Defic Syndr. 31, Suppl 2, S55-S61.
Hawkins V, Shen Q, Chiueh C C. 1999. Kynostatin and 17□-estradiol prevent the apoptotic death of human neuroblastoma cells exposed to HIV-1 protease. Biomed Sci 6:433-438.
Hill J M, Mervis R F, Avidor R, Moody T W, Brenneman D E (1993). HIV envelope protein-induced neuronal damage and retardation of behavioral development in rat neonates, Maragos W F, Tillman P, Jones M, Bruce-Keller A J, Roth S, Bell J E, Nath A. (2003). Neuronal injury in hippocampus with human immunodeficiency virus transactivating protein, Tat. Neuroscience, 117:43-53.
Hill, J M, Mervis, R F., Avidor, R., Moody, T W, & Brenneman, D E. (1993). HIV envelope protein-induced neuronal damage and retardation of behavioral development in rat neonates, Brain Research, 603, 222-233.
Hoffman H S, & Ison J R (1980). Reflex modification in the domain of startle: Some empirical findings and their implications for how the nervous system processes sensory input. Psychological Review, 87(2), 175-189.
Hoffman, F. M., Dohadwala, M. M., Wright, A. D., Hinton, D. R., and Walker, S. M. (1994). Exogenous tat protein activates central nervous system-derived endothelial cells. J Neuroimmunol 54: 19-28.
Hojo Y, Hattori T, Enami T, Furukawa A, Suzuki K, Ishii H et al.: Adult male rat hippocampus synthesizes estradiol from pregnenolone by cytochromes P45017a and P450 aromatase localized in neurons. Proc Natl Acad Sci USA 2003, PNAS Early Edition: 1-6.
Holman B, Garada B, Johnson K, Mendleson J, Hallgring E, Teoh S et al.: A comparison of brain perfusion SPECT in cocaine abuse and AIDS dementia complex. J Nucl Med 1992, 33: 1312-1315.
Holmberg, S. D. (1996) The estimated prevalence and incidence of HIV in 96 large US metropolitan areas. Am. J. Public Health 86:642-654.
Holzbauer, M. and Youdim, M. B. H. (1973) The oestrous cycle and monoamine oxidase activity. Br. J. Pharmacol. 48:600-608.
Horger B A, Giles M K, Schenk S (1992) Preexposure to amphetamine and nicotine predisposes rats to self-administer a low dose of cocaine. Psychopharmacology 107(2-3):271-6.
Howard S A, Brooke S M, Sapolsky R M. 2001. Mechanism of estrogenic protection against gp120-induced neurotoxicity. Exp Neurol 168:385-391.
Huang J, Guan H, Booze R M, Eckman C B, Hersh L B (2004) Estrogen regulates neprilysin activity in rat brain. Neurosci. Lett. 367(1): 85-7.
Hudson L, Liu J, Nath A, Jones M, Raghavan R, Narayan O et al. (2000) Detection of the human immunodeficiency virus regulatory protein tat in CNS tissues. J Neurovirol; 6: 145-155.
Hudson L, Liu J, Nath A, Jones M, Raghavan R, Narayan O, Male D, Everall I (2000) Detection of the human immunodeficiency virus regulatory protein tat in CNS tissues. Neurovirol 6:145-155.
Hudson, L., Liu, J., Nath, A., Jones, M., Raghavan, R., Narayan, O., Male, D., Everall, I. (2000) Detection of the human immunodeficiency virus regulatory protein tat in CNS tissues. J. Neurovirol. 6, 145-155.
Ickovics, J and Rodin, J. (1992) Women and AIDS in the United States: Epidemiology, natural history and mediating mechanism. Health Psychology, 11: 1-16.
Imperato A and DiChiara G (1984) Trans-striatal dialysis coupled to reverse-phase high performance liquid chromatography with electrochemical detection: A new method for study of the in vivo release of endogenous dopamine and metabolites. J. Pharmacol. Exp. Ther. 4:966-977.
Itoh K., Mehraein P., Weis S. (2000) Neuronal damage of the substantia nigra in HIV-1 infected brains. Acta Neuropathol (Berl) 99: 376-384.
Jain S K, Kannan K, Prouty L, Jain S K. 2004. Progesterone, but not 17□-estradiol, increases TNF-□ secretion in U937 monocytes. Cytokine 26:102-105.
Jiang N, Chopp M, Stein D, Feit H: Progesterone is neuroprotective after transient middle cerebral artery occlusion in male rats. Brain Res 1996, 735: 101-107.
Jones M, Olafson K, Del Bigio M R, Peeling J, Nath A. 1998. Intraventricular injection of human immunodeficiency virus type 1 (HIV-1) tat protein causes inflammation, gliois, apoptosis, and ventricular enlargement. J Neuropathol Exp Neurol 57:563-570.
Jones M V, Bell J E, Nath A. (2000) Immunolocalization of HIV envelope gp120 in HIV encephalitis with dementia. AIDS; 14: 2709-2713.
Jones, M. V., Bell, J. E., Nath, A. (2000) Immunolocalization of HIV envelope gp120 in HIV encephalitis with dementia. AIDS. 14, 2709-2713.
Jori, A., Colturani, F., Dolfini, E., and Rutczynski, M. (1976) Modifications of the striatal dopamine metabolism during the estrus cycle in mice. Neuroendocrinology, 21:262:266.
Jovanovic T, Szilagyi S, Chakravorty S, Fiallos A M, Lewison B J, Parwani A, M P Schwartz, Gonzenbach S, Rotrosen J P, & Duncan E J. (2004). Menstrual cycle phase effects on prepulse inhibition of acoustic startle, Psychophysiology, 41, 401-406.
Jover T, Tanaka H, Calderone A, Oguro K, Bennett M, Etgen A et al.: Estrogen protects against global ischemia-induced neuronal death and prevents activation of apoptotic signaling cascades in the hippocampal CA1. J Neurosci 2002, 22: 2115-2124.
Joyce E M, Koob G F (1981) Amphetamine-, scopolamine- and caffeine-induced locomotor activity following 6-hydroxydopamine lesions of the mesolimbic dopamine system. Psychopharmacology 73(4):311-313.
Kalivas P W (2004) Recent understanding in the mechanisms of addiction. Curr Psychiatry Rep 6(5):347-351.
Kalivas P W, Volkow N D (2005) The neural basis of addiction: a pathology of motivation and choice. Am J Psychiatry 162(8):1403-1413.
Kalivas P W, Weber B (1988) Amphetamine injection into the ventral mesencephalon sensitizes rats to peripheral amphetamine and cocaine. J Pharmacol Exp Ther 245(3):1095-1102.
Kalivas, P. W. (1995) Neural basis for sensitization to cocaine. In: The Neurobiology of cocaine: Cellular and molecular mechanisms. R. P. Hammer, Jr. Ed, CRC Press, Boca Raton, pp 81-98.
Kawas C, Resnick S, Morrison A, Brookmeyer R, Corrada M, Zonderman A et al.: A prospective study of estrogen replacement therapy and the risk of developing Alzheimer's disease: the Baltimore Longitudinal Study of Aging. Neurology 1997, 48: 1517-1521.
Kendall S L, Anderson C F, Nath A, Turchan-Cholewo J, Land C L, Mactutus C F, Booze R M (2005) Gonadal steroids differentially modulate neurotoxicity of HIV and cocaine: Testosterone and ICI 182,780 sensitive mechanisms. BMC Neuroscience 8; 6(1):40.
Kendall, S. L., Anderson, C., Nath, A., Mactusus, C. F., Booze, R. M. Gonadal steroids' effects of synergistic neurotoxicity of cocaine with HIV proteins. College on Problems of Drug Dependence. Quebec City, Quebec, 2002.
Kibayashi, K., Mastri, A. R., Hirsch, C. S. (1996) Neuropathology of human immunodeficiency virus infection at different disease stages. Hum Pathol. 27, 637-642.
Koch M, & Schnitzler H-U. (1997). The acoustic startle response in rats—circuits mediating evocation, inhibition and potentiation. Behavioral Brain Research. 89(1-2), 35-49.
Koob G F (1992) Neural mechanisms of drug reinforcement. Ann N Y Acad Sci 654: 171-191.
Koob G F, Stinus L, Le Moal M (1981) Hyperactivity and hypoactivity produced by lesions to the mesolimbic dopamine system. Behav Brain Res 3(3):341-359.
Korneyev A, Pan B, Plol A, Romeo E, Guidotti A, Costa E: Stimulation of brain pregnenolone synthesis by mitochondrial diazepam binding inhibitor receptor ligands in vivo. J Neurochem 1993, 61: 1515-1524.
Kosten T A, Gawin F H, Kosten T R, Rounsaville B J (1993) Gender differences in cocaine use and treatment response. J Subst Abuse Treat 10(1):63-66.
Koutsilieri E, Sopper S, Scheller C, ter Meulen V, Riederer P (2002) Involvement of dopamine in the progression of AIDS Dementia Complex. J Neural Transm 109(3):399-410.
Koutsilieri, E, Gotz, M. E, Sopper, S, Sauer, U, Demuth, M, ter Meulen, V, Riederer, P. (1997) Regulation of glutathione and cell toxicity following exposure to neurotropic substances and human immunodeficiency virus-1 in vitro. J. Neurovirol. 3, 342-349.
Koutsilieri, E., Sopper, S., Scheller, C., ter Meulen, V., Riederer, P. (2002) Parkinsonism in HIV dementia. J. Neural Transm. 109(5-6): 767-75. Review.
Koutsiliery E., Sopper S., Scheller C., V. ter Meulen, Riederer P. (2002) Involvement of dopamine in the progression of AIDS dementia complex. J. of Neural Transm 109: 399-410.
Kozlowski M R, Sandler P, Lin P F, Watson A. 1991. Brain-derived cells contain a specific binding site for gp120 which is not the CD4 antigen. Brain Res 553:300-304.
Kruman I I, Nath A, Mattson M P (1998) HIV-1 protein Tat induces apoptosis of hippocampal neurons by a mechanism involving caspase activation, calcium overload, and oxidative stress. Exp Neurol 54:276-288.
Kruman I I, Nath A, Mattson M P. 1998. HIV-1 protein Tat induces apoptosis of Hippocampal neurons by a mechanism involving caspase activation, calcium overload, and oxidative stress. Exp Neurol 154:276-288.
Kubos K L, Moran T H, Robinson R G (1987) Differential and asymmetrical behavioral effects of electrolytic or 6-hydroxydopamine lesions in the nucleus accumbens. Brain Res 13; 401(1):147-151.
Kumer S C, Vrana K E. (1996) Intricate regulation of tyrosine hydroxylase activity and gene expression. J Neurochem; 67: 443-462.
Labrie F, Belanger A, Cusan L, Candas B: Physiological changes in dehydroepiandrosterone are not reflected by serum levels of active androgens and estrogens but of their metabolites: Intracrinology. J Clin Endocrinol Metab 1997, 82: 2403-2409.
Lannuzel A, Barnier J V, Hery C, van tan H, Guibert B, Gray F, Vincent J D, Tardieu (1997). Human immunodeficiency virus type 1 and its coat protein gp120 induce apoptosis and activate JNK and ERK mitogen-activated protein kinases in human neurons. Annals of Neurology, 42(6):847-856.
Lee Y W, Eum S Y, Nath A, Toborek M (2004) Estrogen-mediated protection against HIV Tat protein-induced inflammatory pathways in human vascular endothelial cells. Cardiovasc Res.; 63(1):139-48.
Lee Y W, Eum S Y, Nath A, Toborek M. 2004. Estrogen-mediated protection against HIV Tat protein-induced inflammatory pathways in human vascular endothelial cells. Cardiovasc Res 63:139-148.
Lee, F. J. S., Liu, F., Pristupa, Z. B., Niznik, H. B. (2001) Direct binding and functional coupling of α-synuclein to the dopamine transporters accelerate dopamine-induced apoptosis. FASEB J. 15:916-926.
Lee, K. H., Kim, M. Y., Kim, D. H., Lee, Y. S. (2004) Syntaxin 1A and receptor for activated C kinase interact with the N-terminal region of human dopamine transporter. Neurochem. Res. 29:1405-1409.
Levesque, D. and D1 Paolo, T. (1988) Rapid conversion of high into low striatal D2-dopamine receptor agonist binding states after an acute physiological dose of beta-estradiol. Neurosci. Letters 88(1):113-118.
Levine, R. L., Garland, D., Oliver, C. N., Amici, A., Climent, I., Lenz, A.-G. et al. (1990) Determination of protein carbonyl content in oxidatively modified proteins. Methods Enzymol. 233:346-357.
Li L-B, Chen N, Ramamoorthy S, Chi L, Cui X-N, Wang L C et al. (2004) The role of N-glycosylation in function and surface trafficking of the human dopamine transporter. The Journal of Biological Chemistry; 279: 21012-21020.
Li L B, Chen N, Ramamoorthy S, Chi L, Cui X N, Wang L C, Reith M E A. (2004) The role of N-glycosylation in function and surface trafficking of the human dopamine transporter. J. Biol. Chem. 279:21012-21020.
Lipton S A. 1998. Neuronal injury associated with HIV-1: approaches to treatment. Ann Rev Pharmacol Toxicol 38:159-177.
Loder, M. K., Melikian, H. E. (2003) The dopamine transporter constitutively internalizes and recycles in a protein kinase C-regulated manner in stably transfected PC 12 cell lines. J. Biol. Chem. 278:22168-22174.
Lunte, S. M. and Lunte, C. E. (1996) Microdialysis sampling from pharmacological studies: HPLC and CE analysis. Adv. Chromatogr. 36:383-432.
Lyons, K. E., Hubble, J. P., Troster, A. I., Pahwa, R. and Koller, W. C. (1998) Gender differences in Parkinson's disease. Clin. Neuropharmacol. 21:118-121.
Ma M, Nath A (1997) Molecular determinants for cellular uptake of Tat protein of human immunodeficiency virus type 1 in brain cells. J Virol 71(3):2495-2499.
Ma, M., Nath, A. (1997) Molecular determinants for cellular uptake of Tat protein of human immunodeficiency virus type 1 in brain cells. J. Virol. 71, 2495-2499.
Mabrouk, K., Van Rietschoten, J., Vives, E., Darbon, H., Rochat, H., Sabatier, J. M. (1991) Lethal neurotoxicity in mice of the basic domains of HIV and SIV Rev proteins. Study of these regions by circular dichroism. FEBS Lett. 289, 13-17.
Maccarrone M, Navarra M, Catani V, Corasaniti Mont., Bagetta G, Finazazi-Agrò A: Cholesterol-dependent modulation of the toxicity of HIV-1 coat protein gp120 in human neuroblastoma cells. J Neurochem 2002, 82: 1444-1452.
Mactutus, C. F., Harper, G., Booze, R. M. and Riccio, D. C. (1995) Gender and parity: Potential sources of variance in ACTH-induced memory reactivation in weanling rats. Psychobiol. 23:199-203.
Mactutus, C. F., Herman, A. S., and Booze, R. M. (1994) Chronic intravenous model for studies of drug (ab)use in the pregnant and/or group-housed rat: An initial study with cocaine. Neurotoxicol. Teratol., 16:183-191.
Mactutus, C F, Herman A S, Booze, R M (1994) Chronic intravenous model for studies of drug (ab)use in the pregnant and/or group-housed rat: An initial study with cocaine. Neurotoxicol Teratol 16:183-191.
Magnuson D S, Knudsen B E, Geiger J D, Brownstone R M, Nath A: Human immunodeficiency virus type 1 tat activates non-N-methyl-D-aspartate excitatory amino acid receptors and causes neurotoxicity. Ann Neurol 1995, 37: 373-380.
Manji H, Miller R. (2004) Neurology of HIV infection J. Neurol. Neurosurg. Psychiatry; 75: 29-35.
Manly J, Merchant C, Jacobs D, Small S, Bell K, Ferin M et al.: Endogenous estrogen levels and Alzheimer's disease among postmenopausal women. Neurology 2000, 54: 833-837.
Maragos W F, Tillman P, Jones M, Bruce-Keller A J, Roth S, Bell J E, & Nath A. (2003). Neuronal injury in hippocampus with human immunodeficiency virus transactivating protein, Tat. Neuroscience, 117, 43-53.
Margolin A, Avants S, Warburton L, Hawkins K: Factors affecting cognitive functioning in a sample of human immunodeficiency virus-positive injection drug users. AIDS Patient Care and STDs 2002, 16: 255-267.
Martinez, Z. A., Halim, N. D., Oostwegel, J. L., Geyer, M. A., Swerdlow, N. R. Ontogeny of phencyclidine and apomorphine-induced startle gating deficits in rats, Pharmacol Biochem Behav 65 (2000) 449-457.
Mash D C, Pablo J, Ouyang Q, Hearn W L, Izenwasser S. (2002) Dopamine transport function is elevated in cocaine users. J Neurochem; 81:292-300.
McCullough L, Blizzard K, Simpson E, Ohran K, Hurn P: Aromatase cytochrome P450 and extragonadal estrogen play a role in ischemic neuroprotection. J Neurosci 2003, 23: 8701-8705.
Melikian H E, Buckley K M. (1999) Membrane trafficking regulates the activity of the human dopamine transporter. J. Neurosci. 19:7699-7710.
Merchenthaler I, Dellovade T, Shugrue P: Neuroprotection by estrogen in animal models of global and focal ischemia. Ann N Y Acad Sci 2003, 1007: 89-100.
Messam C A, Hou J, Berman J W, Major E O: Analysis of the temporal expression of nestin in human fetal brain derived neuronal and glial progenitor cells. Developmental Brain Research 2002, 134: 87-92.
Meucci O, Miller R J. 1996. gp120-induced neurotoxicity in Hippocampal pyramidal neuron cultures: protective action of TGF-□1. J Neurosci 16:4080-4088.
Miller G W, Staley J K, Heilman C J, Perez J T, Mash D C, Rye D B et al. (1997) Immunochemical analysis of dopamine transporter protein in Parkinson's Disease. Ann. Neurology; 41: 530-539.
Mingjia L, Short R. 2002. How oestrogen or progesterone might change a woman's susceptibility to HIV-1 infection. Aust N Z J Obstet Gynaecol 42:472-475.
Mirsattari, S. M., Power, C., and Nath, A. (1998). Parkinsonism with HIV infection. Mov Disord 13: 684-9.
Moore, H., Fadel, J., Sarter, M. and Bruno, J. P. (1999) Interactions between dopamine and benzodiazepine receptors in the modulation of cortical acetylcholine release. Neuroscience 88: 811-822.
Moosmann B, Behl C: The antioxidant neuroprotective effects of estrogens and phenolic compounds are independent from their estrogenic properties. Proc Natl Acad Sci U S A 1999, 96: 8867-8872.
Morishima, H. O., Matuso, A. Y., Akiba, K., Masaoka, T. and Cooper, T. B. (1993) Gender-related differences in cocaine toxicity in the rat. J. Lab. Clin. Med. 122:128-129:
Moron J A, Zakharova I, Ferrer J V, et al. (2003) Mitogen-activated protein kinase regulates dopamine transporter surface expression and dopamine transport capacity. J. Neurosci. 23:8480-8488.
Mortensen O V, Amara S G. (2003) Dynamic regulation of the dopamine transporter. Eur. J. Pharmacol. 479:159-170.
Moss R, Gu J, Wong M: Estrogen: Nontranscriptional signaling pathway. In Recent Progress in Hormone Research. The Endocrine Society; 1997:33-69.
Murphy S J, Traystman R J, Hum PD. 2000. Progesterone exacerbated striatal stroke injury in progesterone-deficient female animals. Stroke 31:1173-1178.
Murray, A. M., Ryoo, H., and Joyce, J. N. (1992) Visualization of dopamine D3-like receptors in human brain with [125I]epidepride. Eur. J. Pharmacol., 227:443-445.
Muscoli C, Salvemini D, Paolino D, Iannone M, Palma E, Cufari A, Rotiroti D, Pemo C F, Aquaro S, Mollace V. 2002. Peroxynitrite decomposition catalyst prevents apoptotic cell death in a human astrocytoma cell line incubated with supernatants of HIV-infected macrophages. BMC Neuroscience 3:13.
Nassogne M, Evrard P, Courtoy P: Selective neuronal toxicity of cocaine in embryonic mouse brain cocultures. Proc Natl Acad Sci USA 1995, 92: 11029-11033.
Nassogne M, Louahed J, Evrard P, Courtoy P: Cocaine induced apoptosis in cortical neurons in fetal mice. J Neurochem 1997, 68: 2442-2450.
Nath A (2002) Human immunodeficiency virus (HIV) proteins in neuropathogenesis of HIV dementia. J Infect Dis 1; 186 Suppl 2:S193-8.
Nath A, Anderson C, Jones M, Maragos W, Booze R, Mactutus C et al. (2000) Neurotoxicity and dysfunction of dopaminergic systems associated with AIDS dementia. Journal of Psychopharmacology; 14: 222-227.
Nath A, Anderson C, Jones M, Maragos W, Booze R, Mactutus C F, Bell J, Hauser K F, & Mattson M. (2000). Neurotoxicity and dysfunction of dopaminergic systems associated with AIDS dementia. Journal of Psychopharmacology, 14(3), 222-227.
Nath A, Geiger J D, Mattson M P, Magnuson D S K, Jones M, Berger JR. 1998.: Role of viral proteins in HIV-1 neuropathogenesis with emphasis on Tat. NeuroAIDS 1:6.
Nath A, Haughey N J, Jones M, Anderson C, Bell J E, Geiger J D. 2000.: Synergistic neurotoxicity by human immunodeficiency virus proteins Tat and gp120: protection by memantine. Ann Neurol 47:186-194.
Nath A, Hauser K F, Wojna V, Booze R M, Maragos W, Prendergast M et al. (2002) Molecular basis for interactions of HIV and drugs of abuse. J. Acquir. Immune Defic. Syndr; 31:S62-S69.
Nath A, Maragos W F, Avison M J, Schmitt F A, Berger J R (2001) Acceleration of HIV dementia with methamphetamine and cocaine. J Neurovirol 7:66-71.
Nath A, Psooy K, Martin C, knudsen B, Magnuson D S K, Haughey N, Geiger J D. 1996. Identification of a human immunodeficiency virus type 1 Tat epitope that is neuroexcitatory and neurotoxic. J Virol 70:1475-1480.
Nath, A., Anderson, C., Jones, M., Maragos, W. F., Booze, R. M., Mactutus, C. F., Bell, J., Hauser, K. F., Mattson, M. Neurotoxicity and dysfunction of dopaminergic systems associated with AIDS dementia, J Psychopharmacol 14 (2000) 222-227.
Nath, A., Geiger, J. (1998) Neurobiological aspects of human immunodeficiency virus infection: neurotoxic mechanisms. Prog Neurobiol. 54, 19-33.
Nath, A., Hauser K. F., Wojna V., Booze R. M., Maragos W., Prendergast M., Cass W., Turchan J. T. (2002) Molecular basis for interactions of HIV and drugs of abuse. J. Acquir. Immune Defic. Syndr. 31 Suppl 2, S62-S69. Review.
Nath, A., Maragos, W. F., Avison, M. J., Schmitt, F. A., Berger, J. R. (2001) Acceleration of HIV dementia with methamphetamine and cocaine. J. Neurovirol. 7, 66-71.
Navia B. A., Cho E. S., Petito C. K., Price R. W. (1986) The AIDS dementia complex. II. Neuropathology. Ann. Neurol. 19: 525-535.
Nestler E J (2001) Neurobiology. Total recall-the memory of addiction. Science 292:2266-2267.
Neuenburg, J. K., Brodt, H. R., Hemdier, B. G., Bickel, M., Bacchetti, P., Price, R. W., Grant, R. M., Schlote, W. J (2002) HIV-related neuropathology, 1985 to 1999: rising prevalence of HIV encephalopathy in the era of highly active antiretroviral therapy. Acquir Immune Defic Syndr. 31, 171-177.
New D, Ma M, Epstein L, Nath A, Gelbard H: Human immunodeficiency virus type 1 Tat protein induces death by apoptosis in primary human neuron cultures. J Neurovirol 1997, 3: 168-173.
New D R, Maggirwar S B, Epstein L G, Dewhurst S, Gelbard H A. 1998. HIV-1 Tat induces neuronal death via tumor necrosis factor-□ and activation of non-N-methyl-D-aspartate receptors by a NF□B-independent mechanism. J Biol Chem 273:17852-17858.
Okamoto M, Wang X, Debyser Z, De Clerq E, Baba M. 2003. Establishment of in vitro assay system mimicking human immunodeficiency virus type 1-induced neural cell death and evaluation of inhibitors thereof. J Virol Meth 108:195-203.
Papadopoulos V, Guarneri P, Krueger K, Guidotti A: Pregnenolone biosynthesis in C6-2B glioma cell mitochondria: Regulation by a mitochondrial diazepam binding inhibitor receptor. Proc Natl Acad Sci USA 1992, 89: 5113-5117.
Paquette B, Cantin A M, Kocsis-Bedard S, Barry S, Lemay R, Jay-Gerin J P. 2001. In vitro generation of peroxynitrite by 2- and 4-hydroxyestrogens in the presence of nitric oxide. Chem Res Toxicol 14:547-554.
Parsons, L. H. and Justice, J. B., Jr. (1994) Quantitative approaches to in vivo brain microdialysis. Crit. Rev. Neurobiol. 8:189-220.
Paxinos, G. & Watson, C. The Rat Brain in Stereotaxic Coordinates. 2nd Ed., Academic Press, New York, 1986.
Perez, A., Probert, A. W., Wang, K. K., Sharmeen, L. (2001) Evaluation of HIV-1 Tat induced neurotoxicity in rat cortical cell culture. J. Neurovirol. 7(1):1-10.
Peris J, Decambre N, Coleman-Hardee M L, Simpkins J W (1991) Estradiol enhances behavioral sensitization to cocaine and amphetamine-stimulated striatal [3H]dopamine release. Brain Res 566(1-2):255-64.
Perl, A., Banki, K. (2000) Genetic and metabolic control of the mitochondrial transmembrane potential and reactive oxygen intermediate production in HIV disease. Antioxid Redox Signal. 2, 551-573.
Phillipon, V., Vellutini, C., Gambarelli, D., Harkiss, G., Arbuthnott, G., Metzger, D., Roubin, R., Filippi, P. (1994) The basic domain of the lentiviral Tat protein is responsible for damages in mouse brain: Involvement of cytokines. Virology 205, 519-529.
Piazza P V, Deminiere J M, le Moal M, Simon H (1990) Stress- and pharmacologically-induced behavioral sensitization increases vulnerability to acquisition of amphetamine self-administration. Brain Res 23; 514(1):22-6.
Picazo O, Azoitia I, Garcia-Segura L M. 2003. Neuroprotective and neurotoxic effects of estrogen. Brain Res 990:20-27.
Pierce R C, Kalivas P W (1997) A circuitry model of the expression of behavioral sensitization to amphetamine-like psychostimulants. Brain Res Brain Res Rev 25(2):192-216.
Pike D J: Estrogen modulates neuronal Bcl-xl expression and beta-amyloid induced apoptosis relevance to Alzheimer's disease. J Neurochem 1999, 72: 1552-1563.
Polazzi E, Giulio L, Minghetti L. 1999. Human immunodeficiency virus type 1 Tat protein stimulates inducible nitric oxide synthase expression and nitric oxide production in microglial cultures. J Neuropathol Exp Neurol 58:825-831.
Post R M (1980) Intermittent versus continuous stimulation: Effect of time interval on the development of sensitization or tolerance. Life Sci 26:1275-1282.
Post R M, Contel N R (1983) Human and animal studies of cocaine: Implications for the development of behavioral pathology. In: Stimulants: Neurochemical, behavioral and clinical perspectives, (Creese I, ed), pp 169-203 New York, Raven Press.
Prendergast M A, Rogers D T, Mulholland P J, Littleton J M, Wilkins L H Jr, Self R L, Nath A (2002) Neurotoxic effects of the human immunodeficiency virus type-1 transcription factor Tat require function of a polyamine sensitive-site on the N-methyl-D-aspartate receptor. Brain Res 8; 954(2):300-307.
Prendergast M A, Rogers D T, Mulholland P J, Littleton J M, Wilkins L H, Self R L, Nath A. 2002. Neurotoxic effects of the human immunodeficiency virus type-1 transcription factor Tat require a function of a polyamine sensitive-site on the N-methyl-D-aspartate receptor. Brain Res 954:300-307.
Price R W, Brew B, Sidtis J, Rosenblum M, Scheck AC, & Cleary P. (1988). The brain in AIDS: Central nervous system HIV-1 infection and AIDS complex. Science, 239(4840) 586-592.
Price R W, Brew B J (1988). The AIDS dementia complex. The Journal of Infectious Diseases, 158(5):1079-1083.
Pristupa, Z. B., McConkey, F., Liu, F., Man, H. Y., Lee, F. J. S., Wang, Y. T., Niznik, H. B. (1998) Protein kinase-mediated bidirectional trafficking and functional regulation of the human dopamine transporter. Synapse. 30:79-87.
Puttfarcken, P. S., Manelli, A. M., Neilly, J., Frail D. E. (1996) Inhibition of age-induced □-amyloid neurotoxicity in rat hippocampal cells. Exp. Neurol. 138, 73-81.
Raber J, Toggas S M, Lee S, Bloom F E, Epstein C J, Mucke L. 1996. Central nervous system expression of HIV-1 gp120 activates the hypothalamic-pituitary-adrenal axis: evidence for involvement of the NMDA receptors and nitric oxide synthase. Virology 226:362-373.
Ratka, A. and Simpkins, J. W. (1990) Dose-dependent effects of chronic treatment with estradiol or progesterone on LH secretion in ovariectomized rats. Endocrine Res. Commun. 16:165-184.
Rau S, Dubal D, Böttner M, Gerhold L, Wise P: Estradiol attenuates programmed cell death after stroke-like injury. J Neurosci 2003, 23: 11420-11426.
Reyes, M. G., Faraldi, F., Senseng, C. S., Flowers, C., Fariello, R. (1991) Nigral degeneration in acquired immune deficiency syndrome (AIDS). Acta Neuropathol (Berl). 82(1):39-44.
Robbins T W, Roberts D C, Koob G F (1983) Effects of d-amphetamine and apomorphine upon operant behavior and schedule-induced licking in rats with 6-hydroxydopamine-induced lesions of the nucleus accumbens. J Pharmacol Exp Ther 224(3):662-673.
Robbins, T W, Cador, M, Taylor, J R, Everitt, B J (1989) Limbic-striatal interactions in reward-related processes. Neurosci Biobehav Rev 13:155-162.
Robinson S, Enters E, Jackson G, Chinchilli V, Maher J, McDowell K et al.: Maternal and fetal brain and plasma levels of cocaine and benzoylecgonine after acute or chronic maternal intravenous administration of cocaine. J Pharmacol Exp Ther 1994, 271: 1234-1239.
Robinson T E and Whishaw I Q (1988) Normalization of extracellular dopamine in striatum following recovery from a partial unilateral 6-OHDA lesion of the substantia nigra: A microdialysis study in a freely moving rat. Brain Res. 450:209-224.
Robinson T E, Berridge K C (1993) The neural basis of drug craving: an incentive-sensitization theory of addiction. Brain Res Brain Res Rev 18:247-291.
Rupprecht R, Friess E, Holsboer F: The Neuropsychopharmacological Potential of Neurosteroids. In Neurosteroids: A new regulatory function in the nervous system. Edited by Baulieu E-E, Robel P, Schumacher M. Totowa, N. J.: Humana Press; 1999:349-364.
Sabatier J. M., Vives E., Mabrouk K., Benjouad A., Rochat H., Duval A., Hue B., Bahraoui E. (1991) Evidence for neurotoxic activity of tat from human immunodeficiency virus type 1. J. Virol. 65, 961-967.
Saito Y, Sharer L R, Epstein L G, Michaels J, Mintz M, Louder M et al. (1994) Overexpression of nef as a marker for restricted HIV-1 infection of astrocytes in postmortem pediatric central nervous tissues. Neurology; 44: 474-481.
Saito, Y., Sharer, L. R., Epstein, L. G., Michaels, J., Mintz, M., Louder, M., Golding, K., Cvetkovich, T. A., Blumberg, B. M. (1994) Overexpression of nef as a marker for restricted HIV-1 infection of astrocytes in postmortem pediatric central nervous tissues. Neurology. 44, 474-481.
Samaha A N, Li Y, Robinson T E (2002) The rate of intravenous cocaine administration determines susceptibility to sensitization. J Neurosci 22(8):3244-3250.
Sanfeliu C, Sebastiá J, Kim S U: Methylmercury neurotoxicity in cultures of human neurons, astrocytes, neuroblastoma cells. Neurotoxicology 2001, 22: 317-327.
Saunders-Pullman R, Gordon-Elliott J, Parides M, Fahn S, Saunders H R, Bressman S: The effect of estrogen replacement on early Parkinson's disease. Neurology 1999, 52: 1417-1421.
Sawada H, Ibi M, Kihara T, Urushitani M, Honda K, Nakanishi M et al.: Mechanisms of antiapoptotic effects of estrogens in nigral dopaminergic neurons. FASEB J 2000, 14: 1202-1214.
Schneider LS. 2004. Estrogen and dementia: insights from the Woman's Health Initiative Memory Study. JAMA 291:3005-3007.
Schroeder, M. M., Handelsman, L., Torres, L., Jacobson, J., Ritter, W. Consistency of repeated event-related potentials in clinically stable HIV-1 infected drug users, J Neuropsychiat Clin Neurosci 17 (1996) 418-423.
Self R L, Mulholland P J, Nath A, Harris B R, Prendergast M A (2004) The human immunodeficiency virus type-1 transcription factor Tat produces elevations in intracellular Ca2+ that require function of an N-methyl-D-aspartate receptor polyamine-sensitive site. Brain Res January 2; 995(1):39-45.
Self R L, Mulholland P J, Nath A, Harris B R, Prendergast M A. 2003. The human immunodeficiency virus type-1 transcription factor Tat produces elevations in intracellular Ca2+ that require function of an N-methyl-D-aspartate receptor polyamine-sensitive site. Brain Res 885:39-45.
Shapshak P, Crandall K A, Xin K Q, Goodkin K, Fujimura R K, Bradley W, McCoy C B, Nagano I, Yoshioka M, Petito C, Sun N C, Srivastava A K, Weatherby N, Stewart R, Delgado S, Matthews A, Douyon R, Okuda K, Yang J, Zhangl B T, Cao X R, Shatkovsky S, Fernandez J B, Shah S M, Perper J (1996) HIV-1 neuropathogenesis and abused drugs: current reviews, problems, and solutions. Adv Exp Med Biol 402:171-186.
Shapshak, P., Crandall, K. A., Xin, K. Q., Goodkin, K., Fujimura, R. K., Bradley, W., McCoy, C. B., Nagano, I., Yoshioka, M., Petito, C., Sun, N. C., Srivastava, A. K., Weatherby, N., Stewart, R., Delgado, S., Matthews, A., Douyon, R., Okuda, K., Yang, J., Zhangl, B. T., Cao, X. R., Shatkovsky, S., Fernandez, J. B., Shah, S. M., Perper, J. (1996) HIV-1 neuropathogenesis and abused drugs: current reviews, problems, and solutions. Adv Exp Med. Biol. 402, 171-186.
Shi B, Raina J, Lorenzo A, Busciglio J, Gabuzda D (1998) Neuronal apoptosis induced by HIV-1 Tat protein and TNF-alpha: potentiation of neurotoxicity mediated by oxidative stress and implications for HIV-1 dementia. J Neurovirol 4(3):281-290.
Sidhu A, Wersinger C, Vernier P. (2004) α-Synuclein regulation of the dopaminergic transporter: a possible role in the pathogenesis of Parkinson's disease. FEBS Lett. 565:1-5.
Siegel S, Castellan N J Jr (1988) Nonparametric Statistics for the Behavioral Sciences, Second Edition, New York, N.Y.: McGraw-Hill, Inc.
Singer C, Rogers K, Dorsa D: Modulation of Bcl-2 expression: A potential component of estrogen protection in NT2 neurons. Neuroreport 1998, 9: 2565-2568.
Sircar R, Kim D (1999) Female gonadal hormones differentially modulate cocaine-induced behavioral sensitization in Fischer, Lewis, and Sprague-Dawley rats. J Pharmacol Exp Ther 289(1):54-65. Smith S M, Baskin G B, Marx P A. 2000. Estrogen protects against vaginal transmission of simian immunodeficiency virus. J Infect Dis 182:708-715.
Smith, M. A., Sayre, L. M., Anderson, V. E., Harris, P. L., Beal, M. F., Kowall, N., Perry, G. (1998) Cytochemical demonstration of oxidative damage in Alzheimer disease by immunochemical enhancement of the carbonyl reaction with 2,4-dinitrophenylhydrazine. J Histochem Cytochem. 46(6):731-5.
Song L, Nath A, Geiger J D, Moore A, Hoclunan S (2003) Human immunodeficiency virus type 1 Tat protein directly activates neuronal N-methyl-D-aspartate receptors at an allosteric zinc-sensitive site. J Neurovirol 9(3):399-403.
Song L, Nath A, Geiger J D, Moore A, Hochman S: Human immunodeficiency virus type 1 Tat protein directly activates neuronal N-methyl-D-aspartate receptors at an allosteric zinc-sensitive site. J Neurovirol 2003, 9: 399-403.
Sorkina T, Doolen S, Galperin E, Zahniser N R, Sorkin A. (2002) Oligomerization of dopamine transporters visualized in living cells by fluorescence resonance energy transfer microscopy. J. Biol. Chem. 278:28274-28283.
SPSS12.0 for Windows (1989-2003). SPSS Inc.
Squires K E: Treating HIV infection and AIDS in women. AIDS Read 2003, 13: 228-240.
Staley, J. K. and Mash, D. C. (1996) Adaptive increase in D3 dopamine receptors in the brain reward circuits of human cocaine fatalities. J. Neurosci. 16:6100-6106.
Sterling T, Vlahov D, Astermborski J, Hoover D, Margolick J, Quinn T: Initial plasma HIV-1 RNA levels and progression to AIDS in women and men. N Engl J Med 2001, 344: 720-725.
Stoffel-Wagner B: Neurosteroid metabolism in the human brain. Eur J Endocrinology 2001, 145: 669-679.
Strathdee, S. A., Galai, N., Safaiean, M., Celentano, D. D., Vlahov, D., Jolmson, L., Nelson, K. E. (2001) Sex differences in risk factors for hiv seroconversion among injection drug users: a 10-year perspective. Arch Intern Med. 161:1281-8.
Swerdlow N R, Braff D L, & Geyer M A. (2000). Animal models of deficient sensorimotor gating: what we know, what we think we know, and what we hope to know soon, Behavioural Pharmacology, 11, 185-204.
Swerdlow N R, Geyer M A, & Braff D L. (2001). Neural circuit regulation of prepulse inhibition of startle in the rat: current knowledge and future challenges, Psychopharmacology, 156, 194-215.
Swerdlow N R, Hanlon F M, Henning L, Kim Y K, Gaudet I, & Halim N D. (2001). Regulation of sensorimotor gating in rats by hippocampal NMDA: anatomical localization. Brain Research, 898, 195-203.
Swerdlow N R, Martinez Z A, Hanlon F M, Platten A, Farid M, Auerbach P, Braff D L, & Geyer M A. (2000). Toward understanding the biology of a complex phenotype: Rat strain and substrain differences in the sensorimotor gating-disruptive effects of dopamine agonists, The Journal of Neuroscience, 20(11), 4325-4336.
Swerdlow N R, Shoemaker J M, Platten A, Pitcher L, Goins J, Auerbach P P (2004). Heritable differences in the dopaminergic regulation of sensorimotor gating
I. Apomorphine effects on startle gating in albino and hooded outbred rat strains and their F1 and N2 progeny. Psychopharmacology, 174(4):441-451.
Swerdlow, N. R., Geyer, M. A., Braff, D. L. Neural circuit regulation of prepulse inhibition of startle in the rat: current knowledge and future challenges, Psychopharmacology 156 (2001) 194-215.
Takao T, Flint N, Lee L, Ying X, Merrill J, Chandross K J. 2004. 17beta-estradiol protects oligodendrocytes from cytotoxicity induced cell death. J Neurochem 89:660-673.
Tang M-T, Jacobs D, Stern Y, Marder K, Schofield P, Gurland B et al.: Effect of oestrogen during menopause on risk and age at onset of Alzheimer's disease. Lancet 1996, 348: 429-432.
Tate C G, Blakely R D. (1994) The effect of N-linked glycosylation on activity of the Na+ and Cl-dependent serotonin transporter expressed using recombinant baculovirus in insect cells. J. Biol. Chem. 269:26303-26310.
Thompson T L, Bridges S R, Weirs W J. (2001) Alteration of dopamine transport in the striatum and nucleus accumbens of ovariectomized and estrogen-primed rats following N-(p-isothiocyanatophenethyl) spiperone (NIPS) treatment. Brain Res. Bull. 54:631-638.
Thompson T L, Moss R L. 1994. Estrogen regulation of dopamine release in the nucleus accumbens: genomic and nongenomic-mediated effects. J Neurochem 229:145-148.
Thompson T L. 1999. Attenuation of dopamine uptake in vivo following priming with estradiol benzoate. Brain Res 834:164-167.
Toader V, Xu X, Nicolescu A, Yu L, Bolton J L, Thatcher G R J. 2003. Nitrosation, nitration and autoxidation of the selective estrogen receptor modulator raloxifene by nitric oxide, peroxynitrite and reactive nitrogen/oxygen species. Chem Res Toxicol 16:1264-1276.
Toggas S M, Masliah E, Rockenstien E M, Rall G F, Abraham C R, Mucke L. 1994. Central nervous system damage produced by expression of HIV-1 coat protein gp120 in transgenic mice. Nature 367:188-193.
Torres G E, Carneiro A, Seamans K, Fiorentini C, Sweeney A, Yao W E, Caron M G. (2003) Oligomerization and trafficking of the human dopamine transporter: mutational analysis identifies critical domains important for the functional expression of the transporter. J. Biol. Chem. 278:2731-2739.
Tsang K-L, Ho S-L, Lo S-K: Estrogen improves motor disability in parkinsonian postmenopausal women with motor fluctuations. Neurology 2000, 54: 2292-2298.
Turchan J, Anderson C, Hauser K, Sun Q, Zhang J, Liu Y, Wise P M, Kruman I I, Mattson M P, Booze R M, Maragos W, Nath A (2001) protection by estrogen of synergistic neurotoxicity of HIV proteins, methamphetamine and cocaine. BMC Neurosci. 2, 3.
Turchan J, Anderson C, Hauser K F, Sun Q, Zhang J, Liu Y et al.: Estrogen protects against the synergistic toxicity by HIV proteins, methamphetamine and cocaine. BMC Neurosci 2001, 2: 3.
UNAIDS (2004) AIDS Epidemic Update:2004. Geneva, Switzerland:UNAIDS.
Valle L D, Croul S, Morgello S, Amini S, Rappaport J, Khalili K (2000) Detection of HIV-1 Tat and JCV capsid protein, VP 1, in AIDS brain with progressive multifocal leukoencephalopathy. J Neurovirol 6: 221-228.
Valle, L. D., Croul, S., Morgello, S., Amini, S., Rappaport, J., Khalili, K. (2000) Detection of HIV-1 Tat and JCV capsid protein, VP1, in AIDS brain with progressive multifocal leukoencephalopathy. J. Neurovirol. 6, 221-228.
van de Bovenkamp M, Nottet H S L M, Pereira C F (2002). Interaction of human immunodeficiency virus-1 proteins with neurons: possible role in the development of human immunodeficiency virus-1-associated dementia. European Journal of Clinical Investigation, 32:619-627.
van Haaren F, Meyer M E (1991) Sex differences in locomotor activity after acute and chronic cocaine administration. Pharmacol Biochem Behav 39(4):923-927.
Van Hardesveldt, C. and Joyce, J. N. (1986) Effects of estrogen on the basal ganglia. Pharmacol. Biochem. Behav. 10:1-14.
Vaughan R A, Brown V L, McCoy M T, Kuhar M J. (1996) Species- and brain region-specific dopamine transporters: immunological and glycosylation characteristics. J. Neurochem. 66:2146-2152.
Vezina P (2004) Sensitization of midbrain dopamine neuron reactivity and the self-administration of psychomotor stimulant drugs. Neurosci Biobehav Rev 27:827-839.
Vongher J, Frye C: Progesterone in conjunction with estradiol has neuroprotective effects in an animal model of neurodegeneration. Pharmacol Biochem Behav 1999, 64: 777-785.
Wagner, G. C., Tekirian, T. L. and Cheo, C. T. (1993) Sexual differences in sensitivity to methamphetamine toxicity. J. Neural Transm. Gen. Sect. 93:67-70.
Wallace D R, Dodson S, Nath A, Booze R M (2006) Estrogen attenuation of gp120- and tat-induced oxidative stress: role of the dopamine transporter. Synapse 59(1):51-60.
Wallace D R, Dodson S, Nath A, Booze R M. (2005) Estrogen attenuates gp120- and tat(1-72)-induced oxidative stress and prevents loss of dopamine transporter function. Synapse; 59:51-60.
Wallace D R, Mactutus C F, Booze R M (1996) Repeated intravenous cocaine administration: locomotor activity and dopamine D2/D3 receptors. Synapse 23(3):152-163.
Wallace, D. R. (2005) Overview of molecular, cellular and genetic neurotoxicology. Neurol. Clin., in press. Review
Wallace, D. R. and Booze, R. M. (1995a) Identification of D3 and sigma receptors in the rat striatum and nucleus accumbens using [3H]-(+)-7-OH-DPAT and carbetapentane. J. Neurochem., 64:700-710.
Wallace, D. R. and Booze, R. M. (1995b) Pramipexole: A dopamine D3-preferring compound that lacks interactions with sigma binding sites. Neurosci. Res. Comm. 17:177-183.
Wallace, D. R., Dodson, S., Nath, A., Booze, R. M. (2004, submitted, in Appendix) Estrogen attenuation of gp120- and tat-induced oxidative stress: role of the dopamine transporter.
Wallace, D. R., Mactutus, C. F. and Booze, R. M. (1996) Repeated intravenous cocaine administration: Locomotor activity and dopamine D2/D3 receptors. Synapse, 23:152-163.
Wang G J, Chang L, Volkow N D, Telang F, Logan J, Ernst T, Fowler JS (2004) Decreased brain dopaminergic transporters in HIV-associated dementia patients. Brain 127(Pt 11):2452-2458.
Wang G J, Chang L, Volkow N D, Telang F, Logan J, Ernst T, Fowler JS. 2004. Decreased brain dopaminergic transporters in HIV-associated dementia patients. Brain 127:2452-2458.
Wang H, Joseph J A. 1999. Quantifying cellular oxidative stress by dichlorofluorescein assay using microplate reader. Free Rad Biol Med 27:612-616.
Wang J-G, Chang L, Volkow N, Telang F, Logan J, Ernst T et al. (2004) Decreased brain dopaminergic transporters in HIV-associated dementia patients. Brain; 127: 2452-2458.
Wang, G. J., Chang, L., Volkow, N. D., Telang, F., Logan, J., Ernst, T., Fowler, J. S. (2004) Decreased brain dopaminergic transporters in HIV-associated dementia patients. Brain. 127(Pt 11):2452-8.
Webber M, Shoenbaum E, Gourevitch M, Buono D, Klein R: A prospective study of HIV disease progression in female and male drug users. AIDS 1999, 13: 257-262.
Weill-Engerer S, David J-P, Sazdovitch V, Liere P, Schumacher M, Delacourte A et al.: In vitro metabolism of dehydroepiandrosterone (DHEA) to 7a-hydroxy-DHEA and D5-androstene-3b,17b-diol in specific regions of the aging brain from Alzheimer's and non-demented patients. Brain Res 2003, 969: 117-125.
Wersinger C, Banta M, Sidhu A. (2004) Comparative analyses of □-synuclein expression levels in rat brain tissues and transfected cells. Neurosci. Lett. 358:95-98.
Wersinger C, Prou D, Vernier P, Sidhu S. (2003) Modulation of dopamine transporter function by α-synuclein is altered by impairment of cell adhesion and by induction of oxidative stress. FASEB J. 17:2151-2153.
Wersinger C, Sidhu S. (2003) Attenuation of dopamine transporter activity by □-synuclein. Neurosci. Lett. 340:189-192.
Wersinger C, Vernier P, Sidhu A. (2004) Trypsin disrupts the trafficking of the human dopamine transporter by □-synuclein and its A30P mutant. Biochem. 43:1242-1253.
Wesselingh S L, Power C, Glass J D, Tyor W R, McArthur J C, Farber J M, Griffin J W, Griffin D E (1993) Intracerebral cytokine messenger RNA expression in acquired immunodeficiency syndrome dementia. Ann Neurol 33(6):576-582.
Wesselingh, S. L., Power, C., Glass, J. D., Tyor, W. R., McArthur, J. C., Farber, J. M., Griffin, J. W., Griffin, D. E. (1993) Intracerebral cytokine messenger RNA expression in acquired immunodeficiency syndrome dementia. Ann Neurol. 33(6):576-82.
Westendorp M O, Frank R, Ochsenbauer C, Stricker K, Dhein J, Walczak H, Debatin K M, Krammer P H (1995) Sensitization of T cells to CD95-mediated apoptosis by HIV-1 Tat and gp120. Nature 375:497-500.
Westendorp, M. O., Frank, R., Ochsenbauer, C., Stricker, K., Dhein, J., Walczak, H., Debatin, K. M., and Krammer, P. H. (1995). Sensitization of T cells to CD95-mediated apoptosis by HIV-1 Tat and gp120. Nature 375:497-500.
Wiley C A, Baldwin M, Achim C L (1996) Expression of HIV regulatory and structural mRNA in the central nervous system. AIDS 10(8):843-847.
Wiley, C. A., Baldwin, M., Achim, C. L. (1996) Expression of HIV regulatory and structural mRNA in the central nervous system. AIDS. 10(8):843-7.
Winer B J (1971) Statistical Principles in Experimental Design. New York: McGraw-Hill.
Winer, B. J., (1971) Statistical Principles in Experimental Design, 2nd ed., McGraw-Hill, NY.
Wise R A, Bozarth M A (1985) Brain mechanisms of drug reward and euphoria. Psychiatr. Med 3:445-460.
Woolley C: Effects of estrogen in the CNS. Curr Opin Neurbiol 1999, 9: 349-354.
Wooten G, Curie L, Bovbj erg V, Lee J, Patrie J: Are men at greater risk for Parkinson's disease than women? J Neurosurg Psychiatry 2004, 75: 637-639.
Wright, S. N., Wang, S. Y., Wang, G. K. (1998) Lysine point mutations in Na+ channel D4-S6 reduce inactivated channel block by local anesthetics. Mol. Pharmacol. 54, 733-739.
Xia S, Cai Z Y, Thio L L, Kim-Han J S, Dugan L L, Covey D F, Rothman S M. 2002. The estrogen receptor is not essential for all estrogen neuroprotection: new evidence from a new analog. Neurobiol Dis 9:282-293.
Yang S-H, Liu R, Wu S, Simpkins J: The use of estrogens and related compounds in the treatment of damage from cerebral ischemia. Ann N Y Acad Sci 2003, 1007: 101-107.
Yeung M C, Geertsma F, Liu J, Lau A S. 1998. Inhibition of HIV-1 gp120-induced apoptosis in neuroblastoma SK-N-SH cells by an antisense oligodeoxynucleotide against p53. AIDS 12:349-354.
Yu, Y. L. and Wagner, G. C. (1994) Influence of gonadal hormones on sexual differences in sensitivity to methamphetamine-induced neurotoxicity. J. Neural. Transm. 8:215-221.
Zahniser N R, Peris J (1992) Neurochemical mechanisms of cocaine-induced sensitization. In: Cocaine: Pharmacology, physiology, and clinical strategies (Lakosli J M, Galloway M P, White F J, eds), pp 229-260 Boca Raton, Fla.: CRC Press.
Zang, Z., Rydel, R. E., Drzewiecki, G. J., Fuson, K., Wright, S., Wogulius, M., Audia, J. E., May, P. C., Hyskop, P. A. (1996) Amyloid □-mediated oxidative and metabolic stress in rat cortical neurons: no direct evidence for a role for H2O2 generation. J. Neurochem. 67, 1595-1606.
Zauli G, Secchierro P, Rodella L, Gibellini D, Mirandola P, Mazzoni M et al. (2000) HIV-1 Tat-mediated inhibition of the tyrosine hydroxylase gene expression in dopaminergic neuronal cells. Journal of Biological Chemistry; 275: 4159-4165.
Zaulyanov L L, Green P S, Simpkins J W. 1999. Glutamate receptor requirements for neuronal death from anoxia-reoxygenation: an in vitro model for assessment of the neuroprotective effects of estrogen. Cell Mol Neurobiol 19:705-718.
Zemlyak I, Brooke S M, Sapolsky R M. 2002. Protection against gp120-induced neurotoxicity by an array of estrogenic steroids. Brain Res 958:272-276.
Zwain I H, Yen SSC, Cheng C Y: Astrocytes cultured in vitro produce estradiol-17b and express aromatase cytochrome P-450 (P-450 AROM) mRNA. Biochim Biophys Acta 1997, 1334: 338-348.
Zwain I H, Yen S S C: Dehydroepiandrosterone: Biosynthesis and metabolism in the brain. Endocrinology 1999, 140: 880-887.

1. A method of protecting a neuron from dysfunction induced by an HIV neurotoxin, comprising contacting the cell with a therapeutically effective dose of a dopamine D1 receptor agonist.

2. A method of treating or preventing HIV-1 associated dementia (HAD) in a subject in need of such treatment or prevention, comprising administering to the subject a therapeutically effective dose of a dopamine D1 receptor agonist.

3. The method of claim 1, wherein the dopamine D1 receptor agonist is not dopamine.

4. The method of claim 1, wherein the dopamine D1 receptor agonist does not increase HIV-1 replication.

5. The method of claim 1, wherein the dopamine D1 receptor agonist does not bind the dopamine D2 receptor.

6. The method of claim 1, wherein the dopamine D1 receptor agonist does not bind the dopamine D3 receptor.

7. The method of claim 1, wherein the dopamine D1 receptor agonist does not bind the dopamine D4 receptor.

8. The method of claim 1, wherein the dopamine D1 receptor agonist does not bind the dopamine D5 receptor.

9. The method of claim 1, wherein the dopamine D1 receptor agonist is selected from the group consisting of Bromocriptine, Pergolide, Ropinirole, Pramipexole, Entacapone, Tolcapone, Fenoldopam, Apomorphine, Dihexadine, IPX-750, Cabergoline, A68930, SKF38393, CY208-243, SKF81297, NNC01-0012, and SCH23390.

10. The method of claim 2, wherein the HAD in the subject is a result of HIV-1 infection in the presence of a psychostimulant.

11. The method of claim 10, wherein the psychostimulant is cocaine or methamphetamine.

12. The method of claim 1, further comprising contacting the cell with a therapeutic amount of an estrogen receptor agonist.

13. The method of claim 12, wherein the estrogen receptor agonist is selected from the group consisting of 17 beta-estradiol, conjugated equine estrogens, synthetic conjugated estrogens, esterified estrogens, and testosterone.

14. The method of claim 13, wherein the estrogen receptor agonist is administered in combination with Raloxifene or a Progestogen.

15. The method of claim 1, further comprising contacting the cell with a therapeutic amount of a compound that decreases dopamine availability.

16. The method of claim 1, further comprising contacting the cell with an antiretroviral compound.

17. The method of claim 16, wherein the antiretroviral compound comprises one or more molecules selected from the group consisting of protease inhibitors [PI], nucleoside reverse transcriptase inhibitors [NRTI], and non-nucleoside reverse transcriptase inhibitors [NNRTI].

18. The method of claim 17, wherein the PI is selected from the group consisting of Indinavir, Amprenavir, Nelfinavir, Saquinavir, Fosamprenavir, Lopinavir, Ritonavir, and Atazanavir.

19. The method of claim 17, wherein the NRTI is selected from the group consisting of Abacavir, Stavudine, Didanosine, Lamivudine, Zidovudine, Zalcitabine, Tenofovir, and Emtricitabine.

20. The method of claim 17, wherein the NNRTI is selected from the group consisting of Efavirenz, Nevirapine, and Delavirdine.

21. The method of claim 2, wherein the dopamine D1 receptor agonist is not dopamine.

22. The method of claim 2, wherein the dopamine D1 receptor agonist does not increase HIV-1 replication.

23. The method of claim 2, wherein the dopamine D1 receptor agonist does not bind the dopamine D2 receptor.

24. The method of claim 2, wherein the dopamine D1 receptor agonist does not bind the dopamine D3 receptor.

25. The method of claim 2, wherein the dopamine D1 receptor agonist does not bind the dopamine D4 receptor.

26. The method of claim 2, wherein the dopamine D1 receptor agonist does not bind the dopamine D5 receptor.

27. The method of claim 2, wherein the dopamine D1 receptor agonist is selected from the group consisting of Bromocriptine, Pergolide, Ropinirole, Pramipexole, Entacapone, Tolcapone, Fenoldopam, Apomorphine, Dihexadine, IPX-750, Cabergoline, A68930, SKF38393, CY208-243, SKF81297, NNC01-0012, and SCH23390.

28. The method of claim 2, further comprising administering to the subject a therapeutic amount of an estrogen receptor agonist.

29. The method of claim 28, wherein the estrogen receptor agonist is selected from the group consisting of 17 beta-estradiol, conjugated equine estrogens, synthetic conjugated estrogens, esterified estrogens, and testosterone.

301. The method of claim 29, wherein the estrogen receptor agonist is administered in combination with Raloxifene or a Progestogen.

31. The method of claim 2, further comprising administering to the subject a therapeutic amount of a compound that decreases dopamine availability.

32. The method of claim 2, further comprising administering to the subject an antiretroviral compound.

33. The method of claim 32, wherein the antiretroviral compound comprises one or more molecules selected from the group consisting of protease inhibitors [PI], nucleoside reverse transcriptase inhibitors [NRTI], and non-nucleoside reverse transcriptase inhibitors [NNRTI].

34. The method of claim 33, wherein the PI is selected from the group consisting of Indinavir, Amprenavir, Nelfinavir, Saquinavir, Fosamprenavir, Lopinavir, Ritonavir, and Atazanavir.

35. The method of claim 33, wherein the NRTI is selected from the group consisting of Abacavir, Stavudine, Didanosine, Lamivudine, Zidovudine, Zalcitabine, Tenofovir, and Emtricitabine.

36. The method of claim 33, wherein the NNRTI is selected from the group consisting of Efavirenz, Nevirapine, and Delavirdine.
